(19) United States
(12) Patent Application Publication
(10) Pub. No.: US 2025/0082237 A1
(43) Pub. Date: Mar. 13, 2025

(54) SYSTEMS AND METHODS FOR SAMPLE COLLECTION

(71) Applicant: Drawbridge Health, Inc., Summerville, SC (US)

(72) Inventor: Jerome Scelza, New York, NY (US)

(21) Appl. No.: 18/612,795

(22) Filed: Mar. 21, 2024

Related U.S. Application Data
(63) Continuation of application No. PCT/US2022/044612, filed on Sep. 23, 2022.
(60) Provisional application No. 63/247,721, filed on Sep. 23, 2021.

(51) Int. Cl. A61B 5/15

(52) U.S. Cl. CPC A61B 5/150343; A61B 5/150206; A61B 5/150755

(57) ABSTRACT

Disclosed herein are systems, methods, and kits for collecting and storing a sample from a subject. A system can comprise a cartridge assembly for separation of the blood. The cartridge assembly can comprise a cartridge port configured to couple to a sample acquisition device that is usable to collect the blood from the subject. The cartridge assembly can comprise at least one blood separation membrane that is configured to separate plasma or serum from the blood. In some cases, the cartridge port can comprise a pathway that is configured to direct the blood to flow from the sample acquisition device, through the pathway, and towards the cartridge assembly. In some cases, a direction of flow of the blood through the at least one blood separation membrane can be different from a direction of flow of the blood through the pathway and towards the inner portion of the cartridge assembly.

### CROSS-REFERENCE

This application is a continuation application of International Patent Application No. PCT/US2022/044612, filed Sep. 23, 2022, which claims the benefit of U.S. Provisional Patent Application No. 63/247,721, filed Sep. 23, 2021, each of which is entirely incorporated herein by reference.

### BACKGROUND

Body fluid collection, for example collection of blood samples for performing diagnostic tests, can be used to assess and inform the health of individuals. Early detection and reliable diagnosis can play a central role in making effective therapeutic decisions for treatment of diseases or managing certain physiological conditions. Detection can involve identification of disease-specific biomarkers in human body fluids that can indicate irregularities in cellular regulatory functions, pathological responses, or intervention to therapeutic drugs.

Many individuals, however, may not relish the process of having blood drawn from their bodies, possibly due to association with pain, cuts, bleeding, sharp objects, sight of blood, fear of infections, etc. Typically, venous blood collection of a subject is performed at external facilities such as hospitals, skilled nursing facilities, and outpatient environments such as primary care physician (PCP) & specialty hospital clinics, surgery centers, occupational health clinics, or physician offices. The blood collection process can be tedious and time consuming for individuals who have to visit those facilities for blood draw, and for healthcare personnel who can have to attend to multiple patient encounters within a single day.

Thus, a need exists for improved devices and methods that enable blood collection to be performed easily and conveniently by users and that can decrease users' reliance on traditional healthcare facilities for blood draw.

### SUMMARY

The present disclosure addresses at least the above needs. Various embodiments of the present disclosure address the demand for devices and methods, that enable individuals to easily, conveniently, and reliably collect and store blood samples outside of traditional healthcare facilities, for example in their own homes, in remote locations, while traveling, etc. Individuals who have minimal to no medical training can use the disclosed devices and methods to efficiently collect and store blood on their own or with the help of others, without the need for trained healthcare personnel. The embodiments described herein can obviate the need for individuals to schedule or make special or frequent trips to healthcare facilities for blood sample collection, which helps to free up the individuals' time and reduce patient load on healthcare resources. Nonetheless, it should be appreciated that the disclosed devices and methods are also suitable for use by healthcare or non-healthcare personnel in a variety of environments or applications, for example in personalized point-of-care (POC), Emergency Medical Services (EMS), ambulatory care, hospitals, clinics, emergency rooms, patient examination rooms, acute care patient rooms, field environments, nurse's offices in educational settings, occupational health clinics, surgery or operation rooms, etc.

Blood samples collected using the devices and methods described herein can be analyzed to determine a person's physiological state, for detecting diseases and also for monitoring the health conditions of an individual or a subject. In some instances, individuals can rapidly evaluate their physiological status since blood samples can be quickly collected using the devices and methods described herein, and either (1) analyzed on the spot using for example immunoassays or (2) shipped promptly to a testing facility. The reduced lead-time for blood collection, analysis and quantification can be beneficial to many users, especially subjects who have certain physiological conditions/diseases that require constant and frequent blood sample collection/monitoring. Taking diabetes as an example, hemoglobin A1c (HbA1c) can make up 60% of all glycohemoglobins and can be used for monitoring glycemic control. The amount of HbA1c, as a percentage of total hemoglobin, can reflect the average blood glucose concentration in a patient's blood over the preceding 120 days. Generally, it is recommended that diabetic patients test their HbA1c levels every three to six months. The glycemic recommendation for non-pregnant adults with diabetes can be <7.0%, while HbA1c levels of >8% can indicate that medical action can be required to control diabetic complications, including cognitive impairment and hypoglycemic vulnerability.

The various embodiments described herein are capable of drawing blood at increased flowrates and higher sample volumes beginning from time of skin incision, compared to traditional non-venous blood collection devices and method. The disclosed devices and methods can be used to collect blood samples of predefined volumes, for example through the use of custom matrices for sample collection, and absorbent pads for holding and metering out excess blood. Additionally, the blood collection devices and methods described herein are minimally invasive and permit lower levels of pain (or perception of pain) in a subject, which can help to improve the overall blood collection experience for the subject.

An aspect of the present disclosure provides a cartridge assembly for separation of blood collected from a subject, the cartridge assembly comprising: a cartridge port configured to couple to a sample acquisition device that is usable to collect the blood from the subject; at least one blood separation membrane configured to separate plasma or serum from the sample; and a slot configured to support the at least one blood separation membrane, wherein the cartridge port comprises a pathway that is configured to direct the blood to flow from the sample acquisition device into a proximal end of the pathway in a first direction, through the pathway, and exit from a distal end of the pathway onto the at least one blood separation membrane in a second direction that is different from the first direction.

The pathway can comprise a groove or a channel. An angle between the first direction and a longitudinal axis of the cartridge assembly can be greater than zero degree and less than 180 degrees. An angle between the second direction and a longitudinal axis of the cartridge assembly can be greater than zero degree and less than 180 degrees. An angle of intersection between the first direction and the second direction can be greater than zero degree and less than 180 degrees.

The slot can be further configured to support a collection media for collecting the separated plasma or serum. The slot can be further configured to support a pre-filter for filtering the blood prior to separating the plasma or serum from the blood. The at least one blood separation membrane, the collection media, and the pre-filter can be provided as a stack within the slot. The stack can be disposed in a configuration that permits lateral flow of the blood through a thickness of the stack in a third direction, and across a planar area of the stack in at least one other direction that is different from the third direction. The third direction can be different from the first direction or the second direction. The third direction can be substantially orthogonal to a longitudinal axis of the cartridge. The third direction and the at least one other direction can be substantially orthogonal to one another. The distal end of the pathway can be configured to direct the blood to a planar surface of the pre-filter before the blood flows onto the at least one blood separation membrane.

The proximal end of the pathway can be configured to receive the blood from a recessed opening in the housing of the sample acquisition device. The proximal end and the distal end of the pathway may not lie along a longitudinal axis of the cartridge assembly. The proximal end and the distal end of the pathway may not lie along a straight line extending between the proximal end and the distal end. The distal end of the pathway can be offset from a linear axis extending between (1) the proximal end of the pathway and (2) an edge thickness portion of the stack located between the proximal end and the distal end of the pathway. The distal end of the pathway can be adjacent to but not in contact with the planar surface of the pre-filter.

The pathway can comprise a bent, curved, or angled portion.

The pathway can comprise a cut-out exposing a portion along a length of the inlet port. The cartridge can be subject to vacuum pressure when a vacuum in the sample acquisition device is activated. The vacuum can be configured to assist with the lateral flow of the blood through and/or across the stack.

The slot can further comprise an accumulation region, wherein the accumulation region can be configured to hold a volume of the blood to contain the blood as it is being absorbed into at least a portion of the at least one blood separation membrane. The accumulation region can be disposed adjacent to the pre-filter. The accumulation region can be configured to hold a predetermined volume of the blood.

The cartridge can be configured to be released and decoupled from the sample acquisition device after the plasma or serum has been separated and collected onto the collection media. The collection media can be configured to be released and decoupled from the cartridge assembly after the plasma or serum has been separated and collected onto the collection media. The cartridge assembly can be configured to remain coupled to the sample acquisition device after the collection media has been released and decoupled from the cartridge assembly.

The at least one blood separation membrane can comprise a plurality of blood separation membranes, and the collection media can be disposed between the plurality of blood separation membranes.

The cartridge assembly can further comprise a window that permits a user to observe a progress of the blood separation. The window can be located adjacent to the at least one blood separation membrane, the collection media, or the pre-filter.

The at least one blood separation membrane can comprise an anti-coagulant. The cartridge assembly can further comprise an anticoagulant coupled to a surface of the pathway.

Another aspect of the present disclosure provides a cartridge assembly for separation of blood collected from a subject, the cartridge assembly comprising: a cartridge port configured to couple to a sample acquisition device that is usable to collect the blood from the subject; at least one blood separation membrane configured to separate plasma or serum from the blood; and a slot configured to support the at least one blood separation membrane, wherein the cartridge port comprises a pathway that is configured to direct the blood to flow from the sample acquisition device, through the pathway, and towards an inner portion of the cartridge assembly comprising the slot, and wherein (i) a direction of flow of the blood through the at least one blood separation membrane is different from (ii) a direction of flow of the blood through the pathway and towards the inner portion of the cartridge assembly.

The direction of flow of the blood through the at least one blood separation membrane can be substantially orthogonal to the direction of flow of the blood through the pathway.

The slot can be further configured to support one or both of (1) a collection media for collecting the separated plasma or serum and (2) a pre-filter for filtering the blood prior to separating the plasma or serum from the blood. The at least one blood separation membrane can be disposed between the collection media and the pre-filter.

Another aspect of the present disclosure provides a cartridge assembly for separation of blood collected from a subject, the cartridge assembly comprising: a cartridge port configured to couple to a sample acquisition device that is usable to collect the blood from the subject; at least one blood separation membrane configured to separate plasma or serum from the blood; a slot configured to support the at least one blood separation membrane; and a collection reservoir configured to contain the blood collected from the sample acquisition device prior to or during the plasma or serum separation by the at least one blood separation membrane, wherein the cartridge port comprises a pathway that is configured to direct the blood to flow from the sample acquisition device, through the pathway, and towards the collection reservoir.

A direction of flow of the blood through the at least one blood separation membrane can be different from a direction of flow of the blood through the pathway and towards the collection reservoir. The direction of flow of the blood through the at least one blood separation membrane can be substantially orthogonal to the direction of flow of the blood through the pathway and towards the collection reservoir. The collection reservoir can be disposed adjacent to a planar surface the at least one blood separation membrane.

The slot can be further configured to support one or both of (1) a collection media for collecting the separated plasma or serum and (2) a pre-filter for filtering the blood prior to separating the plasma or serum from the blood. The at least one blood separation membrane can be disposed between the collection media and the pre-filter. The collection reservoir can be disposed adjacent to a planar surface of the pre-filter.

Another aspect of the present disclosure provides a system for blood collection and blood separation, comprising: any of the subject sample acquisition device and cartridge assembly of the present disclosure.

The sample acquisition device can comprise an onboard vacuum.

Another aspect of the present disclosure provides a method comprising: using any of the subject sample acquisition device of the present disclosure to collect the blood from the subject; and using any of the subject cartridge assembly of the present disclosure to separate the plasma or serum from the blood.

Another aspect of the present disclosure provides a cartridge assembly for storing liquid blood collected from a subject, the cartridge assembly comprising: a coupling unit configured to couple to a cartridge chamber of a sample acquisition device, wherein the sample acquisition device is configured to collect the blood from the subject; a container configured to store the liquid blood; and a cartridge holder configured to support the container, where a proximal end of the container is configured to couple to the coupling unit, and a distal end of the container is configured to couple to the cartridge holder.

The container can comprise a cap coupled to the proximal end of the container, and the proximal end of the container can be configured to couple to the coupling unit using the cap. The cap can comprise one or more openings that are configured to open and permit fluidic access to the container when the cap is coupled to the coupling unit. The one or more openings can be further configured to close and prohibit the fluidic access to the container when the cap is decoupled from the coupling unit. The coupling unit can comprise one or more fluidic pathways that permit air to expunge out of the container and into the cartridge chamber as the blood is being collected into the container. The one or more fluidic pathways can comprise one or more venting grooves or channels. The one or more fluidic pathways can be configured to allow vacuum pressure within the cartridge chamber to be equalized as the blood is being collected into the container. The container can be configured to receive the blood flowing into the container in a first direction, and the one or more fluidic pathways can be configured to direct and expunge the air out of the container in a second direction that is different from the first direction. The first direction and the second direction can be substantially opposite to each other. The first direction and the second direction can be substantially orthogonal to each other.

A portion of the cartridge holder can be configured to extend outside of the cartridge chamber when the cartridge assembly is coupled to the cartridge chamber. The portion of the holder can comprise a cartridge tab.

The cartridge holder can comprise a gasket that is configured to hermetically seal the cartridge chamber when the cartridge assembly is coupled to the cartridge chamber.

The container and the cartridge holder can comprise a set of interlocking mating features that permits the container to be secured to the holder.

The cartridge chamber can be under vacuum pressure as a result of activating a vacuum in the sample acquisition device. The vacuum can be configured to assist with flow of the blood from a recessed opening in the housing of the sample acquisition device into the container.

At least a portion of the cartridge assembly can be configured to be released and decoupled from the cartridge chamber of the sample acquisition device after the blood has been collected into the container.

The container can be configured to be released and decoupled from the coupling unit after the blood has been collected into the container.

The container can comprise a window that permits a user to observe a progress of the liquid blood collection.

The cartridge assembly can further comprise: one or more sensors that are configured to detect an amount of the blood collected in the container. The one or more sensors can comprise an optical sensor. The one or more sensors can be in communication with an electronic fill indicator, and the electronic fill indicator is configured to provide information to a user about the amount of the blood that is collected in the container. The electronic fill indicator can be configured to generate one or more visual, audible, or tactile signals. The electronic fill indicator can be located on or with the cartridge. The electronic fill indicator can be located on or with the sample acquisition device.

The coupling unit can comprise a luer-style fitting.

The container can comprise one or more indicator lines that are used for monitoring a progress of the liquid blood collection.

The one or more indicator lines can be used for estimating an amount of the blood that is collected in the container.

The container can comprise a tube.

Another aspect of the present disclosure provides a system for collecting and storing blood from a subject, comprising: any of the subject sample acquisition device and the cartridge assembly of the present disclosure. The sample acquisition device comprises an onboard vacuum.

Another aspect of the present disclosure provides a method comprising: using any of the subject sample acquisition device of the present disclosure to collect the blood from the subject; and using any of the subject cartridge assembly of the present disclosure to store the blood as liquid blood.

Another aspect of the present disclosure provides a modular chamber assembly for storing blood collected from a subject, the modular assembly comprising: an inlet port configured to couple to a sample acquisition device, wherein the sample acquisition device is configured to collect the blood from the subject; and a chamber configured to couple to the inlet port, wherein an enclosure is formed when the chamber is coupled to the inlet port, wherein the enclosure is configured to support therein a cartridge assembly of a plurality of different cartridge assembly types, and wherein the plurality of different cartridge assembly types permits the blood to be collected, processed or stored in a plurality of different formats comprising plasma, serum, dried blood, liquid blood, or coagulated blood.

A portion of the sample acquisition device can be configured to extend out of the sample acquisition device when the inlet port is coupled to a mating port of the sample acquisition device. The portion of the sample acquisition device can comprise a protrusion.

The inlet port can comprise a pierceable self-sealing port that is configured to hermetically seal the enclosure.

The cartridge assembly can be configured to couple to (1) at least a portion of the inlet port and/or (2) at least a portion of the chamber.

The plurality of different cartridge assembly types can comprise two or more of the following: (1) a first cartridge assembly type configured to separate the plasma or serum from the collected blood, (2) a second cartridge assembly type configured to collect and store the liquid blood, (3) a third cartridge assembly type configured to hold one or more matrices for collecting and storing the blood as the dried blood, or (4) a fourth cartridge assembly type configured to store coagulated blood.

The first cartridge assembly type can be configured to separate the plasma from the collected blood. The first cartridge assembly type can be configured to separate the serum from the collected blood.

The modular chamber assembly can be configured to be released and detached from the sample acquisition device when the inlet port is decoupled from the sample acquisition device.

The modular chamber assembly can be configured to be released and detached from the sample acquisition device after the blood is collected, processed or stored on the cartridge assembly.

The chamber can be configured to protect the cartridge assembly from an external environment after the blood is collected, processed or stored on the cartridge assembly and after the modular chamber assembly is released and detached from the sample acquisition device.

The chamber can be in a shape of a tube.

The modular chamber assembly can be configured to be used as a transport container for shipping or transporting the blood after the blood is collected, processed or stored on the cartridge assembly.

The chamber can comprise a desiccant.

The chamber can comprise a transparent or semi-transparent window to allow visualization of an inner portion of the chamber.

Another aspect of the present disclosure provides a system for collecting and storing blood from a subject, comprising: any of the subject sample acquisition device and modular chamber assembly of the present disclosure.

The sample acquisition device can comprise an onboard vacuum.

The modular chamber assembly can comprise an onboard vacuum.

A complete coupling of the sample acquisition device and the modular chamber assembly can be configured to activate sufficient vacuum for the collecting and storing of the blood from the subject.

Another aspect of the present disclosure provides a method comprising: using any of the subject sample acquisition device of the present disclosure to collect the blood from the subject; and using any of the subject modular chamber assembly of the present disclosure to store the blood in one of the plurality of different formats.

Another aspect of the present disclosure provides a kit comprising: any of the subject sample acquisition device, modular chamber assembly, and/or plurality of different cartridge assembly types of the present disclosure.

Another aspect of the present disclosure provides a sample acquisition device for collecting blood from a subject, the sample acquisition device comprising: a body comprising a recess having an opening; one or more piercing elements that are extendable through the opening to penetrate skin of the subject to enable collection of the blood into the sample acquisition device while the skin is drawn into the recess; and a sample chamber comprising a connection port, wherein the connection port is sized and shaped to interchangeably and releasably couple to a cartridge assembly of a plurality of different cartridge assembly types, wherein the plurality of different cartridge assembly types permits the blood to be collected, processed or stored in a plurality of different formats comprising dried plasma, liquid plasma, dried serum, liquid serum, dried blood, liquid blood, or coagulated blood.

The body can be operatively coupled to a vacuum chamber. The vacuum chamber may be configured such that activation of the vacuum causes fluidic communication to be established between the vacuum chamber and the recess to draw the skin of the subject into the recess, and the recess may serve as a suction cavity for drawing the skin.

The modular chamber assembly can comprise an onboard vacuum. The modular chamber assembly may be configured such that coupling of the modular chamber assembly to the body may cause fluidic communication to be established between the modular chamber assembly and the recess to draw the skin of the subject into the recess, and the recess may serve as a suction cavity for drawing the skin.

The cartridge assembly can be configured to releasably couple to the body.

The sample chamber can be hermetically sealed when the cartridge assembly is coupled to the connection port of the sample chamber.

The plurality of different cartridge assembly types can comprise two or more of the following: (1) a first cartridge assembly type configured to separate the plasma or serum from the collected blood, (2) a second cartridge assembly type configured to collect and store the liquid blood, (3) a third cartridge assembly type configured to hold one or more matrices for collecting and storing the blood as the dried blood, or (4) a fourth cartridge assembly type configured to store coagulated blood.

Another aspect of the present disclosure provides a kit comprising: a sample acquisition device configured to collect blood from a subject, wherein the sample acquisition device comprises a port that is sized and shaped to interchangeably and releasably couple to a cartridge assembly of a plurality of different cartridge assembly types; and the plurality of different cartridge assembly types, wherein the plurality of different cartridge assembly types comprise two or more of the following: (1) a first cartridge assembly type configured to separate plasma or serum from the collected blood, (2) a second cartridge assembly type configured to store the blood in a liquid form, (3) a third cartridge assembly type configured to hold one or more matrices for storing the blood in a substantially dried state, or (4) a fourth cartridge assembly type configured to store coagulated blood.

The kit can further comprise a sample chamber, wherein the cartridge assembly of the plurality of different cartridge assembly types is contained within the sample chamber, and the sample chamber is sized and shaped to interchangeably and releasably couple to the cartridge assembly. The sample chamber can comprise an onboard vacuum.

The sample acquisition device can comprise an onboard vacuum.

Provided herein are medical systems, devices, and methods for sample collection and storage. The disclosed systems, devices, and methods comprise structural features that facilitate sample collection (e.g. blood collection devices) as well as components for collecting blood samples onto a substrate (e.g. a matrix) for storage and transport.

Any of the devices disclosed herein can utilize generation of a vacuum to apply negative pressure to deform the skin of a subject and to apply local suction directly to the sample collection site, thereby facilitating sample flow and collection. Any of the devices disclosed herein can comprise a recess (e.g., a concave cavity) that can be placed at the surface of the skin of the subject. The recess can be configured to deliver vacuum (e.g., negative pressure, suction, etc.) to the skin of the subject. Any of the devices disclosed herein can comprise an opening disposed at the apex of, or other surface of the recess. The opening can be customized to allow a piercing element to pierce the skin of the subject. The piercing element can be configured to pass through an inner diameter of the opening. Local suction can be applied to the sample collection site through the opening and using the recess.

A vacuum can be configured to deform the skin of the subject using different mechanisms, for example the vacuum can be configured to draw the skin of the subject into the recess (e.g., a concave cavity). The concave cavity can be configured to constrain the surface of the skin against its entire concave surface (or a portion of its concave surface), at which point the piercing element can be used to pierce the skin of the subject. An opening contiguous with a fluidic pathway (e.g., a flow channel in fluidic communication with a cartridge) can draw the blood from the subject into the device when the vacuum is applied to the skin of the subject, and after an incision has been made in the skin of the subject.

Vacuum pressure can be generated using an evacuated vacuum chamber configured such that activation of the evacuated vacuum chamber forms negative pressure that draws the blood from the subject through the opening and channels of the device, and into a sample chamber that collects the subject's sample. The sample chamber can collect the subject's liquid sample (e.g., liquid blood). The sample chamber can comprise one or more cartridges to collect other types or formats of the subject's sample (e.g., plasma or serum). In some cases, a cartridge can comprise a solid matrix for sample collection and/or storage. The vacuum pressure(s) can be below ambient pressure (i.e., under vacuum conditions), e.g., in the range of between 1-20 psi below ambient pressure. The vacuum pressure can be about 5 psi below ambient pressure. Vacuum chamber volume can be within a 10%-100% margin of twice the total volumes of a plurality of factors comprising two or more of: a concave cavity, opening, channel, and at least a portion of a sample chamber Any of the devices disclosed herein can comprise a vacuum activation actuator that can be configured to activate the vacuum upon actuation of the vacuum activation actuator. The vacuum activation actuator can comprise a button located on the device or on a cartridge chamber. Alternatively or in addition to the above embodiments, vacuum pressure can be generated by insertion of a sample chamber that comprises an evacuated vacuum chamber. Insertion (or coupling) of the sample chamber into a sample acquisition device can initiate vacuum venting from the vacuum chamber into the device, thereby forming negative pressure (e.g., below ambient pressure) within the device and at least at least a portion of the sample chamber. The negative pressure can be configured such that it is sufficient to draw the skin of the subject into the recess (e.g., concave cavity) of the device. The piercing element of the device can be activated to pierce the skin of the subject, and subsequently, the pressure differential can draw the blood from the subject through the device and into at least a portion of the sample chamber.

Another aspect of the present disclosure provides for a cartridge assembly for separation of blood collected from a subject, the cartridge assembly comprising: a cartridge comprising a cartridge port, wherein the cartridge is configured to couple via the cartridge port to a sample acquisition device that is usable to collect a blood sample from the subject; a cartridge tab comprising a substrate; and a treatment/stabilization unit supported between the cartridge and the substrate of the cartridge tab, wherein the treatment/stabilization unit comprises a multi-piece collection matrix that is configured to separate plasma or serum from the blood sample, wherein the multi-piece collection matrix comprises at least one sub-matrix that has a different size or shape than one or more other sub-matrices of the multi-piece collection matrix.

The multi-piece collection matrix may comprises at least three sub-matrices. The multi-piece collection matrix may further be configured to store the plasma or serum that is separated from the blood sample. The multi-piece collection matrix may further be configured to stabilize the plasma or serum that is separated from the blood sample. A portion of the at least one sub-matrix of the multi-piece collection matrix may be exposed to an ambient environment. The portion of the at least one sub-matrix of the multi-piece collection matrix is located at a portion of the treatment/stabilization unit that is distal to the cartridge port. The portion of the at least one sub-matrix of the multi-piece collection matrix may be in contact with the substrate. The portion of the at least one sub-matrix of the multi-piece collection matrix may not in contact with the cartridge. A surface area of the portion of the at least one sub-matrix of the multi-piece collection matrix may be from about 100 mm² to about 150 mm². The portion of the at least one sub-matrix may be detachable from the multi-piece collection matrix. The cartridge and the substrate of the cartridge tab may be configured to support the treatment/stabilization unit in a configuration that enables the cartridge assembly to be used or operated in a substantially vertical orientation. The cartridge assembly may be configured to be used or operated at an angle from about 40 degrees to about 140 degrees relative to a horizontal plane. The cartridge assembly may be configured to be used or operated at an angle from about 60 degrees to about 120 degrees relative to a horizontal plane. The cartridge further comprises a compression region that may be configured to apply a compression force to a portion of the multi-piece collection matrix. The compression force may be from about 1 pound to about 10 pounds. The compression force may be usable to improve or control the flow of the blood sample across or through the multi-piece collection matrix. The compression force may be configured to hold or maintain the portion of the multi-piece collection matrix at a compressed thickness that is about 30% to about 90% of an uncompressed thickness of the portion of the multi-piece collection matrix. The compression force is configured to hold or maintain the portion of the multi-piece collection matrix at a thickness of about 0.75 mm to about 1.0 mm. The cartridge may further comprise a compression stop that is configured to limit (a) the compression force to less than or equal to a predetermined value and/or (b) a compressed thickness of the portion of the multi-piece collection matrix to less than or equal to a predetermined thickness. The cartridge further comprises one or more vents that are configured to allow fluidic communication between the multi-piece collection matrix and an external ambient environment. The one or more vents are configured to control a plasma concentration during separation of the blood sample by the multi-piece collection matrix. The one or more vents are configured to control a rate of desiccation during separation of the blood sample by the multi-piece collection matrix. The portion of the at least one sub-matrix of the multi-piece collection matrix is not subject to a compression force. At least one other portion of the multi-piece collection matrix is subject to the compression force.

In another aspect, disclosed is an apparatus comprising: an elongated strip having a dimensional aspect ratio of at least about 1:3 to about 1:10, wherein the elongated strip comprises a plurality of integrated layers or membranes for facilitating collection and processing of a sample.

In some embodiments, the elongated strip comprises a first portion for collecting blood cells and a second portion for collecting plasma.

In some embodiments, the first portion is adjacent to the second portion.

In some embodiments, the first portion is located upstream of the second portion along a direction of flow of the sample.

In some embodiments, the sample comprises the blood cells and the plasma.

In some embodiments, the dimensional aspect ratio provides an elongated flow path for the sample, which flow path enables a separation of the sample into a first portion comprising blood and a second portion comprising plasma.

In some embodiments, the plurality of integrated layers or membranes form a monolithic membrane configured to separate blood cells from plasma and stabilize the blood cells and the plasma.

In some embodiments, the plurality of integrated layers or membranes are treated with one or more reagents to (i) aid in detection of plasma, (ii) enhance plasma separation across a plurality of regions according to a predetermined ratio, or (iii) stabilize a whole blood region or a plasma region of the integrated layers or membranes for analyte recovery.

In some embodiments, the plurality of integrated layers or membranes are treated such that a first portion of the integrated layers or membranes is configured to stabilize whole blood cells and a second portion of the integrated layers or membranes is configured to stabilize plasma.

In some embodiments, the apparatus further comprises a sensor for detecting an amount of sample collected, wherein the sensor comprises a biological sensor, a chemical sensor, or an optical sensor.

In some embodiments, the apparatus further comprises one or more geometric features disposed on at least a portion of the elongated strip, wherein the one or more geometric features are configured to provide a channel or flow path for the sample.

In some embodiments, the one or more geometric features comprise one or more relief features configured to (i) prevent overflow of a sample to a portion of the elongated strip, (ii) prevent hemolysis by (a) slowing one or more blood cells from intruding upon a plasma region of the elongated strip and (b) squeezing out or separating plasma from a whole blood sample, or (iii) provide physical separation of the different collection regions of the elongated strip for analysis of a plurality of analytes.

In some embodiments, the one or more geometric features are configured to provide a mechanical force or pressure to squeeze or separate plasma from a whole blood sample.

In some embodiments, the one or more geometric features comprise one or more notches configured to stop or near-stop sample flow to isolate plasma across one or more regions of the elongated strip.

In some embodiments, the elongated strip is operably coupled to a cartridge.

In some embodiments, the cartridge is configured to be coupled to a blood collection device.

In another aspect, disclosed is a system for analyzing a sample, comprising: the apparatus; and a cartridge, wherein the elongated strip is coupled to and/or inserted within the cartridge.

In some embodiments, the cartridge containing the elongated strip therein is configured to be operatively coupled to a blood collection device.

In another aspect, disclosed is a method, comprising: (a) providing an elongated strip having a dimensional aspect ratio of at least about 1:3 to about 1:10, wherein the elongated strip facilitates collection and processing of a sample; and (b) providing the sample to the elongated strip such that the sample flows along the elongated strip and separates into a first sub-sample comprising whole blood cells and a second sub-sample comprising plasma.

In some embodiments, the method further comprises, prior to (b), collecting the sample using an integrated blood collection device.

In another aspect, disclosed is a cartridge assembly comprising: an inlet component comprising a port configured to receive a blood sample; an elongated strip comprising a matrix configured to separate and collect plasma from the blood sample; a backing plate configured to couple to the inlet component and secure a proximal portion of the elongated strip between the inlet component and the backing plate; and an elongated housing configured to releasably couple to the inlet component, the elongated housing comprising an enclosure for receiving the elongated strip.

In some embodiments, the port comprises a tapered profile.

In some embodiments, an angle of the tapered profile ranges from about 0 degrees to about 45 degrees.

In some embodiments, a diameter of the port varies along a length of the port.

In some embodiments, a diameter at a distal end of the port is less than a diameter at a proximal end of the port.

In some embodiments, the inlet component comprises one or more turn features that are configured to induce a change in direction of flow of the blood sample for counteracting gravitational force on the flow.

In some embodiments, the one or more turn features are configured to cause the blood sample to flow onto the elongated strip in a first direction that is orthogonal to a second direction parallel to a flow of the blood sample through the port.

In some embodiments, the first direction is different from a direction of the gravitational force.

In some embodiments, the inlet component comprises a reservoir that is configured to collect, aggregate, or pool a volume of the blood sample as wicking of another portion of the blood sample occurs along the matrix.

In some embodiments, the reservoir is located adjacent to one or more turn features.

In some embodiments, the one or more turn features are located between the port and the reservoir.

In some embodiments, the inlet component comprises a pressure bar that is configured to regulate a flow speed of the blood sample and ensure proper wicking of the blood sample along the matrix for optimal separation of the plasma from the blood sample.

In some embodiments, the pressure bar is located adjacent to a reservoir.

In some embodiments, the reservoir is located between the pressure bar and one or more turn features.

In some embodiments, the inlet component comprises an indication window that is configured to permit a user to view a progress of the blood plasma separation on the matrix.

In some embodiments, the inlet component comprises a seal vent that permits vacuum pressure to equalize throughout and within and the cartridge assembly.

In some embodiments, the backing plate comprises a matrix vent.

In some embodiments, the backing plate comprises one or more spacers that are configured to create a gap between the inlet component and the backing plate.

In some embodiments, the gap is configured to be used in part with a pressure bar on the inlet component to regulate a flow speed of the blood sample and ensure proper wicking of the blood sample along the matrix.

In some embodiments, the backing plate comprises one or more guide features that are configured to guide and align the cartridge assembly for installation onto or with a blood collection device.

In some embodiments, the one or more guide features comprise a pair of guide rails that are laterally spaced apart on the backing plate.

In some embodiments, the enclosure is fully enclosed.

In some embodiments, the elongated housing comprises a seal that is configured to hermetically seal the enclosure.

In some embodiments, the seal extends along an opening of the elongated housing.

In some embodiments, the matrix comprises a glass fiber matrix.

In some embodiments, the matrix is treated.

In some embodiments, the matrix is untreated.

In some embodiments, the elongated strip further comprises a substrate on which the matrix is supported.

In some embodiments, the matrix is attached to the substrate using an adhesive.

In some embodiments, the substrate comprises an inert biocompatible material.

In some embodiments, the inert biocompatible material comprises mylar.

In some embodiments, the elongated strip further comprises a liner disposed between and separating the substrate and the matrix.

In some embodiments, the liner extends completely between the substrate and the matrix.

In some embodiments, the liner extends between the substrate and the matrix in a first region and does not extend between the substrate and the matrix in a second region that is different from the first region.

In some embodiments, the first region comprises a central portion of the elongated strip, and the second region comprises one or more end portions of the elongated strip.

In some embodiments, the first region comprises one or more end portions of the elongated strip, and the second region comprises a central portion of the elongated strip.

In some embodiments, a ratio of a length to a width of the elongated strip is about 2.3:1 to about 7:1.

In some embodiments, a length of the elongated strip is at least about 2.3 times greater than a width of the elongated strip.

In some embodiments, a length of the elongated strip is about 4.7 times greater than a width of the elongated strip.

In some embodiments, a length of the elongated strip is about 70% to 90% of a total length of the fully assembled cartridge assembly.

In some embodiments, a length of the elongated strip is about 85% of a total length of the fully assembled cartridge assembly.

In some embodiments, a distance from a distal end of the port to a proximal end of the elongated strip is about 0 mm to about 15 mm.

In some embodiments, a distance from a distal end of the port to a proximal end of the elongated strip is about 10 mm.

In some embodiments, a distance from a distal end of the port to a distal end of the elongated strip is about 35 mm to about 115 mm.

In some embodiments, a distance from a distal end of the port to a distal end of the elongated strip is about 75 mm.

In some embodiments, a distance from an edge of the reservoir to the pressure bar is about 0 mm to about 5 mm.

In some embodiments, a distance from an edge of the reservoir to the pressure bar is about 0 mm.

In some embodiments, a volume of the reservoir is about 30 mm³ to about 300 mm³.

In some embodiments, a volume of the reservoir is about 175 mm³

In some embodiments, a length of the reservoir is about 25% to about 75% of a width of the reservoir.

In some embodiments, a length of the reservoir is about 50% of a width of the reservoir.

In some embodiments, an edge of the elongated strip extends into the reservoir.

In some embodiments, an edge of the elongated strip extends to and is substantially aligned with an edge of the reservoir.

In some embodiments, a ratio of a width to a length of the pressure bar is about 5:1 to about 14:1.

In some embodiments, a width of the pressure bar is at least 5 times greater than a length of the pressure bar.

In some embodiments, a width of the pressure bar is about 7 times greater than a length of the pressure bar.

In some embodiments, an edge of the elongated strip extends to and is substantially aligned with the pressure bar.

In some embodiments, an edge of the elongated strip is at a distance of about 0 mm to about 10 mm from the pressure bar.

In some embodiments, an edge of the elongated strip extends by about 0 mm to about 10 mm beyond the pressure bar towards the reservoir.

In some embodiments, an edge of the elongated strip extends beyond the pressure bar into the reservoir by a distance of about 0 mm to about 10 mm from the pressure bar.

In some embodiments, the pressure bar is located at a distance of about 30 mm to about 90 mm from a distal end of the elongated strip such that the pressure bar is located along the elongated strip.

In some embodiments, an edge of the elongated strip does not extend beyond the pressure bar into the reservoir.

In some embodiments, a size of the gap is about 0 mm to about 4 mm.

In some embodiments, the pressure bar comprises the gap.

In some embodiments, a size of the gap is adjustable.

In some embodiments, a size of the gap is fixed.

In some embodiments, a size of the gap is substantially constant across a width or length of the gap.

In some embodiments, a size of the gap is variable across a width or length of the gap.

In some embodiments, a blood plasma separation performance of the matrix is improved by at least about 5×% with use of the pressure bar compared to without the use of the pressure bar.

In some embodiments, a blood plasma separation performance of the matrix is improved by at least about 5% when a length of the elongated strip is about 4.7 times greater than a width of the elongated strip.

In some embodiments, a blood plasma separation performance of the matrix is optimized when a length of the elongated strip is about 4.7 times greater than a width of the elongated strip.

In some embodiments, a blood plasma separation performance of the matrix is improved by at least about 5% with use of the seal vent compared to without the use of the seal vent.

In some embodiments, a blood plasma separation performance of the matrix is improved by at least about 5% with use of the matrix vent compared to without the use of the matrix vent.

In some embodiments, a ratio of an area of the cartridge assembly to an area of the elongated strip is about 1.5:1 to 2:1.

In some embodiments, the ratio is about 1.8:1.

Additional aspects and advantages of the present disclosure will become readily apparent to those skilled in this art from the following detailed description, wherein illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a perspective view of a sample acquisition device, in accordance with some embodiments;

FIG. 1B shows a perspective view of the cover of the sample acquisition device of FIG. 1A, in accordance with some embodiments;

FIG. 1C shows a perspective view of the lancing assembly of the sample acquisition device of FIG. 1A, in accordance with some embodiments;

FIG. 1D shows a perspective view of the cartridge assembly of the sample acquisition device of FIG. 1A, in accordance with some embodiments;

FIG. 1E shows a perspective view of the vacuum chamber and deposition chamber of the sample acquisition device of FIG. 1A, in accordance with some embodiments;

FIG. 2A shows a perspective view of a transport sleeve, in accordance with some embodiments;

FIG. 2B shows a cartridge assembly inserted into the transport sleeve, in accordance with some embodiments;

FIG. 3A shows different perspective views of a cartridge assembly, in accordance with some embodiments;

FIG. 3B shows a side sectional view of the cartridge assembly, in accordance with some embodiments;

FIG. 3C shows the side section view of the cartridge assembly with indications of sample flow directions, in accordance with some embodiments;

FIG. 3D shows a side sectional view of a sample acquisition device operatively coupled to the cartridge assembly, in accordance with some embodiments;

FIGS. 3E and 3F schematically illustrate cross-sectional views of another cartridge assembly and an exemplary use thereof, in accordance with some embodiments;

FIG. 4 shows a side section view of a different cartridge assembly with indications of sample flow directions, in accordance with some embodiments;

FIG. 5A shows a side sectional view (left) and a perspective view (right) of a sample chamber configured to collect liquid or liquid-like samples, in accordance with some embodiments;

FIG. 5B shows side sectional views of a sample acquisition device operatively coupled to the cartridge assembly, in accordance with some embodiments;

FIG. 5C shows perspective views of a visual metering window of a sample acquisition device operatively coupled to the cartridge assembly, in accordance with some embodiments;

FIG. 6 shows a cartridge assembly inserted into a transport sleeve, in accordance with some embodiments;

FIG. 7A shows perspective views (left two views) and a side sectional view of a modular sample chamber assembly for sample collection and storage, in accordance with some embodiments;

FIG. 7B illustrates principles of operation and use of a sample acquisition device operative coupled to the modular sample chamber assembly, in accordance with some embodiments;

FIG. 7C illustrates perspective views the sample acquisition device operative coupled to the modular sample chamber assembly, in accordance with some embodiments;

FIG. 7D shows different types of the modular sample chamber assembly for sample collection and storage, in accordance with some embodiments;

FIGS. 8A-8C illustrates multiple perspective views of a modular sample acquisition device and a modular sample chamber assembly, in accordance with some embodiments;

FIGS. 8D and 8E illustrate principles of operation and use of the modular sample acquisition device and the modular sample chamber assembly, in accordance with some embodiments;

FIG. 9 illustrates an example of a modular sample acquisition device operatively coupled to different types of modular sample chamber assemblies, in accordance with some embodiments; and

FIG. 10 shows example dimensional and pressure parameters of the devices for a sample acquisition process, in accordance with some embodiments.

FIG. 11 shows a perspective view of a sample acquisition device and cartridge assemblies in accordance with some embodiments.

FIG. 12 shows a perspective view of various components of a cartridge assembly, in accordance with some embodiments.

FIG. 13A shows a perspective view of various components of a blood filtration assembly, in accordance with some embodiments.

FIGS. 13B and 13C illustrate perspective views of the various components of the blood filtration assembly, in accordance with some embodiments.

FIG. 14 illustrates data analyses performed on samples collected from a sample acquisition device, in accordance with some embodiments.

FIG. 15 illustrates perspective views of various components of a blood separation assembly, in accordance with some embodiments.

FIG. 16A shows a perspective view of a first assembly structure of a blood separation assembly, in accordance with some embodiments.

FIG. 16B shows perspective views and a side sectional view of a blood separation assembly, in accordance with some embodiments.

FIG. 17A shows a perspective view and sectional views of a blood separation assembly, in accordance with some embodiments.

FIGS. 17B through 17D illustrate side sectional views of a blood separation assembly incorporating absorbent pads, in accordance with some embodiments.

FIGS. 17E through 17G illustrate perspective views of various components of the blood separation assembly, in accordance with some embodiments.

FIG. 18 shows perspective views of a treatment/stabilization unit for use in accordance with some embodiments.

FIG. 19 illustrates perspective views of a cartridge, in accordance with some embodiments.

FIG. 20 illustrates a perspective and side sectional views of a cartridge assembly which can provide visual cues to a user, in accordance with some embodiments.

FIGS. 21A through 21C illustrate sectional views of a blood separation assembly incorporating a releasing mechanism, in accordance with some embodiments.

FIG. 22 illustrates an exemplary matrix comprising one or more regions for stabilizing a fluid sample.

FIGS. 23A-F illustrate various examples of matrices with different geometries.

FIGS. 24A-E illustrate various examples of matrices with different geometric features such as notches and laser etched perforations.

FIGS. 25A-D illustrate various examples of matrices that are processed using different pretreatments.

FIGS. 26A-C illustrate a plasma cartridge assembly, in accordance with some embodiments.

FIGS. 27A-C illustrate different perspective views of a plasma cartridge assembly, in accordance with some embodiments.

FIGS. 28A-E illustrate perspective views of a cartridge port of a plasma cartridge assembly, in accordance with some embodiments.

FIGS. 29A-B illustrate perspective views of a matrix of a plasma cartridge assembly, in accordance with some embodiments.

FIGS. 30A-C illustrate a cartridge port of a plasma cartridge assembly, in accordance with some embodiments.

FIGS. 31A-C illustrate a cartridge backer of a plasma cartridge assembly, in accordance with some embodiments.

FIGS. 32A-B illustrate a cartridge tab of a plasma cartridge assembly, in accordance with some embodiments.

FIGS. 33A-B illustrate a matrix of a plasma cartridge assembly, in accordance with some embodiments.

FIGS. 34A-D illustrate a plasma cartridge assembly in a sample acquisition device, in accordance with some embodiments.

FIGS. 35A-E illustrate plasma yield of a plasma cartridge assembly, in accordance with some embodiments.

FIGS. 36A-D illustrate plasma yield of a plasma cartridge assembly, in accordance with some embodiments.

### DETAILED DESCRIPTION

Reference will now be made in detail to exemplary embodiments of the disclosure, examples of which are illustrated in the accompanying drawings. Wherever possible, the same reference numbers will be used throughout the drawings and disclosure to refer to the same or like parts.

### I. General

Provided herein are devices, methods, and kits for collecting a fluid sample, e.g., from a subject's body. The fluid sample can be, for example, blood, e.g., capillary blood, drawn from penetrated skin of the subject. The devices disclosed herein can be handheld and user-activatable (e.g., activatable by the subject from whom the fluid sample is to be drawn, or a third party user who assists the subject in drawing the fluid sample from the subject), and suitable for use outside of traditional healthcare facilities, for example in homes, in remote locations, while a subject is traveling, etc. The devices can be portable and easy to use, and allow individuals to efficiently and reliably collect their own blood samples, without relying on trained healthcare personnel, and without requiring the individual to have any prior blood draw training experience. The devices, methods, and kits described herein can be minimally invasive and permit lower levels of pain (or perception of pain) in a subject relative to use of other devices, methods, and kits, which can improve the overall blood draw experience for the subject. The kits can be provided with the devices and instructions that guide users on how the devices can be used for blood sample collection and storage. The kits can include transport sleeves and pouches for shipping/transportation of one or more samples to one or more testing facilities. The one or more samples can be collected within one or more sample chambers or a portion thereof (e.g., one or more cartridges) during the shipping/transportation. Alternatively, the sample chambers or a portion thereof may not and need not require any transport sleeve/pouch for shipping/transportation of the cartridges. In some examples, a cartridge can be enclosed in a housing (e.g., the sample chamber) that is configured to protect the cartridge during the shipping/transportation. The housing can be a tube, for example, as described and illustrated in some of the embodiments herein. The housing can permit treatment and/or stabilization of the sample prior to or during the shipping/transportation (e.g., the housing can comprise a desiccant). The cartridge can permit treatment and/or stabilization of the sample prior to or during the shipping/transportation. The housing can protect the collected sample from the external environment (e.g., controlling temperature, pressure, humidity, movement (e.g., vibration), etc.). In some cases, the housing can include a seal (e.g., a cap or a sealant) to prevent tampering of the collected sample that is stored inside the housing, prior to retrieval of the collected sample by a technician or medical professional for testing the collected sample. In some examples, the housing (or the sample chamber) can be secured by a physical lock that can be opened by a physical key or a digital key (e.g., by providing a digital key code).

Further provided herein are systems (e.g., devices), methods, and kits for treating (or processing) and/or storing the collected sample, e.g., fluid sample, in one or more of a plurality of different states comprising a liquid state, a semi-solid state, or a solid-state (e.g., dried state or solidified state). In some embodiments, blood, e.g., capillary blood, can be collected from a subject, and the collected blood can be processed and/or stored in one or more of a plurality of different formats comprising plasma, serum, dried blood, liquid blood, or coagulated blood.

A cartridge can be configured to support one or more matrices that are configured to hold at least a portion (e.g., at least a predefined volume) of collected blood. The cartridge can be configured to separate (e.g., isolate or filter) one or more components of the blood comprising plasma, serum, cells (e.g., leukocytes (or white blood cells) and/or erythrocytes (or red blood cells)), polypeptide molecules (e.g., proteins, such as growth factors), polynucleotide molecules (e.g., DNA, RNA, cell-free DNA (cfDNA), cell-free RNA (cfRNA), etc.), ions, and/or small molecules (e.g., nutrients). The systems (e.g., the devices), methods, and kits disclosed herein can selectively separate any number of sample components including cells, plasma, serum, platelets, specific cell types, DNA (e.g., tumor cfDNA), RNA, protein, inorganic materials, drugs, or any other components. The systems, methods, and kits disclosed herein can also be configured to store any separated component of the blood (e.g., plasma, serum, etc.).

Samples (e.g., blood samples) collected using the systems (e.g., sample acquisition devices), methods, and kits described herein can be analyzed to determine a subject's physiological state, for detecting diseases, and for monitoring a health condition of the subject. An individual can rapidly evaluate his or her physiological status, since samples (e.g., blood samples) can be quickly collected using the disclosed devices, methods, and kits, and the samples (e.g., blood samples) can be either (1) analyzed on the spot using, for example, immunoassays or (2) shipped, e.g., shipped promptly, to a testing facility. The reduced lead-time for blood collection, analysis and quantification can be beneficial to many users, e.g., subjects who have certain physiological conditions/diseases that require constant and frequent blood sample collection/monitoring.

Various systems (e.g., devices), methods, and kits of the present disclosure can be combined or modified with other systems, methods, and kits, such as, for example, those described in U.S. Patent Publication No. 2019/0000365 titled “DEVICES, SYSTEMS, AND METHODS FOR SAMPLE COLLECTION” and U.S. Patent Publication No. 2017/0067803 titled “SYSTEMS, METHODS, AND DEVICES FOR SAMPLE COLLECTION, STABILIZATION AND PRESERVATION,” each of which is incorporated herein by reference in its entirety.

Various aspects of the devices, methods, and kits described herein can be applied to any of the particular applications set forth herein and for any other types of fluid sample devices, in addition to blood collection devices. The devices, methods, and kits can be used in any system that requires a fluid sample to be drawn from the subject's body. It shall be understood that different aspects of the devices, methods, and kits described herein can be appreciated individually, collectively, or in combination with each other.

### I. Sample Acquisition Devices

A sample acquisition device as provided herein can be designed, configured, or used for collecting, treating (e.g., separating), storing, and/or stabilizing at least a portion of a sample, e.g., a fluid sample, e.g., a fluid sample drawn from a subject. The sample can be a biological sample. The biological sample, or fluid sample, can be whole blood, blood serum, blood plasma, or the like. The sample acquisition devices can be configured to be held and operated by a user's hand. The user can be the subject or a third party, e.g., a medical practitioner. A sample acquisition device can be handheld (e.g., by one or two hands of the user, by multiple hands from multiple users such as the subject and the medical professional, etc.) during use. Thus, any sample acquisition device of the present disclosure can be a handheld device.

The sample acquisition device provided herein can be used in a variety of locations or environments or applications including, e.g., at the subject's own home, at a remote location, on-site or while the subject is traveling, for personalized healthcare, in a point-of-care (POC) setting, at a hospital, at a clinic, at an emergency room, at a patient examination room, in an acute care patient room, in ambulatory care, in the field of pediatrics, in a field environment, at a nurse's office in an educational setting, at an occupational health clinic, during surgery or in an operation room.

The sample acquisition device can be used to collect and store a sample (e.g., blood) drawn from a subject. The subject can be a patient. The subject can be an animal, e.g., a primate or a non-primate. The subject can be a male or female. The subject can be pregnant, suspected of being pregnant, or planning to become pregnant. The subject can be ovulating. The subject can have, or be suspected of having, a condition, e.g., cancer, autoimmune disease, or diabetes. The subject can be a human, and the human can be an infant, child, teenager, adult, or elderly person.

The sample acquisition device, (devices can be easily and conveniently used by the subject to draw the sample, e.g., blood sample, from the subject without the help or aid of a third party. In some cases, the device can be operated by a third party to collect blood from the subject. The third party can include, for example, a family member of the subject, a medical professional, for example, physician, nurse, or an Emergency Medical Technician (EMT), a clinician, or a laboratory technician. The third party can be a non-living entity, e.g. a robot.

The sample acquisition device can be designed such that it is minimally invasive and generates a low level of pain (or reduced perception of pain) in the subject. For example, the sample acquisition device can include a low number (e.g. one or two) piercing elements, instead of an array of multiple (three, four, five or more) needles or microneedles for penetrating the skin. The device need not be pre-packaged with one or more piercing elements. For example, a variety of piercing elements can be operably and releasably coupled to the device, and/or interchanged onto the device e.g., after each use. In some alternate cases, the device can be operated without using piercing elements. For example, a subject's skin can have one or more pre-existing cuts, and the device can be placed over the one or more pre-existing cuts to draw blood using skin suction and vacuum pressure.

The device can be portable, disposable and designed for use in a single subject encounter. In any of the embodiments disclosed herein, the device can be re-usable. For example, a device can be used more than once, for example twice, three, four, five, five, six, seven, eight, nine, ten or more times. In any of the embodiments disclosed herein, a single device can be used in multiple subject encounters, either with a same subject or with a plurality of different subjects. The device can be of a form factor and ergonomically designed to facilitate the sample collection process. Sample collection, treatment and storage can be performed on a single device. In some cases, sample collection, treatment and storage can be performed using multiple components or devices (e.g., a piercing module and a vacuum module can be provided as separate devices that are operably connected or coupled together via one or more channels).

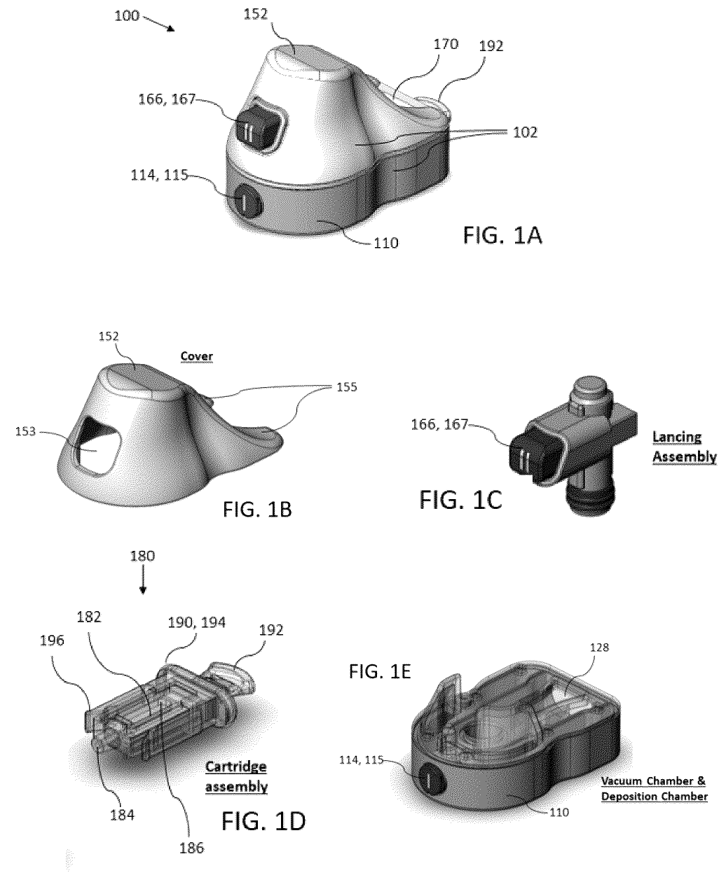

FIGS. 1A and 1B illustrate an exemplary device 100, in accordance with some embodiments. Specifically, FIG. TA shows an overall perspective view of the device. The device can include a housing 102. The housing can include a housing base 110 and a housing cover 152 operably coupled to each other. In some embodiments, the housing base 110 can encompass a vacuum chamber and a deposition chamber as described further herein.

In any of the embodiments disclosed herein, a housing can be provided separately from the components of the device, and the housing need not be part of or integrated with the components. For example, a vacuum chamber, deposition chamber, cartridge chamber, and/or cartridge assembly (or cartridge module) as described elsewhere herein can be operably coupled to a separately provided housing. A recess as described herein can be provided on a portion of the housing. A housing can include a casing, enclosure, shell, box, and the like. A housing can include one or more hollow chambers, cavities or recesses. The housing can be formed having any shape and/or size. The housing can be configured to support one or more components as described elsewhere herein. Additionally, one or more of the components can serve or function as the housing. The housing can be integrated with one or more of the components herein, or one or more of the components can be integrated with or into the housing. The housing can be configured for mounting onto a surface such as, for example, skin of a subject. In any of the embodiments disclosed herein, a bracket or strap can be provided that allows the housing to be mounted to a surface.

The device can include a vacuum activator 114. The vacuum activator can include a button 115 located on the housing base. In some cases, the device does not have a vacuum activator or need not have a vacuum activator. In an example, installation of a sample chamber into the device can automatically provide a vacuum in the vacuum chamber of the device. The device can further include a piercing activator 166. The piercing activator can include a button 167. In some cases, the button 167 can be disclosed adjacent to the housing cover. In some cases, the device does not have a piercing activator or need not have a piercing activator. In an example, the device can be used to draw blood from skin that has already been penetrated or pre-cut by other discrete stand-alone piercing elements. In another example, installation of a sample chamber into the device can automatically activate the device to pierce the skin of the subject. The piercing can be preferably activated (e.g., via the piercing activator) after the vacuum activator has been activated. In some cases, the piercing can be activated independently of the vacuum activator or vacuum state of the device. In some embodiments, the piercing activator can be locked prior to use of the device, and the piercing activator can be activated only after the vacuum activator has been activated. In some cases, the vacuum activator is locked prior to use of the device, and the vacuum activator can be activated only after the piercing activator has been activated. As abovementioned, the device 100 or any of the devices herein can be operatively coupled to a sample chamber, e.g., a cartridge assembly 180 as illustrated in FIG. 1A. Such cartridge assembly can be releasably coupled to the device and detached from the device. A cartridge tab 192 of the cartridge assembly can protrude from an edge of the device. In any of the embodiments disclosed herein, the cartridge tab and the piercing activator/vacuum activator (e.g., buttons 115/167) can be located on different sides (e.g. opposite ends) of the housing. Additional details about the vacuum activator and the piercing activator are described herein.

Sample acquisition devices for collecting a blood sample can be modular (i.e., “modular devices”), with two or more components for performing specific actions or functions on the device. FIG. 1B shows various components and subassemblies of a modular sample acquisition device 100. The modular device can comprise a plurality of modules (or subassemblies). An individual module can be a replaceable or swappable unit. In some cases, after a single use of the modular device, an individual module of the device can be replaced with a new module while one or more other modules of the device can be reusable. One or more modules of the modular device can be reusable for at least 1, 2, 3, 4, 5, 6, 7, 8, 9, 10 or more uses of the device. In comparison to a non-modular device (e.g., cannot be easily broken down into a plurality of components), the modular device can comprise one or more benefits, such as ease of partial replacement, partial maintenance or repair, partial upgrade, cleaning, a reduced cost of manufacturing or packaging, etc. In some embodiments, as shown in FIG. 1B, the modular device can comprise (1) a housing cover 152 that comprises a through-hole 153 through which the button 167 of the piercing activator 166 can be inserted, (2) a lancing assembly that comprises the button 167 of the piercing activator 166, and (3) a housing base 110 that comprises the vacuum activator 114 (e.g., the button 115). In some cases, the housing base 110 can serve as a vacuum chamber and/or a deposition chamber. In some cases, the lancing assembly can comprise a lancing mechanism to pierce the skin of the subject (e.g., through an opening of the housing base 110). The lancing assembly can be configured to activate a lancing mechanism disposed within the housing base 110 to pierce the skin of the subject.

FIG. 1B further shows a sample chamber configured to be operatively coupled to the modular device. For example, the sample chamber can be a cartridge assembly 180 that can be releasably coupled to the modular device. The cartridge assembly can be part of the modular device, and can be decoupled from the device. The cartridge assembly can be inserted into a deposition chamber (or cartridge chamber) of the housing base of the modular device via an opening 128. The cartridge assembly can include a cartridge 182 and a cartridge holder 190. The cartridge holder can be configured to support the cartridge. The cartridge holder can include a cartridge tab 192, a seal/gasket 194, and spring clips 196. A user (e.g., a subject) can handle or hold the cartridge assembly using the cartridge tab. For example, the subject can insert the cartridge assembly into the deposition chamber (cartridge chamber) of the modular device by pushing in the cartridge tab. After the sample collection has been completed, the subject can remove the cartridge assembly from the deposition chamber (cartridge chamber) of the modular device by pulling the cartridge tab. The subject can also hold the cartridge assembly by the cartridge tab to avoid contamination to the cartridge and/or sample. The seal/gasket 194 can hermetically seal the deposition chamber (cartridge chamber) once the cartridge assembly is properly inserted into the modular device. The spring clips 196 allow the cartridge to be held in place by the cartridge holder.

The cartridge 182 of the cartridge assembly can be configured to support one or more matrices 186 on which the fluid sample (e.g., blood) is collected. In some embodiments, the cartridge can support two or more matrices. The two or more matrices can be separated by one or more spacers. The cartridge can include a cartridge port 184 and a channel (not shown) leading to the matrices. The cartridge can be configured to support one or more absorbent pads (not shown) for holding excess fluid. The absorbent pads help to ensure that a predefined volume of fluid can be collected on each of the matrices. Additional details about the cartridge assembly are described, e.g., in Section II Part C of the Specification.

The housing base 110 and the housing cover 152 can each be separately provided, and coupled together to form the housing. The housing base can include a vacuum chamber and a deposition chamber. The vacuum chamber and the deposition chamber can be separated by one or more walls. The walls can be substantially impermeable to fluids (e.g., gases and liquids). The lid can hermetically seal the vacuum chamber and the deposition chamber. The lid can include a flow meter. The deposition chamber can also serve as a cartridge chamber and can be interchangeably referred to as such herein. The cartridge assembly 180 can be inserted into the deposition chamber (or cartridge chamber). The seal/gasket 194 can hermetically seal the deposition chamber once the cartridge assembly is fully inserted into the deposition chamber. The housing cover can include wings 155 having a U or V-like shape to prevent obscuring the flow meter on the lid of the housing base. Accordingly, the housing cover can be shaped in a manner that allows the user (e.g., the subject or third party user) to view the flow meter and monitor the progress of the fluid sample collection. The housing cover can have a vertical (Z-height) clearance that permits placement of a piercing module therein (e.g., which is to be a part of the lancing assembly as shown in FIG. 1B). The piercing module can comprise one or more piercing elements that are configured to extend and retract through the opening of the recess.

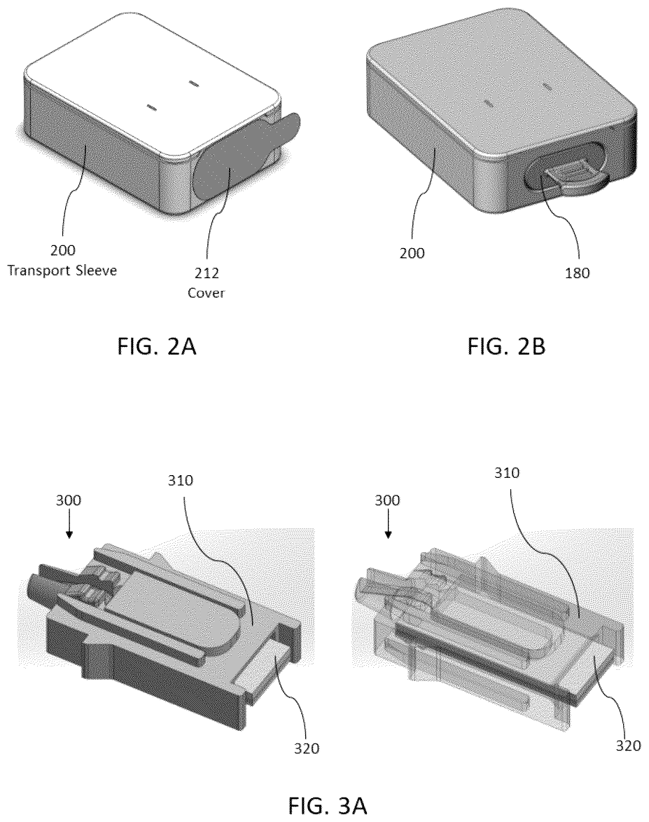

In alternative embodiments, the sample chamber (e.g., the cartridge assembly) or a component thereof that comprises the collected sample can be removed from the sample acquisition device and stored in a storage/transport device. FIG. 2A shows a perspective view of a transport sleeve 200 that can be used for packaging of a filled sample chamber or samples from within the sample chamber. The sleeve can include a hollow interior for storing the filled sample chamber or samples during shipment/transportation. The sleeve can include an opening for receiving the sample chamber (e.g., the cartridge assembly). In some embodiments, the sleeve can include a cover 212 (e.g., a peelable foil) for covering the opening prior to use of the sleeve. The cover 212 can be, for example a foil that can be attached to the opening via an adhesive, and peeled off by a user prior to use of the sleeve. A desiccant (not shown) can be disposed within the sleeve, and used for drying and/or keeping the samples dry. The foil cover can help to protect the interior of the sleeve from moisture and contamination prior to use. FIG. 2B shows a perspective view of the transport sleeve 200 subsequent to inserting the cartridge assembly 180 into the transport sleeve. Additional details about the cartridge assembly are described, e.g., in Section III of the Specification.

One or more components of the device or that are operatively coupled to the device (e.g., any one of the modules, any type of the sample chamber, the transport sleeve, etc.) can be formed having any shape and/or size. Such component(s) can be formed using any number of techniques known in the art such as injection molding, blow molding, three-dimensional (3D) printing, etc. Such components that are configured to contact the patient can include materials suitable for healthcare applications (e.g., the housing material is compatible for use with biological materials), depending on the particular application. For example, components of the housing of the sample acquisition device can include or be fabricated from materials such as copolyester (e.g., polyethylene terephthalate (PET), polyethylene terephthalate glycol (PETG), polypropylene, polycarbonate, cellophane, vinyl, acetate, polyethylene acrylic, butyl rubber, ethylene-vinyl acetate, natural rubber, a nitrile, silicone rubber, a styrene block copolymer, a vinyl ether, or a tackifier. In any of the embodiments disclosed herein, such component(s) can include antimicrobial and/or antiseptic materials, for example sodium bicarbonate; hydrogen peroxide; benzalkonium chloride; chlorohexidine; hexachlorophene; iodine compounds; and combinations thereof.

In any of the embodiments disclosed herein, one or more components of the device or operatively coupled to the device (e.g., any one of the modules, any type of the sample chamber, the transport sleeve, etc.) can include or can be fabricated from materials such as polyvinyl chloride, polyvinylidene chloride, low density polyethylene, linear low density polyethylene, polyisobutene, poly[ethylene-vinylacetate]copolymer, lightweight aluminum foil and combinations thereof, stainless steel alloys, commercially pure titanium, titanium alloys, silver alloys, copper alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, partially resorbable materials, such as, for example, composites of metals and calcium-housing based ceramics, composites of PEEK and calcium housing based ceramics, composites of PEEK with resorbable polymers, totally resorbable materials, such as, for example, calcium housing based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

One or more components of the device or that are operatively coupled to the device (e.g., any one of the modules, any type of the sample chamber, the transport sleeve, etc.) can have material composites, including one or more of the above materials, to achieve various desired characteristics such as strength, rigidity, elasticity, compliance, biomechanical performance, durability and/or radiolucency preference. Such components, individually or collectively, can also be fabricated from a heterogeneous material such as a combination of two or more of the above-described materials. The components of the device can be monolithically formed or integrally connected.

One or more components of the device or that are operatively coupled to the device (e.g., any one of the modules, any type of the sample chamber, the transport sleeve, etc.) can be ergonomically designed such that a user (e.g., the subject) is able to hold and/or operate the device and/or the sample chamber comfortably with one hand or both hands. The device can have a compact form factor that makes it highly portable (e.g., easy to be carried around in a user's bag or purse). Exemplary dimensions (e.g., length, width and height) of the sample chamber are described elsewhere herein.

### A. Recess for Skin Suction

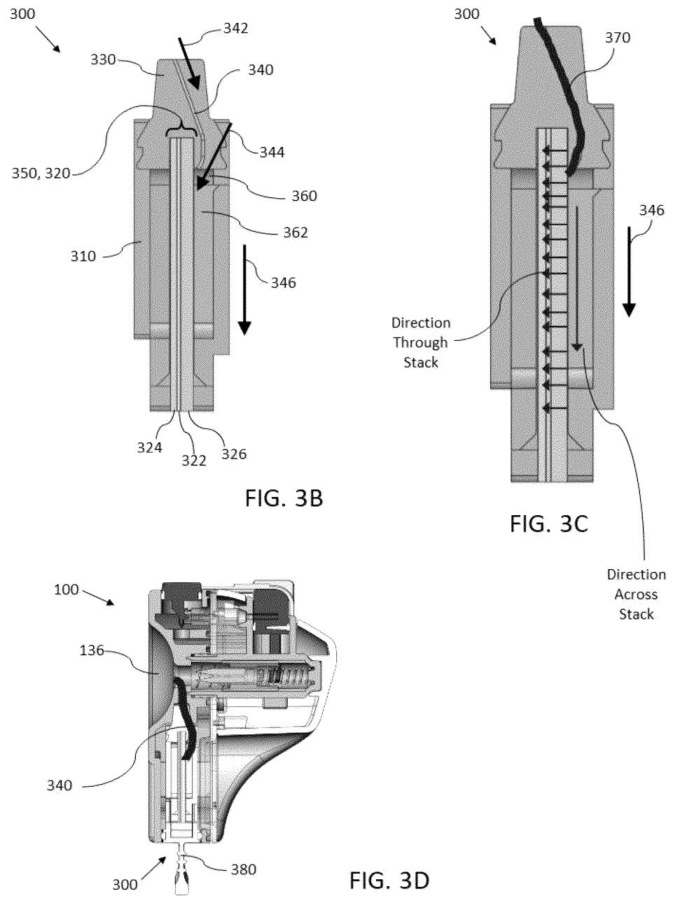

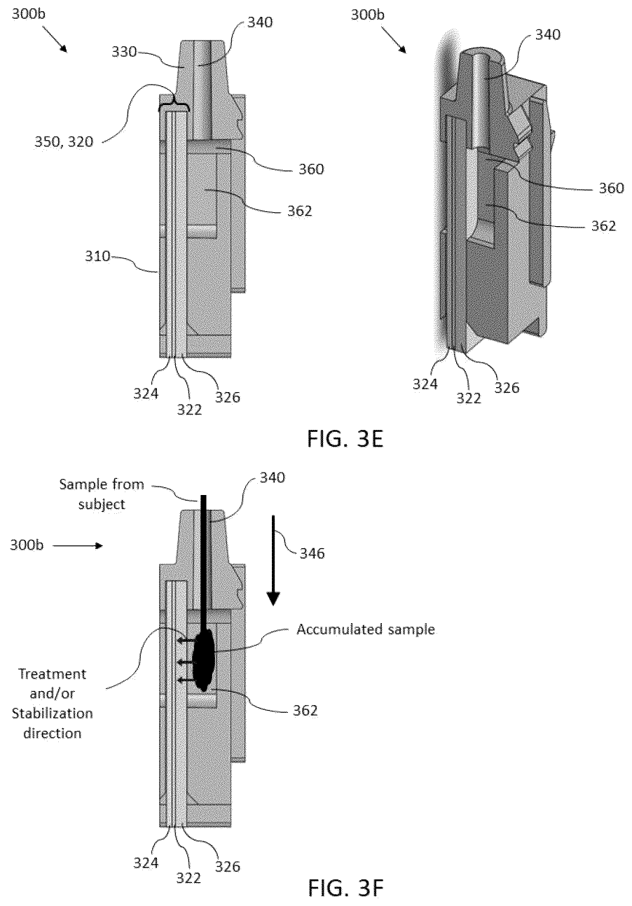

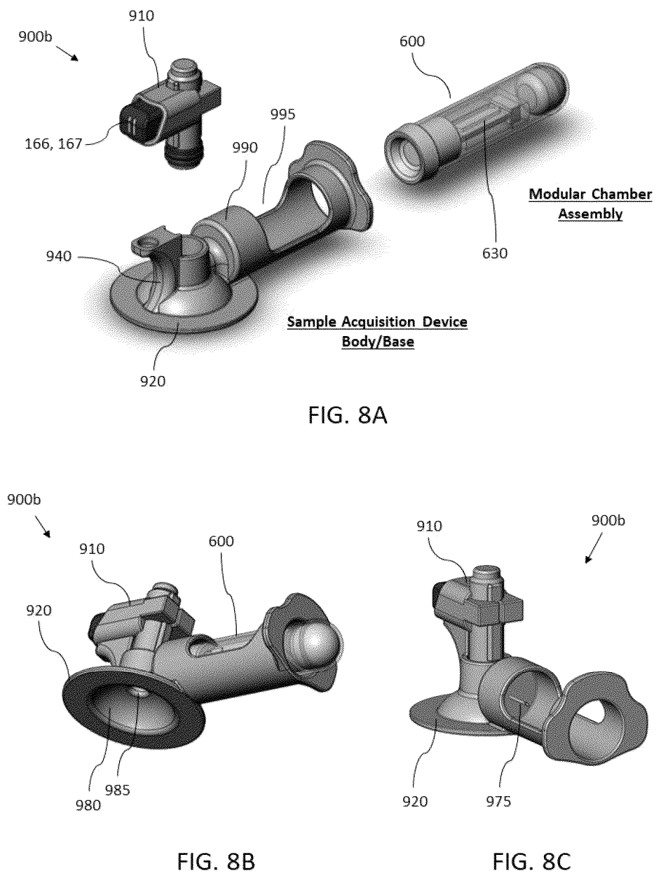

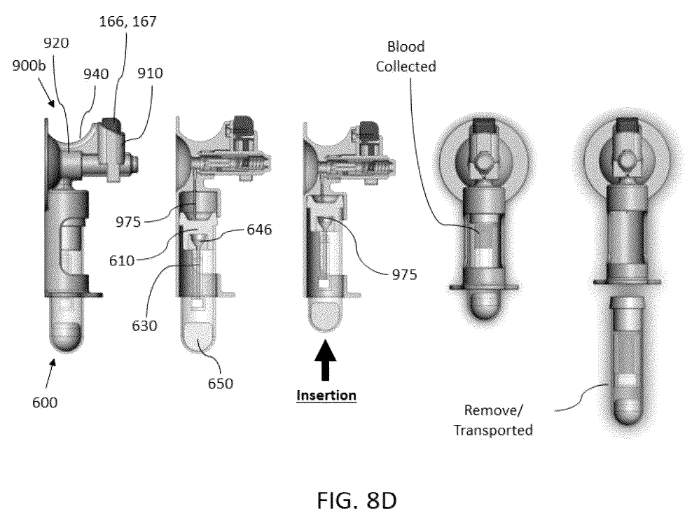

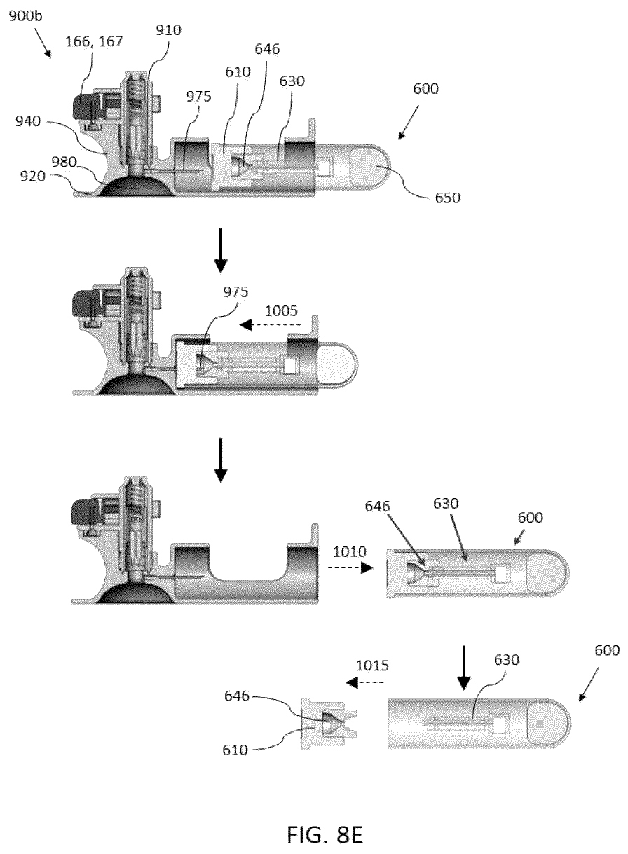

In some embodiments, the housing base 102 of the device can include a recess 136 (as shown in FIG. 3D and FIG. 8C). The recess can be provided on a portion (e.g., bottom surface) of the housing base. The recess can be formed as a sunken cavity or trench on the housing base. In some cases, the recess can be formed as a molded extrusion into the housing base. The recess can be shaped like a cup and configured to provide a skin “cupping” effect with aid of vacuum pressure. The recess can be sized and/or shaped to receive a portion of a surface, e.g., subject's skin therein, and to permit the surface, e.g., skin to substantially conform to the recess under application of vacuum pressure. A surface of the recess can be substantially in contact with the skin drawn into the recess. A gap between the skin and the recess can be negligible when the skin is drawn into the recess. The recess can serve as a suction cavity for drawing the skin therein and for increasing capillary pressure differential.

In some alternative embodiments, the device can be configured to draw other types of objects (e.g. objects that are not skin or skin surfaces) into the recess under vacuum, and to further draw a fluid sample from those objects. Examples of biological samples suitable for use with the devices of the disclosure can include sweat, tears, urine, saliva, feces, vaginal secretions, semen, interstitial fluid, mucus, sebum, crevicular fluid, aqueous humour, vitreous humour, bile, breast milk, cerebrospinal fluid, cerumen, enolymph, perilymph, gastric juice, peritoneal fluid, vomit, and the like. In some embodiments, a fluid sample can be a solid sample that has been modified with a liquid medium. In some instances, a biological sample can be obtained from a subject in a hospital, laboratory, clinical or medical laboratory.

The recess of the device can be configured to maintain contact with a skin surface area of the subject under vacuum pressure, prior to and as blood is being collected from penetrated skin of the subject. In some embodiments, the volume of the enclosed within the recess can be substantially the same as an inner volume of the recess. In some embodiments, the recess can be configured to provide a safety feature. In an example, the lancet can be configured to protrude a short distance into the cavity of the device, such that a length of the protruded portion of the lancet is shorter than the height of the recess. Thus, upon the protrusion, a tip of the lancet may not be in contact with the subject's skin. The tip can come in contact with the skin upon suction of the skin towards the recess. Such feature can prevent cut(s) of the skin during undesired (e.g., accidental) actuation of the lancet or in absence of vacuum to suction the skin towards the recess.

### B. Vacuum Chamber and Deposition Chamber

The device can include a vacuum chamber and/or a deposition chamber. The vacuum chamber and the deposition chamber can be provided in the housing (e.g., integrated into the housing base). The vacuum chamber and the deposition chamber can be operably coupled to a separately provided housing or housing body (e.g., as illustrated in FIG. 1B). The vacuum chamber can be configured to be in fluidic communication with the recess and the deposition chamber. The vacuum chamber and the deposition chamber can be part of the housing base. The vacuum chamber and the deposition chamber can be located in different sections (e.g., compartments) of the housing base, and provided having various shapes or configurations. The vacuum chamber and the deposition chamber can be separated by one or more walls. In some alternative cases, the vacuum chamber and the deposition chamber need not be separated, e.g., by walls. For example, the vacuum chamber and the deposition chamber can be the same chamber in a device as packaged. The combined vacuum chamber and the deposition chamber can be a monolithic chamber.

The deposition chamber can be interchangeably referred to as a cartridge chamber and can be considered part of the sample acquisition device, since the deposition chamber can be configured to receive a sample chamber (e.g., the cartridge assembly 180) therein. For example, blood can be collected from the subject, and transported from the recess into the deposition chamber for collection and storage within the sample chamber, e.g., a cartridge of the cartridge assembly 180.

In some embodiments, a mechanical device such as a vacuum pump can be used to evacuate the vacuum chamber or similar chambers (e.g., before or after packaging). The mechanical device can include components such as pistons, motors, blowers, pressure regulators, venturis and the like. In some cases, non-mechanical means, such as chemicals or other reactants, can be introduced to the vacuum chamber and can undergo reaction to decrease pressure within the vacuum chamber (e.g., create a vacuum state). In other embodiments, the vacuum chamber may not and need not require a mechanical device to evacuate the vacuum chamber. For example, the sample chamber can be under vacuum, and installation of the sample chamber to the sample acquisition device (e.g., the device 100) can induce negative pressure in the device (e.g., the device body and/or the rest of the internal chambers and channels of the device).

The volume and flowrate of the blood collection by a system (e.g., a sample acquisition device) can depend on a starting or initial vacuum pressure of the vacuum chamber. The starting or initial vacuum pressure can correspond to the pressure of the vacuum chamber post evacuation. In some embodiments, the initial vacuum pressure of the vacuum chamber can range from about −4 pounds per square in gauge (psig) to about −15 psig (e.g., −14.7 psig at sea level), preferably about −8 psig to about −12 psig. In some preferred embodiments, the initial vacuum pressure of the vacuum chamber can be about −12 psig. In some other embodiments, the initial vacuum pressure of the vacuum chamber can be less than about −12 psig, e.g., about −13 psig or −14 psig.

In some embodiments, the device 100 or any other sample acquisition device disclosed herein can be configured to collect smaller amounts of blood (e.g. less than 150 microliter (μL), 140 μL, 130 μL, 120 μL, 110 μL, 100 μL, 90 μL, 80 μL, 70 μL, 60 μL, 50 μL, 40 μL, 30 μL, or 25 μL) of blood from a subject within a time window beginning from time of incision or penetration of a skin portion of the subject. In some embodiments, the device 100 or any other sample acquisition device disclosed herein can be configured to collect larger amounts of blood, e.g., at least 150 μL, 200 μL, 300 μL, 400 μL, 500 μL, 600 μL, 700 μL, 800 μL, 900 μL, 1,000 μL, 2,000 μL, 3,000 μL, 4,000 μL, 5,000 μL, or more. In some embodiments, the time window can be less than about 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, or less. In an example, the time window can be less than 5 minutes, preferably less than 3 minutes. In another example, the time window can be under 2 minutes. In a different example, the time window can be under one minute.

### C. Piercing Module

The sample acquisition device can include a piercing module for penetrating the skin of the subject when the skin is drawn into the recess under vacuum pressure. In some alternative cases, the device need not comprise a piercing module. In some embodiments, the piercing module can be provided in the lancing assembly module, as illustrated in FIG. 1B. The piercing elements can include lancets, lances, blades, needles, microneedles, surgical knives, sharps, rods, and the like. Any number of piercing elements (e.g., at least 1, 2, 3, 4, 5, 6, 7, 8, 9, 10 or more piercing elements) can be contemplated. In some embodiments, the piercing elements can preferably comprise two lancets.

The piercing module can further comprise one or more actuation elements (e.g., spring elements) for actuating the lancet holder and moving the piercing elements. Other non-limiting examples of actuation elements can include magnets, electromagnets, pneumatic actuators, hydraulic actuators, motors (e.g. brushless motors, direct current (DC) brush motors, rotational motors, servo motors, direct-drive rotational motors, DC torque motors, linear solenoids stepper motors, ultrasonic motors, geared motors, speed-reduced motors, or piggybacked motor combinations), gears, cams, linear drives, belts, pulleys, conveyors, and the like. Non-limiting examples of spring elements can include a variety of suitable spring types, e.g., nested compression springs, buckling columns, conical springs, variable-pitch springs, snap-rings, double torsion springs, wire forms, limited-travel extension springs, braided-wire springs, leaf springs etc. Further, the actuation elements (e.g., spring elements) can be made from any of a number of metals, plastics, or composite materials.

### D. Vacuum Activator and Piercing Activator

The device can include a vacuum activator 114 configured to activate the (evacuated) vacuum chamber, which generates a vacuum pressure that can draw the skin into the recess and subsequently facilitate collection of blood from the penetrated skin. The device can also include a piercing activator 166 configured to activate the deployment spring, for actuating the piercing elements. The vacuum activator can be separate from the piercing activator. For example, the vacuum activator and the piercing activator can be two separate discrete components of the device. In some alternative embodiments (not shown), the vacuum activator and the piercing activator can be integrated together as a single component that can be used to simultaneously or sequentially activate the vacuum and the piercing elements.

In some embodiments, the vacuum activator can be activated first, followed by the piercing activator. In other words, vacuum pressure can be activated prior to activation of the piercing elements. In certain embodiments, the piercing activator can be activated only after the vacuum activator and vacuum have been activated. For example, the piercing activator can be initially in a locked state, and incapable of activating the one or more piercing elements prior to activation of the vacuum. The piercing activator can be unlocked only after the vacuum activator has been activated. The above effect can be achieved by providing a locking mechanism that couples the piercing activator to the vacuum activator. The locking mechanism can be configured such that the piercing activator is initially in the locked state. The vacuum activator can function as a key for unlocking the piercing activator, and the piercing activator can be simultaneously unlocked when the vacuum activator is activated.

In some embodiments, the piercing activator can be configured to activate the one or more piercing elements after the skin is drawn into the recess. The piercing activator can be configured to activate the one or more piercing elements after the skin is drawn into the recess by the vacuum for a predetermined length of time. The predetermined length of time can be, for example, at least about 1 second, 5 seconds, 10 seconds, 20 seconds, 30 seconds, 40 seconds, 50 seconds, 60 seconds, or more. The predetermined length of time can be at most about 60 seconds, 50 seconds, 40 seconds, 30 seconds, 20 seconds, 10 seconds, 5 seconds, 1 second, or less.

In any of the embodiments disclosed herein, the vacuum activation can be semi-automatic or fully automatic. In some embodiments, the device need not require manual vacuum activation. For example, the device can be configured to automatically apply the vacuum upon sensing or detecting that the device has been placed on a surface (e.g., on a subject's skin), or that the recess of the device is properly placed over the surface. In any of the embodiments disclosed herein, activation of the piercing elements can be semi-automatic or fully automatic. For example, the piercing elements can be automatically activated to penetrate the surface (e.g., a subject's skin) upon sensing or detecting that the surface is drawn into the recess of the device, and/or that the surface is in proximity to the opening (e.g., 140) of the recess. The above sensing or detection (for the vacuum activation and/or piercing activation) can be enabled using any variety or number of sensors. The sensors can be included with the device (e.g., onboard the device) or remote from the device. Non-limiting examples of sensors that can be used with any of the embodiments herein include proximity sensors, tactile sensors, acoustic sensors, motion sensors, pressure sensors, interferometric sensors, inertial sensors, thermal sensors, image sensors, and the like. In some cases, if the vacuum activation and/or piercing activation is configured to be semi-automatic or fully automatic, the buttons for the piercing activator and/or piercing activator can be included (or omitted) from the device. In some embodiments, the device can be configured to automatically apply the vacuum upon a complete installation (e.g., insertion) of the cartridge assembly into the device (e.g., via vacuum venting from the cartridge assembly and towards the device).

### E. Sample Chamber

As previously described the sample acquisition device (e.g., the cartridge chamber of the device) can be configured to receive a sample chamber. The sample chamber can be a body configured to be operatively coupled to a sample acquisition device to receive, store, and/or treat at least a portion of a subject's sample. A sample chamber can used with one or more types of sample acquisition devices, as disclosed herein. For example, a sample chamber can be used interchangeably with a sample acquisition device 100 as shown in FIG. 1A and a modular sample acquisition device 900b in FIG. 8A. In some cases, the sample chamber can be container (e.g., a tube) to collect the subject's liquid sample (e.g., liquid blood). In some cases, the sample chamber can comprise one or more cartridges to collect other types or formats of the subject's sample (e.g., plasma or serum). In some examples, a sample chamber can be a cartridge assembly comprising a cartridge. The cartridge can comprise one or more matrices (e.g., one or more solid matrices) for sample collection and/or storage. In some embodiments, the sample chamber can comprise a cartridge assembly that is configured to hold one or more matrices for storing and/or treating a fluid sample (e.g., blood) thereon, and a cartridge holder for supporting the cartridge. The cartridge holder can be releasably coupled to the cartridge or other component(s) in the cartridge assembly, using, for example spring-clips. The cartridge assembly can be configured to releasably couple to the device 100 used for collecting blood from the subject. The cartridge holder can include a cartridge tab that is configured to be releasably coupled to a distal end of the cartridge chamber. The cartridge tab can be designed such that the user (e.g., the subject) is able to (1) support the cartridge assembly by holding the cartridge tab, (2) couple the cartridge assembly to the device by pushing in the cartridge tab, and/or (3) decouple the cartridge assembly from the device by pulling the cartridge tab. In alternative embodiments, the cartridge holder can be a part of the cartridge assembly, e.g., the cartridge holder can be a permanent part of the cartridge assembly and thus may not or need not be releasably coupled to the cartridge assembly.

The sample chamber can be coupled to the cartridge chamber prior to the collection of blood from the subject, and decoupled from the cartridge chamber after blood from the subject has been collected into at least a portion of the sample chamber. In some embodiments, the sample chamber can include one or more matrices for collecting, storing, and/or stabilizing the collected blood sample. The matrices can be provided in strip form (as strips). A strip as used herein can refer to a solid matrix that is sized and/or shaped to maximize blood collection volume while still fitting into commonly used containers (e.g., a 3 ml BD vacutainer, deep well plate or 2 ml Eppendorf tube). A matrix as used herein can be interchangeably referred to herein as a matrix strip, a strip, a solid matrix, a solid matrix strip, and the like.

In some embodiments, the matrices herein can also enable lateral transport/flow of the blood. Non-limiting examples of the matrices can include absorbent paper strips (e.g. cellulose fiber or 100% pure cotton linter filter paper), or a membrane polymer such as nitrocellulose, polyvinylidene fluoride, nylon, Fusion 5™, or polyethersulfone. In some embodiments, the matrices can comprise cellulose fiber based paper (e.g. Whatman™ 903 or Ahlstrom 226 paper), paper treated with chemicals or reagents for stabilizing the sample or one or more components of the sample (e.g., RNA stabilization matrix or Protein Stabilization Matrix). In some embodiments, the matrix comprises a cellulose filter paper. Any suitable commercially available filter paper can be used. Examples of commercially available filter paper include, but are not limited to, a glass fiber filter material, filter paper from Whatman®, such as 903 sample collection cards and fast transit analysis (FTA®) card. In some embodiments, the matrix can comprise a nitrocellulose filter paper. In some embodiments, the matrix does not or need not comprise any filter paper.

The collection of the fluid sample can be aided by the natural wicking or capillary action associated with the matrix, which can enhance and accelerate the absorption or collection of the fluid sample onto the matrix. In some cases, the matrices can be composed of a material comprising a plurality of capillary beds such that, when contacted with a fluid sample, the fluid sample is transported laterally across the matrices. The fluid sample fluid can flow along a flow path from a proximal end to a distal end of the matrices, for example by wicking or capillarity.

The sample chamber can comprise self-metering capability which can be advantageous for collecting a predefined volume of blood (e.g., into the sample container, into a cartridge of the sample chamber, etc.) for each individual person, regardless of varying input volumes of blood flow to the sample chamber for different individual persons. The variations in input blood volume into the sample chamber can occur since capillary pressures and blood flow can often vary from individual to individual (e.g., due to age, gender, health, etc.). Alternatively or in addition to, input blood volume into the sample chamber can vary due to handling of the operator (e.g., how fast or how well the sample chamber is coupled to the sample acquisition device) or the time it takes between (1) completion of sample collection into the sample chamber and (2) removal of the sample chamber or at least a portion thereof from the sample acquisition device. In some examples, the sample chamber can be a cartridge assembly comprising matrix strips, and the design of the cartridge assembly can ensure that matrix strips consistently contain a target blood volume independent of the volume of the blood that enters the cartridge (within or up to a predefined range). The cartridge assembly can further comprise one or more absorbent pads configured to absorb excess sample (e.g., excess blood) and enable the metering capabilities.

The sample chamber described herein can be used for collection of a sample (e.g., blood) from a subject. The sample chamber can be further configured for treatment, stabilization, and/or storage of the sample. In some cases, the sample chamber can store the sample (e.g., in liquid form, solid form, semi-solid form, etc.) and subsequently treat and/or stabilize the sample. Such treatment can be automatic or triggered by a user. In some cases, the sample chamber can be configured to treat and/or stabilize the sample before storing the sample. In such cases, the sample chamber can be configured to treat and/or stabilize the sample (1) while the sample of the subject is being collected into the sample chamber (e.g., from the sample acquisition device disclosed herein) and/or (2) after the sample of the subject is collected into the sample chamber. In some examples, the cartridge assembly can comprise a containment unit and a treatment/stabilization unit. The containment unit can be configured to hold the sample prior to the treatment and/or stabilization of the sample. The treatment/stabilization unit (e.g., one or more blood separation membranes, sample collection media, etc.) can be configured to treat and/or stabilize the sample that is directed or provided from the containment unit or the sample acquisition device. The sample chamber can further comprise a storage unit (e.g., a container, vessel, compartment, etc.) to store a final product of the treatment and/or stabilization of the sample by the treatment/stabilization unit. In other examples, the treatment/stabilization unit can be configured to store the final product, and the cartridge assembly may not and need not comprise a separate storage unit. In different examples, the sample chamber itself can be a storage unit. An inner surface of the sample chamber can comprise active molecules for treatment/stabilization of the sample. Alternatively, the collected sample inside the sample chamber may not and need not be treated and/or stabilized during storage.

The sample chamber disclosed herein can be modular. For example, the sample chamber can be a cartridge assembly that is modular. The cartridge assembly can comprise a plurality of modules (or subassemblies), such as a housing unit, a connection unit configured to couple to the sample acquisition device, a containment unit, a treatment/stabilization unit, a storage unit, and/or a handle (e.g., for handling of the cartridge assembly by the user). An individual unit or module of the cartridge assembly can be a replaceable or swappable unit. In some cases, after a single use of the cartridge assembly, an individual unit of the cartridge assembly can be replaced with a new unit while one or more other units of the cartridge assembly can be reusable. One or more units of the cartridge assembly can be reusable for at least 1, 2, 3, 4, 5, 6, 7, 8, 9, 10 or more uses of the cartridge assembly. In comparison to a non-modular cartridge assembly (e.g., one that cannot be easily broken down into a plurality of components), the modular cartridge assembly can comprise one or more benefits, such as ease of partial replacement, partial maintenance or repair, partial upgrade, cleaning, reduced cost of manufacturing or packaging, etc. A modular cartridge assembly can be configured for a single use only. In other embodiments, the cartridge assembly may not and need not be modular. In an example, the cartridge assembly can be configured for a single use only and may not need any partial replacement or cleaning.

The sample chamber can be configured to perform separation of one or more components from the collected sample. There can be many methods for performing blood separation, some of which use size, deformability, shape or any combination thereof. Separation can occur through one or more membranes, chambers, filters, polymers, or other materials. Membranes, substrates, filters and other components of the device can be chemically treated to selectively stabilize components, facilitate flow of sample, dry the sample, or any combination thereof. Alternative separation mechanisms can include liquid-liquid extraction, solid-liquid extraction, and selective precipitation of target or non-target elements, charge separation, binding affinity, or any combination thereof. A separation phase can comprise one or more steps, with each step relying on different mechanisms to separate the sample. One such mechanism can utilize size, shape or deformation to separate larger components from smaller ones. Cell separation can occur through a sorter that can, for example, utilize one or more filters or other size exclusion methods to separate components of the sample. Separation can also be conducted through selective binding, wherein specific components are separated by binding events while the unbound eluant moves into or through alternate chambers.

In some of the devices, systems, methods, or kits disclosed herein, a single membrane, substrate, or filter can be used for separation and collection of one or more sample components from a bulk sample. Single membrane, substrate, or filter methods can comprise a device wherein samples can be applied to one end of the membrane, substrate, or filter. As the sample flows through the membrane, substrate, or filter, a first component of the sample, for example cells, can be separated from a second component of the sample, for example plasma, based on the size of the membrane, substrate, or filter pores. After operation of the device, the membrane, substrate, or filter containing the first component of the sample, cells in this example, can be severed from the portion containing the second component of the sample, plasma in this example, necessitating an additional step of severing the membranes, substrates, or filters. In another method, two separate membranes, substrates, or filters can be used for the separation and collection sample components; for example, a first membrane, substrate, or filter for the separation of one component, for example blood cells, and a second membrane, substrate, or filter for collection of other components, for example plasma. The membranes, substrates, or filters can be arranged such that a distal end of the first membrane, substrate, or filter contacts a proximal end of the second membrane to facilitate the separation of a large component, for example cells, via the first membrane, substrate, or filter and the collection of a second smaller component, for example plasma, via the second membrane, substrate, or filter.

### 1. Blood Separation

An aspect of the present disclosure provides a sample chamber for treatment of a sample (e.g., blood) from a subject. Such treatment can comprise separation of at least a portion of the collected blood from the rest of the collected blood, as disclosed herein. In some embodiments, the sample chamber can be a cartridge assembly comprising a cartridge (e.g., at least 1, 2, 3, 4, 5, or more cartridges). In some embodiments, the cartridge assembly can be configured to separate (e.g., isolate or filter) one or more components of the blood. The blood components can comprise plasma, serum, cells (e.g., leukocytes (white blood cells) and/or erythrocytes (red blood cells)), polypeptide molecules (e.g., proteins, such as growth factors), polynucleotide molecules (e.g., DNA, RNA, cell-free DNA (cfDNA), cell-free RNA (cfRNA), etc.), ions, and/or small molecules (e.g., nutrients). In some examples, the cartridge assembly can be configured to selectively separate any number of sample components including cells, plasma, serum, platelets, specific cell types, DNA (e.g., tumor cfDNA), RNA, protein, inorganic materials, drugs, or any other components.

The cartridge assembly can comprise a cartridge port (i.e., an inlet port) that is configured to couple to a sample acquisition device. The sample acquisition device can be configured to retrieve the blood from the subject, such as any of the sample acquisition device (e.g., the device 100 as illustrated in FIG. 1) disclosed herein. The cartridge assembly can further comprise a slot (e.g., a pocket) configured to support at least one blood separation membrane. The at least one blood separation membrane can be configured to separate plasma or serum from the blood. In some embodiments, the cartridge port can comprise a pathway that is configured to direct the blood to flow from the sample acquisition device, through the pathway, and towards the at least one blood separation membrane.

In some embodiments, a direction of flow of the blood through the at least one blood separation membrane can be different from a direction of flow of the blood through the cartridge port. In some examples, the direction of flow of blood through the cartridge port can be substantially parallel to the longitudinal axis of the cartridge assembly, and the direction of flow of blood through the at least one blood separation membrane can be different than the longitudinal axis of the cartridge assembly. The direction of flow of blood through the at least one blood separation membrane may not be on the sample plane as the longitudinal axis of the cartridge assembly. The direction of flow of blood through the at least one blood separation membrane can be offset by the direction of flow of blood through the cartridge port by at least about 5 degrees, 10 degrees, 15 degrees, 20 degrees, 30 degrees, 40 degrees, 50 degrees, 60 degrees, 70 degrees, 80 degrees, 90 degrees, 100 degrees, 110 degrees, 120 degrees, 130 degrees, 140 degrees, 150 degrees, 160 degrees, 170 degrees, 175 degrees, or more. The direction of flow of blood through the at least one blood separation membrane can be offset by the direction of flow of blood through the cartridge port by at most about 170 degrees, 160 degrees, 150 degrees, 140 degrees, 130 degrees, 120 degrees, 110 degrees, 100 degrees, 90 degrees, 80 degrees, 70 degrees, 60 degrees, 50 degrees, 40 degrees, 30 degrees, 20 degrees, 10 degrees, 5 degrees, or less. In a preferred example, the direction of flow of blood through the at least one blood separation membrane can be substantially orthogonal to the direction of flow of blood through the cartridge port.

In some cases, the pathway can be configured to direct the blood to flow from the sample acquisition device into a proximal end of the pathway in a first direction, through the pathway, and exit from a distal end of the pathway and towards (e.g., onto) the at least one blood separation membrane in a second direction that is different from the first direction. In some examples, the proximal end of the pathway can be configured to receive the blood from a recessed opening in any of the sample acquisition devices disclosed herein.

The blood separation membrane can be a liquid, semi-liquid, solid, semi-solid, gel, paste, slurry, powder, gas, or a mixture thereof. The structure of the blood separation membrane can be solid, porous, symmetric, asymmetric, or a mixture thereof. A variety of membranes and fibrous elements can be suitable for use as the blood separation membrane, e.g., polymeric membranes and polymeric fibrous elements. Examples of suitable polymers can include, but are not limited to, polyolefins, polyesters, polyamides, polysulfones, acrylics, polyacrylonitriles, polyaramides, polyarylene oxides and sulfides, and polymers and copolymers made from halogenated olefins and unsaturated nitriles. For example, polyvinylidene difluoride (PVDF), polyethylene, polypropylene, polybutylene terephthalate (PBT), polyethylene terephthalate (PET), or any nylon, e.g., Nylon 6, 11, 46, 66, and 610, can be used as part of the blood separation membrane. Other suitable materials for the blood separation membrane can include cellulosic derivatives, such as cellulose acetate, cellulose propionate, cellulose acetate-propionate, cellulose acetate-butyrate, and cellulose butyrate. Non-resinous materials, such as glass fibers, including, for example, borosilicate glass fibers, can also be used.

In some cases, the cartridge assembly can comprise one or more different types of the cartridge port. Different types of the cartridge ports can be configured or customized to be compatible with different types of sample acquisition devices. Different types of cartridge ports can be configured to control or alter blood collection (e.g., velocity, volume, etc.) by the cartridge assembly.

FIG. 3A illustrates perspective views of an example cartridge assembly 300, in accordance with some embodiments. The cartridge assembly 300 can comprise a cartridge 310 that encloses a treatment/stabilization unit 320 comprising at least one blood separation membrane. In some cases, the cartridge can enclose (e.g., completely seal) the entire treatment/stabilization unit. In other examples, the cartridge can partially cover the treatment/stabilization unit. The cartridge can be directly in contact with the external surface of the treatment/stabilization unit. Alternatively, the cartridge can be separated from the external surface of the treatment/stabilization unit by a spacing or spacer (e.g., via air, gas, fluid, or other solid or semi-solid materials). A position of the treatment/stabilization unit relative to the cartridge can be fixed (e.g., immobilized). Alternatively, the position of the treatment/stabilization unit relative to the cartridge can be movable, e.g., to control flow of the blood into the treatment/stabilization unit, or to move the treatment/stabilization unit to elsewhere within the cartridge assembly prior to, during, and/or subsequent to the separation process.

FIG. 3B illustrates a side sectional view of the cartridge assembly 300 comprising the cartridge 310. The cartridge 310 can comprise the cartridge port 330, which can be configured to couple to the sample acquisition device. Various coupling mechanisms can be utilized to couple the cartridge port to the sample acquisition device. Examples of the coupling mechanisms can include, but are not limited to, male-to-female fasteners (e.g., mating or interlocking fasteners, hooks and holes, hooks and loops such as Velcro™, a female nut threaded onto a male bolt, a male protrusion inserted into a female indentation, a male threaded pipe fitted into a female threaded elbow in plumbing, a male universal serial bus (USB) plug inserted into a female USB socket, etc.), tethers (e.g., string tethers), adhesives (e.g., solids, semi-solids, gels, viscous liquids, etc.), magnets (e.g., electromagnet or permanent magnet), and other grasping mechanisms (e.g., one or more robotic arms). In an example, the coupling can be performed using an electric field between the inlet port and the sample acquisition device. In another example, the cartridge port can include a luer type fitting (e.g., as illustrated in FIG. 3B) to couple (or mate) with the sample acquisition device. The female portion of the fitting can close off a portion of the blood inlet groove to help contain the flow of blood until it nears the stack. The coupling mechanism can be reversible, such that the cartridge can be removed from the sample acquisition device once collection of the sample from the subject is completed. The coupling mechanism can be leak-free, e.g., to prevent leakage of the sample during the collection and/or separation process.

In some embodiments, as illustrated in FIGS. 3B and 3C, the cartridge port 330 of the cartridge 310 can comprise the pathway 340, which can be configured to direct the blood to flow from the sample acquisition device into a proximal end of the pathway in a first direction (as indicated by the arrow 342), through the pathway, and exit from a distal end of the pathway onto a portion (e.g., a corner, an edge, a side, or a surface) of the treatment/stabilization unit 320 in a second direction (as indicated by the arrow 344) that is different from the first direction. The pathway can comprise one or more inlet grooves (or channels). In some cases, the pathway can comprise a single groove to direct flow of the blood. In other examples, the pathway can comprise a plurality of grooves, e.g., at least 2, 3, 4, 5, 6, 7, 8, 9, 10, or more grooves. The plurality of grooves can be in fluidic communication with each other at one or more junctions. Alternatively, the plurality of grooves may not or need not be in fluidic communication with each other. The distal ends of the plurality of grooves can be directed to the same portion of the treatment/stabilization unit. Alternatively, the distal ends of the plurality of grooves can be directed to different portions of the treatment/stabilization unit, e.g., to enhance exposure of the treatment/stabilization unit to the blood. The distal ends of the plurality of grooves can allow the blood to exit in the same direction. Distal ends of the plurality of grooves can allow the blood to exit in different directions.

In some cases, an angle between the first direction (e.g., the arrow 342) and a longitudinal axis (e.g., as indicated by the arrow 346 in FIG. 3B) of the cartridge can be greater than zero degree and less than 180 degrees. The angle between the first direction and the longitudinal axis can be greater than at least 0 degree, 1 degree, 5 degrees, 10 degrees, 15 degrees, 20 degrees, 30 degrees, 40 degrees, 50 degrees, 60 degrees, 70 degrees, 80 degrees, 90 degrees, 100 degrees, 110 degrees, 120 degrees, 130 degrees, 140 degrees, 150 degrees, 160 degrees, 170 degrees, 175 degrees, or more. The angle between the first direction and the longitudinal axis can be less than at most 180 degrees, 170 degrees, 160 degrees, 150 degrees, 140 degrees, 130 degrees, 120 degrees, 110 degrees, 100 degrees, 90 degrees, 80 degrees, 70 degrees, 60 degrees, 50 degrees, 40 degrees, 30 degrees, 20 degrees, 10 degrees, 5 degrees, 1 degree, or less.

In some cases, an angle between the second direction (e.g., the arrow 344) and a longitudinal axis (e.g., the arrow 346) of the cartridge can be greater than zero degree and less than 180 degrees. The angle between the second direction and the longitudinal axis can be greater than at least 0 degree, 1 degree, 5 degrees, 10 degrees, 15 degrees, 20 degrees, 30 degrees, 40 degrees, 50 degrees, 60 degrees, 70 degrees, 80 degrees, 90 degrees, 100 degrees, 110 degrees, 120 degrees, 130 degrees, 140 degrees, 150 degrees, 160 degrees, 170 degrees, 175 degrees, or more. The angle between the second direction and the longitudinal axis can be less than at most 180 degrees, 170 degrees, 160 degrees, 150 degrees, 140 degrees, 130 degrees, 120 degrees, 110 degrees, 100 degrees, 90 degrees, 80 degrees, 70 degrees, 60 degrees, 50 degrees, 40 degrees, 30 degrees, 20 degrees, 10 degrees, 5 degrees, 1 degree, or less.

In some cases, an angle of intersection between the first direction (e.g., the arrow 342) and the second direction (e.g., the arrow 344) is greater than zero degree and less than 180 degrees. The angle of intersection between the first direction and the second direction can be greater than at least 0 degree, 1 degree, 5 degrees, 10 degrees, 15 degrees, 20 degrees, 30 degrees, 40 degrees, 50 degrees, 60 degrees, 70 degrees, 80 degrees, 90 degrees, 100 degrees, 110 degrees, 120 degrees, 130 degrees, 140 degrees, 150 degrees, 160 degrees, 170 degrees, 175 degrees, or more. The angle of intersection between the first direction and the second direction can be less than at most 180 degrees, 170 degrees, 160 degrees, 150 degrees, 140 degrees, 130 degrees, 120 degrees, 110 degrees, 100 degrees, 90 degrees, 80 degrees, 70 degrees, 60 degrees, 50 degrees, 40 degrees, 30 degrees, 20 degrees, 10 degrees, 5 degrees, 1 degree, or less.

The pathway can comprise at least one turn, such that the proximal end and the distal end are oriented in or face different directions. In some examples, the pathway can comprise one or more bent, curved, or angled portions between the proximal end and the distal end. A change of the angle within the pathway within the turn can be sudden or gradual. The pathway can comprise a plurality of turns, such that the proximal end and the distal end are oriented in the same direction.

For any subject cartridge assembly disclosed herein, the surface of the pathway (e.g., the pathway 340 as shown in FIG. 3B) can be coated with a protective agent. The protective agent can help maintain integrity or quality of the blood while it is transported to the treatment/stabilization unit. In some embodiments, the protective agent can prevent coagulation of the blood. The protective agent can comprise an anticoagulant agent, such as, but are not limited to, unfractionated heparin (“UFH”), low molecular weight heparin (“LMWH”), fondaparinux, and other antithrombin binding anticoagulants, direct factor Xa and factor IIa inhibitors, dabigatran or PRADAXA®, argatroban or ARGATROBAN®, rivaroxaban or XARELTO®, apixaban or ELIQUIS®, edoxaban or LIXIANA®, fondaparinux or ARIXTRA®, etc. In some cases, the protective agent can comprise EDTA. In some embodiments, the surface of the pathway can be free of any blood coagulation activator. For example, this can be useful when the treatment/stabilization unit is used to separate plasma from non-coagulated blood. In other examples, the treatment/stabilization unit can be configured to separate serum from coagulated blood, and in such cases, coagulation of the blood can be initiated after the blood has exited from the distal end of the pathway and towards the treatment/stabilization unit. In alternative embodiments, at least a portion of the surface of the pathway can be coated with a blood coagulation activator, such as, but are not limited to, a thrombin activator, a fibrinogen activator, metallic salt (e.g., calcium chloride, calcium gluconate), etc. In some examples, the distal end of the pathway can include the blood coagulation activator to initiate coagulation of the collected blood as the blood reaches the treatment/stabilization unit.

For any subject cartridge assembly disclosed herein, the surface of the pathway can be coated with anti-adhesive agents configured to prevent adhesion of the blood (or one or more components thereof) to the surface. In some cases, the anti-adhesive agent can be a polymer, e.g., a fluoropolymer. Examples of the fluoropolymer can include, but are not limited to, polyvinylidene fluoride (PVDF), ethylenchlorotrifluoroethylene (ECTFE), ethylenetetrafluoroethylene (ETFE), polytetrafluoroethylene (PTFE), perfluoroalkoxy (PFA), and modified fluoroalkoxy (a copolymer of tetrafluoroethylene and perfluoromethylvinylether, also known as MFA).

For any subject cartridge assembly disclosed herein, at least a portion of a surface of the sample chamber can be coated with a binding moiety configured to bind to a specific target molecule within the collected blood. For example, the binding moiety can be coupled (e.g., coated) to the treatment/stabilization unit as disclosed herein (e.g., one or more blood separation membranes, sample collection media, etc.) such that the binding moiety can come in contact with at least a portion of the collected blood. Examples of the binding moiety can include, but are not limited to, a small molecule, lipid, polypeptide (e.g., a peptide or a protein, such as an antibody, fragment thereof, or a functional variant thereof), polynucleotide (e.g., a ribonucleic acid, a deoxyribonucleic acid, a peptide nucleic acid, etc.), a cell or a fragment thereof, variations thereof, and combinations thereof. For example, the binding moiety can be an antibody or a functional variant thereof configured to bind to a specific target molecule (i.e., an antigen) in the collected blood. A non-limiting example of such antigen can include a small molecule or polypeptide (e.g., a protein or a fragment thereof). The small molecule can be a drug, e.g., to determine persistence or half-life of the drug in the subject's body. The polypeptide can be a target protein or a fragment thereof that is regulated by a drug administered to the subject, e.g., to determine efficacy of the drug therapy in regulating (e.g., upregulating, maintaining, or downregulating) expression of the target protein in the subject. The binding moiety can be useful in identifying or determining a presence of a specific cell type, disease, or condition (e.g., pregnancy, tumor, cancer, etc.) of the subject. In some cases, the binding moiety can be labeled (e.g., with a colored and/or magnetic particle (e.g., a nanoparticle or a microparticle) or a fluorophore) to allow qualitative and/or quantitative measurement of the target molecules bound by initial binding moiety. For instance, a change in the magnetic, fluorescence, and/or movement (e.g., vibration) of such label can be measured as an indication of the target molecules bound by initial binding moiety. An additional binding moiety that is different from the initial binding moiety (that is coupled to a portion of the sample chamber) can be applied for analyzing of the amount of target molecules that are bound by the initial binding moiety. In some examples, similar to a Sandwich Enzyme-Linked ImmunoSorbent Assay (ELISA), the additional binding moiety can be an antibody that binds to a different region of the target molecule than the initial binding moiety. The additional binding moiety can be labeled (e.g., with a colored and/or magnetic particle (e.g., a nanoparticle or a microparticle) or a fluorophore) to qualitative and/or quantitative measurement of the target molecules bound by the initial binding moiety.

The term “antibody,” as used herein, refers to a proteinaceous binding molecule with immunoglobulin-like functions. The term antibody includes antibodies (e.g., monoclonal and polyclonal antibodies), as well as derivatives, variants, and fragments thereof. Antibodies can include immunoglobulins (Ig's) of different classes (i.e. IgA, IgG, IgM, IgD an d IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)2, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies). In some cases, the antibody can be a humanized antibody.

Examples of cells that can be identified by the binding moiety can include, but are not limited to, lymphoid cells, such as B cell, T cell (Cytotoxic T cell, Natural Killer T cell, Regulatory T cell, Helper T cell), Natural killer cell, cytokineCytokine-induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffin cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Leydig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous systemneurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, Interstitial kidney cells, and fetal cells (e.g., fetal blood cells, such as fetal nucleated red blood cells for detection of fetal abnormalities during pregnancy).

Additional examples of cells that can be identified by the binding moiety can be include, but are not limited to, cancer or tumor cells, such as those from cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms' tumor.

For any subject cartridge assembly disclosed herein, the slot (e.g., the slot 350 of the cartridge 310, as shown in FIG. 3B) can be configured to support the treatment/stabilization unit. The treatment/stabilization unit can be supported and held in space within the slot with the aid of an adhesive. The adhesive can be a hydrogel, an acrylic, a polyurethane gel, a hydrocolloid, or a silicone gel. Alternatively, the treatment/stabilization unit can be supported and held in space within the slot without the aid of an adhesive.

The at least one blood separation membrane 322 can be part of the treatment/stabilization unit 320, as illustrated in FIG. 3B. The cartridge assembly can comprise at least 1, 2, 3, 4, 5, 6, 7, 8, 9, 10, or more blood separation membranes. In some examples, the cartridge assembly can comprise a plurality of blood separation membranes. The plurality of blood separation membranes can be in fluidic communication with each other, e.g., to allow the blood sample to be subjected to multiple separation processes. The plurality of blood separation membranes can be provided in series. Alternatively, the plurality of blood separation membranes may not and need not be in fluidic communication with each other, e.g., each blood separation membrane can be configured to separate different portions of the collected blood. In some embodiments, the plurality of blood separation membranes can be provided in parallel.

The slot can be further configured to support a collection media (or a collection agent) for collecting a product of the blood separation (e.g., separated plasma or serum) by the blood separation membrane. The collection media can be paper, for example a cellulose paper. The collection media can be a fiber material, for example a cellulose fiber material. The collection media can comprise, for example, one or more materials selected from the group consisting of: polyester, polyether sulfone (PES), polyamide (Nylon), polypropylene, polytetrafluoroethylene (PTFE), polycarbonate, cellulose nitrate, cellulose acetate, and aluminum oxide. The slot can comprise at least 1, 2, 3, 4, 5, 6, 7, 8, 9, 10, or more collection media (e.g., one or more cellulose paper sheets). As illustrated in FIG. 3B, the collection media 324 can be disposed adjacent to the blood separation membrane 322. In some cases, the collection media and the blood separation membrane can be disposed directly adjacent to each other without any gap (e.g., a gap of air) therebetween. Alternatively, the collection media and the blood separation membrane can be disposed adjacent to each other with a spacing therebetween, e.g., to provide time for the collection media to absorb the product of the blood separation process from the blood separation membrane. The collection media can comprise at least 1, 2, 3, 4, 5, 6, 7, 8, 9, 10, or more sheets of the paper disclosed herein.

The collection media can have a volume sufficient to collect a desired amount of the product (e.g., serum or plasm) of the blood separation membrane. The collection media can be configured to hold (or contain) at least about 1 μL, 5 μL, 10 μL, 20 μL, 30 μL, 40 μL, 50 μL, 60 μL, 70 μL, 80 μL, 90 μL, 100 μL, 110 μL, 120 μL, 130 μL, 140 μL, 150 μL, 200 μL, 300 μL, 400 μL, 500 μL, 600 μL, 700 μL, 800 μL, 900 μL, 1,000 μL, or more of the product of the blood separation membrane. The collection media can be configured to hold (or contain) at most about 1,000 μL, 900 μL, 800 μL, 700 μL, 600 μL, 500 μL, 400 μL, 300 μL, 200 μL, 100 μL, 50 μL, 10 μL, 1 μL, or less of the product of the blood separation membrane.

The slot can be configured to support a pre-filter. The pre-filter can be configured for filtering the blood prior to separating the plasma or serum from the blood. The pre-filter can help increase the amount and/or speed of blood separation per area of the at least one blood separation membrane (e.g., by at least 1-fold, 2-fold, 3-fold, 4-fold, 5-fold, 6-fold, 7-fold, 8-fold, 9-fold, 10-fold, 15-fold, 20-fold, 30-fold, or more in comparison to at least one blood separation membrane that is not operatively coupled to a pre-filter). The pre-filter can be a filter paper, such as a glass fiber paper or a cellulose filter paper. Any suitable commercially available filter paper can be used. Examples of commercially available filter paper include, but are not limited to, filter paper from Whatman®, such as fast transit analysis (FTA®) card. In some embodiments, the pre-filter can comprise a nitrocellulose filter paper. The pre-filter can comprise at least 1, 2, 3, 4, 5, 6, 7, 8, 9, 10, or more sheets of the filter paper disclosed herein. As illustrated in FIGS. 3B and 3C, the pre-filter 326 can be disposed adjacent to the blood separation membrane 322. The pre-filter 326 and the collection media 324 can be disposed on different portions (e.g., opposite sides or surfaces) of the separation membrane 322. In some cases, the blood separation membrane and the pre-filter can be disposed directly adjacent to each other without any gap (e.g., an airgap) therebetween. Alternatively, the blood separation membrane and the pre-filter can be disposed adjacent to each other with a spacing therebetween, e.g., to provide time for the blood separation membrane to absorb and/or filter the blood. As illustrated in FIGS. 3B and 3C, the distal end of the pathway 340 can be positioned or oriented such that the blood is transported from the sample acquisition device and towards (e.g., directly towards) the pre-filter 326. In some examples, the blood separation membrane of the cartridge assembly can be configured to separate plasma or serum from the collected blood, and thus the pre-filter can filter (e.g., retain) any number of other non-desirable sample components including cells, platelets, specific cell types, DNA (e.g., tumor cfDNA), RNA, protein, inorganic materials, drugs, or any other components.

In some embodiments, the cartridge assembly may not and need not have a pre-filter in the slot. For example, the distal end of the pathway of the cartridge can be disposed such that the blood is transported from the sample acquisition device and towards (e.g., directly towards) the blood separation membrane.

As illustrated in FIG. 3B, the blood separation membrane 322, the collection media 324, and the pre-filter 326 can be collectively provided as a treatment/stabilization unit 320 within the slot 350. The treatment/stabilization unit can be interchangeably referred herein as a stack. The cartridge can comprise a single treatment/stabilization unit. Alternatively, the cartridge can comprise a plurality of treatment/stabilization units (e.g., a plurality of treatment/stabilization units disposed in parallel or in series). In some examples, a plurality of treatment/stabilization units can be disposed on the same plane within the cartridge. In other examples, a plurality of treatment/stabilization units can be disposed on different planes within the cartridge. The cartridge can comprise at least 1, 2, 3, 4, 5, 6, 7, 8, 9, 10, or more stacks. The cartridge can comprise at most 10, 9, 8, 7, 6, 5, 4, 3, 2, or 1 stack.

In some embodiments, the stack (e.g., treatment/stabilization unit 320) can be disposed in a configuration that permits lateral flow of the blood through a thickness of the stack in a third direction (e.g., that is different from the longitudinal axis 346), and/or planar flow across a planar area of the stack in at least one other direction (e.g., the same planar direction as the longitudinal axis 346) that is different from the third direction. In some cases, the third direction can be different from the first direction and/or the second direction. An angle between the third direction (for the lateral flow of the blood or one or more components thereof) and the longitudinal axis can be greater than at least 0 degree, 1 degree, 5 degrees, 10 degrees, 15 degrees, 20 degrees, 30 degrees, 40 degrees, 50 degrees, 60 degrees, 70 degrees, 80 degrees, or more. The angle between the third direction (for the lateral flow of the blood) and the longitudinal axis can be less than at most 90 degrees, 80 degrees, 70 degrees, 60 degrees, 50 degrees, 40 degrees, 30 degrees, 20 degrees, 10 degrees, 5 degrees, 1 degree, or less. The at least one other direction (for the planar flow of the blood or one or more components thereof) can be the same as the longitudinal axis 346. Alternatively, an angle between the at least one other direction and the longitudinal axis 346 (for the planar flow of the blood or one or more components thereof) can be greater than at least 0 degree, 1 degree, 5 degrees, 10 degrees, 15 degrees, 20 degrees, 30 degrees, 40 degrees, 50 degrees, 60 degrees, 70 degrees, 80 degrees, or more. The angle between the at least one other direction and the longitudinal axis 346 (for the planar flow of the blood or one or more components thereof) can be at most 90 degrees, 80 degrees, 70 degrees, 60 degrees, 50 degrees, 40 degrees, 30 degrees, 20 degrees, 10 degrees, 5 degrees, 1 degree, or less. As shown in FIG. 3C, the blood 370 is transported from the sample acquisition device, through the pathway, and towards the stack. The blood can be directed to the planar surface of the stack (e.g., the planar surface of the pre-filter). The third direction (i.e., the direction through a thickness of the stack) can be substantially orthogonal to the longitudinal axis 346 of the cartridge. In addition, the third direction e., the direction through a thickness of the stack) and the at least one other direction (i.e., the direction across a planar area of the stack) can be substantially orthogonal to one another.

Each layer of the stack (e.g., the pre-filter, the blood separation membrane, and/or the collection media) can have various shapes and sizes. For example, a layer can be in the shape of a rectangle, sphere, cuboid, or disc, or any partial shape or combination of shapes thereof. The layer can have a cross-section that is circular, elliptical, oval, triangular, square, rectangular, pentagonal, hexagonal, or any partial shape or combination of shapes thereof. In some embodiments, each layer of the stack can have the same shape, thickness, length, width, depth, volume, or surface area. In other embodiments, each layer of the stack may not and need not have the same shape or dimension. In some cases, the layers of the stack can have different shapes and sizes to achieve different separation throughputs, overall yields, and collected volumes. In an example, the width of the collection media (i.e., a collection strip) can be longer than other layers, so as to create a “tail” end to help separate from the other layers. Such separation can help to reduce loss of the collected product (e.g., serum or plasma) from the collection media.

In some embodiments, the distal end of the pathway can be offset from a linear axis that extends between (1) the proximal end of the pathway and (2) an edge thickness portion of the stack. As illustrated in FIG. 3B, the edge thickness portion (where the bracket sign “{” is located in FIG. 3B) of the stack 320 can be located between the proximal end and the distal end of the pathway. The distal end of the pathway can be adjacent to, but need not be in contact with the planar surface of the pre-filter. In some cases, the blood can exit from the distal end of the pathway into a spacing or a void 360. Subsequently, the blood from the spacing 360 can be directed or drawn towards the stack (e.g., an exposed surface of the pre-filter 326 or an exposed surface of the blood separation membrane 322). In some examples, the spacing 360 can be in fluidic communication with an accumulation region 362 disposed adjacent to the distal end of the pathway and the stack 320. The accumulation region can comprise a separate blood containment container or cup that is configured to hold a volume of the blood. The blood containment cup can be configured to contain the blood as it is being absorbed into a portion of the blood separation membrane. In some cases, the blood containment cup can be configured to hold a predefined volume of the blood that is to be treated (e.g., separated) by the stack 320. The shape of the cup (or pocket) can be optimized to direct varying volumes of the blood to different portions of the stack surface (e.g., to contain most of the input blood near the surface of the stack adjacent to the distal end of the pathway, or to assist the incoming blood to spread along the planar surface of the stack). The shape of the cup can be optimized to adjust the concentration of the blood across the pre-filter, or to adjust the volume of the blood that is contained. For example, the cup can be in the shape of a sphere, cuboid, or disc, or any partial shape or combination of shapes thereof. The cup can have a cross-section that is circular, elliptical, oval, triangular, square, rectangular, pentagonal, hexagonal, or any partial shape or combination of shapes thereof. The cup can be configured to hold a predetermined volume of the collected blood. The cup can be configured to hold at least about 1 μL, 5 μL, 10 μL, 20 μL, 30 μL, 40 μL, 50 μL, 60 μL, 70 μL, 80 μL, 90 μL, 100 μL, 110 μL, 120 μL, 130 μL, 140 μL, 150 μL, 200 μL, 300 μL, 400 μL, 500 μL, 600 μL, 700 μL, 800 μL, 900 μL, 1,000 μL, or more of the blood. The cup can be configured to hold at most about 1,000 μL, 900 μL, 800 μL, 700 μL, 600 μL, 500 μL, 400 μL, 300 μL, 200 μL, 100 μL, 50 μL, 10 μL, 1 μL, or less of the blood. In some examples, the cup can be used as a metering device (e.g., a metering window) to determine when (or whether) sufficient blood has been collected into the cartridge assembly.

In some embodiments, the distal end of the pathway can be adjacent to or directly in contact with the planar surface of the pre-filter.

The pathway of the cartridge port (i.e., the inlet port) of the cartridge assembly can comprise an opening (or a cut-out) that exposes a portion of the pathway along a length of the cartridge port. The pathway can be in fluidic (e.g., gaseous or liquid) communication with an inner portion (e.g., the recess) of the sample acquisition device disclosed herein, via an opening. The opening can be sealed prior to use of the cartridge assembly. In some cases, the opening can be partially or completely exposed to the inner portion of the sample acquisition device upon a complete installation (e.g., insertion) of the cartridge assembly into the device.

In some embodiments, the cartridge assembly can be subject to vacuum pressure when a vacuum in the sample acquisition device is activated (e.g., manually by the user or automatically by operation of the sample acquisition device on the user). The vacuum pressure can be configured to assist with lateral flow of the blood through and/or across the stack on the cartridge. In alternative embodiments, the cartridge assembly can be under vacuum pressure prior to its installation into the sample acquisition device. In some examples, while the vacuum pressure can vent into the sample acquisition device upon a complete installation of the cartridge assembly into the sample acquisition device, sufficient negative pressure can remain within the cartridge assembly to assist with lateral flow of the blood through and/or across the stack within the cartridge.

FIG. 3D shows a side sectional view of a sample acquisition device 100 operatively coupled to the cartridge assembly 300, in accordance with some embodiments. As illustrated, the blood can be transported from an opening in the recess 136 of the device 100, through the pathway of the cartridge, and towards the stack (comprising at least one blood separation membrane). The cartridge assembly 300 can comprise a cartridge holder/tab 380 configured to seal the cartridge inside the device, e.g., within the deposition/separation chamber. The cartridge assembly can comprise at least 1, 2, 3, 4, 5, 6, 7, 8, 9, 10, or more absorption pads. Depending on the location of the absorption pad relative to the blood separation membrane (or the stack), the absorption pad can be used to absorb excess blood and/or excess serum or plasma. In some cases, one or more absorption pads can be disposed directly adjacent to the blood separation membrane (or the stack). Alternatively, or in addition to the above embodiments, one or more absorption pads can be in fluidic communication with, but physically isolated from the blood separation membrane (or the stack). Isolation of the one or more absorption pads can reduce the risk of (1) contamination of, or (2) excessive absorption or wicking of the blood (or serum/plasma) from the blood separation membrane (or the stack).

One or more components of the cartridge assembly can be configured to be released and decoupled. In some embodiments, the collection media can be configured to be released and decoupled from the cartridge (and the sample acquisition device) after the blood has been treated, e.g., after at least a portion of the plasma or serum has been separated by the blood separation membrane 322 and collected onto the collection media 324. In some examples, the remaining components of the cartridge assembly can be configured to remain coupled to the sample acquisition device after the collection media has been released and decoupled from the cartridge. In other examples, release of the collection media from the cartridge can be configured to trigger release of the cartridge from the sample acquisition device. In other examples, the cartridge assembly can be configured to be released from the sample acquisition device prior to the release of the collection media from the cartridge. Afterwards, the released collection media can be stored in a separate transport housing (e.g., the transport sleeve 200 in FIG. 2A) for transportation. In alternative embodiments, the cartridge assembly can be configured to be released from the sample acquisition device, but the collection media may not and need not be configured to be released from the cartridge. In some examples, the cartridge assembly (that comprises the collection media) as whole can be used as a transportation medium, and/or the cartridge assembly can be stored in a separate transport housing (e.g., the transport sleeve 200 in FIG. 2A) for transportation.

In some embodiments, at least a portion of the cartridge 310 of the cartridge assembly 300 can comprise a transparent or semi-transparent window. The window can be configured to permit a user to observe a progress of (1) the flow of the blood within the pathway 340 of the inlet port, (2) the flow of the blood from the distal end of the pathway 340 and towards the cup 362 or an exposed surface of the stack 320 (e.g., an exposed surface of the pre-filter 326), and/or (3) the flow of the blood within the stack, e.g., the blood separation by the blood separation membrane 322 and towards the collection media. In some cases, the window can be located adjacent to the at least one blood separation membrane, the collection media, and/or the pre-filter. The window of the cartridge can be aligned with a viewing window or open structure of the device (e.g., the device 100). In some cases, depending on the orientation of the cartridge assembly relative to the device, either (1) the blood input into the blood separation membrane (or the pre-filter of the stack) can be viewed, or (2) the plasma or serum output from the blood separation membrane (or the stack) can be viewed. In an example, when the pre-filter facing side of the cartridge assembly faces towards the skin of the subject, the user can visualize the plasma or serum output from the blood separation membrane. In another example, when the collection media facing side of the cartridge assembly faces towards the skin of the subject, the user can visualize the blood input into the blood separation membrane (or the pre-filter of the stack).

FIGS. 3E and 3F schematically illustrate side cross-sectional views of another example of the sample chamber. The sample chamber can be the cartridge assembly 300b. The cartridge assembly can comprise one or more components of the cartridge assembly 300 as disclosed herein (e.g., in FIGS. 3B and 3C). Referring to FIG. 3E, the cartridge assembly 300b can comprise the cartridge port 330 that is coupled to the cartridge 310. The cartridge port 330 can be configured to couple (e.g., releasably couple) to the sample acquisition device using any of the coupling mechanisms described herein. The cartridge can comprise a slot 350 that encloses the treatment/stabilization unit 320. The treatment/stabilization unit 320 can comprise at least one blood separation membrane 322. In some cases, the treatment/stabilization unit 320 can further comprise the collection media 324 and/or the pre-filter 326. The cartridge port 330 can comprise a pathway 340 configured to direct the subject's sample (e.g., blood) from the sample acquisition device and towards the cartridge 310. The cartridge can further comprise a spacing 360 that is in fluid communication with the pathway 340. The spacing 360 can also be in fluid communication with the accumulation region 362 (e.g., a container or a cup) configured to hold a volume of the collected sample. The accumulation region 362 can be disposed adjacent to the treatment/stabilization unit 320, such that the collected sample can be contained within the cartridge 310 while at least a portion of the collected sample is treated/stabilized by flowing across the treatment/stabilization unit 320. As shown in FIG. 3F, the direction of the sample flow through the pathway 340 can be substantially the same as the longitudinal axis of the cartridge assembly (as indicated by the arrow 346). The direction of the sample flow through the pathway 340 can be different than the direction of flow of blood through the treatment/stabilization unit 320. In an example, the direction of the sample flow through the pathway 340 can be substantially orthogonal to the direction of flow of blood through the treatment/stabilization unit 320.

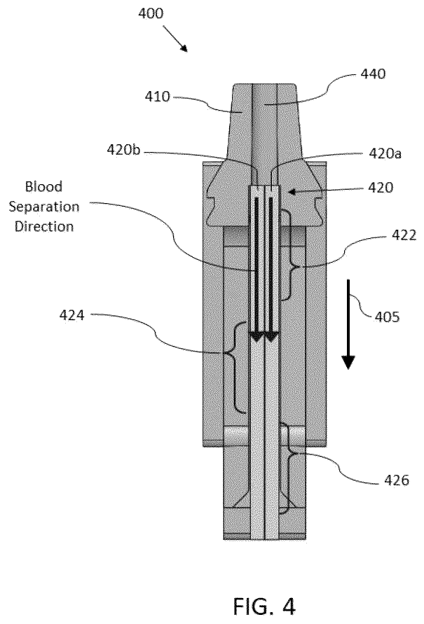

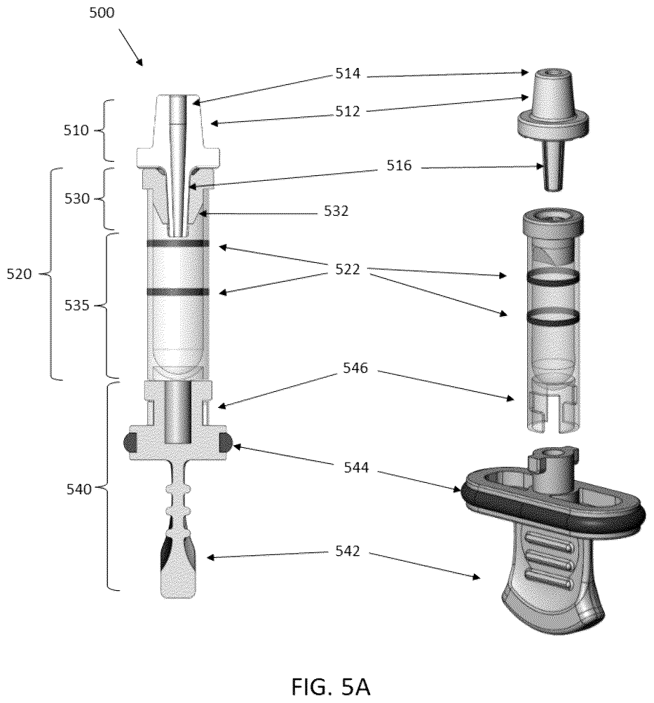

FIG. 4 shows a side section view of a different example of the sample chamber. The sample chamber can be the cartridge assembly 400. The cartridge assembly 400 can comprise one or more components of the cartridge assembly 300 disclosed herein (e.g., in FIGS. 3B and 3C). The cartridge assembly 400 can comprise a cartridge port 410 that provides a pathway 440 for the blood to be transported from the sample acquisition device (e.g., the sample acquisition device disclosed herein) and towards the treatment/stabilization unit 420 (i.e., a stack). The cartridge port 410 can be configured to couple (e.g., releasably couple) to the sample acquisition device using any of the coupling mechanisms described herein. For example, the cartridge port 410 can have a luer type fitting to mate with the sample acquisition device. The treatment/stabilization unit 420 can comprise one or more treatment/stabilization components. For example, the treatment/stabilization unit 420 can comprise a first treatment/stabilization component 420a and a second treatment/stabilization component 420b. The two treatment/stabilization components can be disposed adjacent to each other. In an example, as shown in FIG. 4, the two treatment/stabilization components can be in direct contact with each other. Alternatively, the treatment/stabilization components can be separated by a space or gap (not shown). As shown in FIGS. 3B-3D, the cartridge assembly 300 can be configured to receive the collected blood on a planar surface of the stack to allow separation of the blood to occur in a direction that is different (e.g., substantially orthogonal) from the longitudinal axis 346 of the cartridge 300 (as illustrated in FIGS. 3B-3F). In the example of FIG. 4, the cartridge assembly 400 can be configured to receive the collected blood on an edge of the treatment/stabilization unit 420 and/or a portion on a planar surface near the edge to allow separation of the blood to occur in a direction that is (i) substantially the same as the longitudinal axis 405 of the cartridge assembly 400 (as illustrated in FIG. 4) and/or (ii) on the same plane as the longitudinal axis of the cartridge assembly 400. In this case the upper portion of the treatment/stabilization component(s) will contain a filtered portion of the sample (e.g. blood cells) while the lower portion of the treatment/stabilization component(s) will contain another portion of the sample (e.g. plasma or serum). In some embodiments, each of the treatment/stabilization components(s) can comprise a plurality of components, e.g., the pre-filter, the blood separation membrane, and/or the collection media, as disclosed herein. In some examples, the top portion 422 of the treatment/stabilization unit 420 that is adjacent to the pathway 440 can comprise the pre-filter 422 that can be configured to, e.g., filter out cells from the collected blood. The middle portion 424 of the treatment/stabilization unit 420 can comprise one or more blood separation membranes. The bottom portion 426 of the treatment/stabilization unit 420 that is away from the pathway 440 can comprise the collection media. In some examples, a single treatment/stabilization component can be used (e.g., only one of 420a or 420b). Alternatively, more than two treatment/stabilization components (e.g., at least 2, 3, 4, 5, 6, 7, 8, 9, 10, or more treatment/stabilization components) can be used having all, some, or none in direct contact with one another. In some examples, a pre-filter component can reside next to the upper portion of the one or more of the treatment/stabilization components to receive the blood initially and filter out at least a portion of the sample (e.g., cells, debris, etc.) prior to allowing the rest of the sample to flow towards and arrive at the surface of the treatment/stabilization component(s). The features of the cartridge assembly 400, as shown in FIG. 4, can be applied to any device, system, method, or kit for sample collection, as disclosed herein.

Another aspect of the present disclosure provides a system for blood collection and blood separation. The system can comprise the sample acquisition device (e.g., the sample acquisition device) and the sample chamber (e.g., the cartridge assembly) as disclosed herein. In some embodiments, the sample acquisition device can comprise an onboard vacuum. Such vacuum can be sufficient to pull the subject's skin towards the sample acquisition device, to draw blood from the subject once the skin has been pierced. In alternative embodiments, the chamber of the sample acquisition device that contains the cartridge assembly can be pre-packaged with onboard vacuum, and the venting of such vacuum into the sample acquisition device can be sufficient to pull the subject's skin towards the sample acquisition device, to draw blood from the subject once the skin has been pierced.

Another aspect of the present disclosure provides a method (e.g., for blood collection and blood separation). The method can comprise using the sample acquisition device as disclosed herein to collect the blood from the subject. The method can further comprise using the sample chamber (e.g., the cartridge assembly) as disclosed herein to separate the plasma or serum from the blood. In some embodiments, the method can further comprise storing the separated plasma or serum from the blood (e.g., in the collection media 324 of the cartridge assembly 300, as shown in FIG. 3B).

### 2. Liquid Blood Collection

Another aspect of the present disclosure provides a sample chamber, such as a cartridge assembly, for storing liquid or liquid-like sample (e.g., liquid blood) that is collected from a subject via a sample acquisition device, e.g., any of the sample acquisition device as disclosed herein. In some embodiments, the cartridge assembly can comprise a coupling unit configured to couple to a portion of the sample acquisition device, e.g., a cartridge chamber. The coupling unit can comprise an inlet port. The cartridge assembly can further comprise a container configured to store the liquid or liquid-like sample. The cartridge assembly can further comprise a cartridge holder configured to support the container. A proximal end of the container can be configured to couple to the coupling unit, and a distal end of the container can be configured to couple to the cartridge holder. The liquid or liquid-like sample can be one or more members selected from the group consisting of liquid blood, sweat, tears, urine, saliva, feces, vaginal secretions, semen, interstitial fluid, mucus, sebum, crevicular fluid, aqueous humour, vitreous humour, bile, breast milk, cerebrospinal fluid, cerumen, enolymph, perilymph, gastric juice, peritoneal fluid, vomit, and the like. In an example, the liquid sample can be liquid blood.

In some embodiments, the proximal end of the container of the cartridge assembly can be configured to releasably couple to the coupling unit using any of the coupling mechanisms described herein. In some cases, the container can comprise a container port that is configured to releasably couple to the coupling unit. The container port can be a part of the container. The container port can be releasably coupled to the proximal end of the container. In other embodiments, the proximal end of the container may not or need not be configured to releasable coupled to the coupling unit. For example, the proximal end of the container can be permanently coupled to the coupling unit. In some embodiments, the distal end of the container can be configured to releasably couple to the cartridge holder using any of the coupling mechanisms described herein. In other embodiments, the distal end of the container may not or need not be configured to releasably couple to the cartridge holder. For example, the distal end of the container can be permanently coupled to the cartridge holder. Alternatively, the cartridge holder can be fabricated as a part of the container, e.g., as part of the distal end of the container. The proximal end of the container can comprise one or more openings configured to receive the liquid sample (e.g., the liquid blood). The distal end of the container may not comprise any opening and can be closed, to allow sample collection within at least a portion of the container.

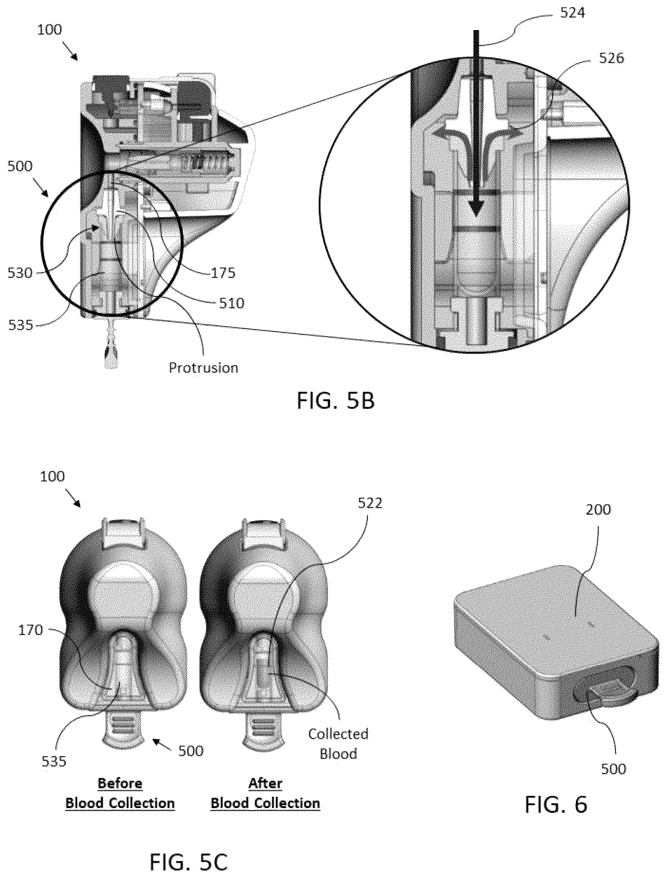

FIG. 5A illustrates a side sectional view (left side of FIG. 5A) and a perspective view (right side of FIG. 5A) of an example cartridge assembly 500 that can be configured to collect liquid or liquid-like samples (e.g., liquid blood). The cartridge assembly 500 can comprise the coupling unit 510 (interchangeably referred to herein as an adapter, or a tube inlet adapter). The coupling unit can be configured to couple (e.g., releasably or permanently couple) to the sample acquisition device (e.g., a port in a cartridge chamber of any of the sample acquisition devices disclosed herein) using any of the coupling mechanisms described herein. For example, the coupling unit 510 can have a luer type fitting 512 to mate with cartridge chamber port of the sample acquisition device. The coupling unit 510 can comprise an opening, an inlet, or a channel that is configured to serve as a pathway 514 for the blood to flow from the sample acquisition device and towards the cartridge assembly (e.g., into the cartridge assembly). The cartridge assembly 500 can comprise the container 520 that is coupled to the coupling unit 510 and the cartridge holder 540. The container can have a cross-section that is circular, elliptical, oval, triangular, square, rectangular, pentagonal, hexagonal, or any partial shape or combination of shapes thereof. The container can comprise a container port (e.g. a cap or a tube cap) 530. The container can comprise a collection tube 535 configured to contain the collected blood. The container port can be coupled to the proximal end of the container. For example, the container port (e.g., a tube cap) and the proximal end of the collection tube can be releasably coupled using any of the coupling mechanisms described herein (e.g., luer type, screw type, friction fit, etc.). In another example, the container port 530 and the collection tube 535 can be permanently coupled (e.g., glued) to each other. The proximal end of the collection tube can be configured to couple to the coupling unit via the container port. For example, the coupling unit and the container port can be releasably coupled using any of the coupling mechanisms described herein (e.g., luer type, screw type, friction fit, etc.). In another example, the coupling unit and the container port can be permanently coupled (e.g., glued) to each other. In some cases the coupling unit and the port in the cartridge chamber of the sample acquisition device can be permanently coupled. In some cases, the container port 530 and the collection tube 535 can require an alignment (e.g., a rotational alignment) to insert the cartridge assembly 500 into the sample acquisition device in a preferred orientation. The container port 530 and the collection tube 535 can utilize any of the coupling mechanisms described herein to interlock the two components when the components are aligned. In other embodiments, the container 520 may not and need not require the container port 530 to couple to the coupling unit 510. In an example, the collection tube 535 can be directly coupled (e.g., releasably coupled or permanently coupled) to the coupling unit 510.

In some embodiments, at least a portion of the collection tube can comprise a transparent or semi-transparent window. The window can be configured to permit a user to observe the blood flowing into the collection tube. In other embodiments, the collection tube itself can be transparent or semi-transparent, e.g., the collection tube can comprise of one or more transparent or semi-transparent materials. In some embodiments, a bottom of the collection tube 535 can be configured to allow the container 520 to stand, e.g., on a flat surface. For example, at least a portion of the bottom of the collection tube 535 can be flat.

In some embodiments, the container port of the container can comprise one or more openings that are configured to open and permit fluidic (e.g., gas such as air, liquid such as liquid blood, etc.) access to the container when the container port is coupled to the coupling unit. The opening(s) of the container port can have a cross-section that is circular, elliptical, oval, triangular, square, rectangular, pentagonal, hexagonal, or any partial shape or combination of shapes thereof. In some cases, the opening can utilize a fluidic regulator to control passage of the fluid into the container (e.g., from the sample acquisition device and into the container) or from within the container (e.g., from within the container and into the sample acquisition device). The fluidic regulator can comprise a mechanical regulator (e.g., a spring regulator or a self-closing flap), hydraulic regular, pneumatic regulator, manual regulator, solenoid regulator, or a motorized regulator. Examples of a fluidic regulator can include, but are not limited to, a seal, flap, valve, gate, switch, lever, pump, etc. In an example, as illustrated in FIG. 5A, the container port 530 of the container 520 can comprise an integrated self-closing valve 532 (e.g., a duckbill valve) configured to be (i) opened to permit fluidic access to the container 520 when the container port 530 is coupled to the coupling unit 510 and (ii) closed to reduce (e.g., inhibit or prohibit) fluidic access to the container 520 when the container port 530 is not coupled to the coupling unit 510. In other examples, the opening can permanently allow fluidic passage (e.g., a one-way fluidic passage in a direction from outside the cartridge assembly 500 and into the cartridge assembly 500) without the need for a fluidic regulator.

In some embodiments, the coupling unit can comprise one or more fluidic pathways (as indicated by 516 in FIG. 5A) that permit air to expunge out of the container as the blood is being collected into the container (e.g., from the sample acquisition device). In some cases, the coupling unit can connect a blood port of the sample acquisition device to the container (e.g., via the container port) of the cartridge. As a result, at least a portion of the cartridge can be located inside the sample acquisition device (e.g., within the cartridge chamber of the sample acquisition device). The cartridge chamber can be under vacuum (e.g., below ambient pressure by activation of the vacuum chamber) when the cartridge is coupled to the sample acquisition device. Subsequently, upon coupling, the air can expunge out of the container, through the one or more fluidic pathways, and into the cartridge chamber. The one or more fluidic pathways can allow pressure (e.g., vacuum pressure) within the cartridge chamber to be equalized as the blood is being collected into the container. The one or more fluidic pathways can allow the container to be evacuated, e.g., to the same pressure level as that of the surrounding cartridge chamber (e.g., still below ambient pressure). The resulting vacuum in the container can draw blood from the pierced skin of the subject, through the inlet port, and into the container of the cartridge. In some examples, air from within the container (e.g., from within the collection tube) can continue to expunge out through the fluidic pathway(s) disclosed herein as the blood is being drawn into the container. In other examples, air from within the container can expunge out through one or more semi-permeable membranes that are integrated into the collection tube of the container. The semi-permeable membrane(s) can be configured to allow air to flow while preventing liquid from flowing (e.g., exiting from within the collection tube).

In some embodiments, the blood can be drawn into the container until a desired volume of the blood is collected. In some cases, vacuum of the sample acquisition device can be configured such that it is sufficient to draw approximately the desired volume of the blood into the container. In some cases, as illustrated in FIG. 5A, the container 520 can comprise one or more indicators 522 (e.g., markings, drawings, digital indicators, etc.) that indicate to a user an approximate amount of the blood that is drawn into the container 520. The container can comprise at least 1, 2, 3, 4, 5, 6, 7, 8, 9, 10, or more indicators. The user can subsequently halt the blood drawing process (e.g., by pressing a button located on the container or on the sample acquisition device). In alternative embodiments, the indicator can comprise a sensor configured to detect or measure the presence and/or amount (e.g., weight, volume) of the collected blood in the container. In an example, when the sensor measures and determines that a desired volume of blood has been collected, the device or the cartridge can be configured to automatically halt the blood drawing process. The indicator can comprise at least 1, 2, 3, 4, 5, 6, 7, 8, 9, 10, or more sensors. Examples of the sensor can include, but are not limited to, a mechanical sensor (e.g., a scale), an optical sensor (e.g., a camera), an ultrasonic sensor (e.g., a non-contact ultrasonic level sensor), a radar sensor (e.g., a radar level transmitter), a capacitance sensor (e.g., a capacitance measurement probe), a chemical sensor, a pressure sensor, a fluid flow sensor, a humidity sensor, a vibration sensor, a field sensor (e.g., an electromagnetic sensor), a temperature sensor, etc. The sensor can be configured to come in contact with the collected blood. Alternatively, the sensor may not and need not come in contact with the collected blood for its function. In some cases, the outer surface of the tube 535 can be covered (e.g., partially or entirely masked) to allow the sensor to focus on a desired region of the tube for blood sensing.

In some cases, the indicator (e.g., the sensor) can be operatively coupled to an alert system configured to alert the user when a desired amount of the blood is collected into the tube 535. In some example, the alert system can be configured to generate an audible, tactile, and/or visual alert to the user (e.g., via a speaker or a light emitting diode (LED). The alert system can be operatively coupled to the indicator via one or more wired (e.g., digital circuits) or wireless communication channels. Examples of wireless communication channels can include Bluetooth®, WiFi, Near Field Communication (NFC), 3G, 4G, and/or 5G networks. Signals for activating the alert system can be transmitted remotely from the indicator (e.g., a sensor of the indicator) over the one or more communication channels to the alert system. In some cases, the sensor (e.g., a computer processor operatively coupled to the sensor) can be configured (or programmed) to prevent false triggering of the alert system by, for example, (1) droplets of the blood passing by the sensor and into the tube 535 or (2) wetting of an inner surface of the tube 535 by the blood being collected. In an example, sensitivity and/or threshold of the sensor can be adjusted to prevent false triggering of the alert system.

In some cases, the one or more sensors can be used to determine a presence and/or concentration of the one or more target analytes (e.g., cells, plasma, serum, platelets, specific cell types, DNA (e.g., tumor cfDNA), RNA, protein, inorganic materials, drugs, or any other components) in the fluid sample (e.g., liquid blood). For example, a sensor can determine the presence and/or presence of a target analyte in the liquid blood in the container based on detected changes to electron and ion mobility and charge accumulation when the liquid blood is collected into the container and comes in contact with the sensor.

The container can be configured to hold at least about 1 μL, 5 μL, 10 μL, 20 μL, 30 μL, 40 μL, 50 μL, 60 μL, 70 μL, 80 μL, 90 μL, 100 μL, 110 μL, 120 μL, 130 μL, 140 μL, 150 μL, 200 μL, 300 μL, 400 μL, 500 μL, 600 μL, 700 μL, 800 μL, 900 μL, 1,000 μL, or more of the blood. The container can be configured to hold at most about 1,000 μL, 900 μL, 800 μL, 700 μL, 600 μL, 500 μL, 400 μL, 300 μL, 200 μL, 100 μL, 50 μL, 10 μL, 1 μL, or less of the blood. The desired volume of blood to be collected in the container can be at least about 1 μL, 5 μL, 10 μL, 20 μL, 30 μL, 40 μL, 50 μL, 60 μL, 70 μL, 80 μL, 90 μL, 100 μL, 110 μL, 120 μL, 130 μL, 140 μL, 150 μL, 200 μL, 300 μL, 400 μL, 500 μL, 600 μL, 700 μL, 800 μL, 900 μL, 1,000 μL, or more. The desired volume of blood to be collected in the container can be at most about 1,000 μL, 900 μL, 800 μL, 700 μL, 600 μL, 500 μL, 400 μL, 300 μL, 200 μL, 100 μL, 50 μL, 10 μL, 1 μL, or less.

The coupling unit can comprise at least 1, 2, 3, 4, 5, 6, 7, 8, 9, 10, or more fluidic pathways. The coupling unit can comprise at most 10, 9, 8, 7, 6, 5, 4, 3, 2, or 1 fluidic pathway. An individual fluidic pathway can be disposed adjacent to a surface of the coupling unit. In an example, the coupling unit can comprise the individual fluidic pathway (e.g., an opening or a channel) prior to coupling of the coupling unit to the container (or the container port of the container) of the cartridge assembly. In another example, the coupling unit can comprise at least one groove or an open channel. Upon coupling of the coupling unit to the container port, the groove can be disposed adjacent to a surface of the container (e.g., a surface of the cap), thereby generating an individual fluidic pathway. An individual fluidic pathway can be straight, curved, vertical, diagonal, zigzag (or angular), irregularly shaped, or mixed. An individual fluidic pathway can have a cross-section that is circular, elliptical, oval, triangular, square, rectangular, pentagonal, hexagonal, or any partial shape or combination of shapes thereof. When a plurality of fluidic pathways is provided, each of the plurality of fluidic pathways can have the same shape, thickness, length, width, depth, volume, or surface area. In other cases, two fluidic pathways of the plurality of fluidic pathways may not and need not have the same shape or dimension.

In some embodiments, the cartridge assembly or the sample acquisition device can comprise a separate air venting container configured to trap air that is expunged out of the container through the fluidic pathway(s).

As illustrated in FIG. 5A, the cartridge assembly can comprise the cartridge holder 540 configured to support the container 520. In some cases, a portion of the cartridge holder 540 can be configured to extend outside of the cartridge chamber when the cartridge assembly is coupled to the cartridge chamber. As such, the user can hold onto the cartridge holder 540 (e.g., by holding onto the cartridge tab 542) to insert the cartridge assembly into the sample acquisition device or remove the cartridge assembly from the sample acquisition device. In alternative embodiments, the cartridge holder may not or need not extend outside of the cartridge chamber when the cartridge assembly is coupled to the cartridge chamber. In such cases, the cartridge holder can be hidden (e.g., by a mechanical gate, a motorized gate, or cover of the sample acquisition device), disposed flat relative to the surface of the sample acquisition device, or pressed into the sample acquisition device. In some examples, the cartridge assembly can be releasably coupled to the sample acquisition device using any of the coupling mechanisms described herein. By pressing upon the cartridge holder or a switch on the sample acquisition device, the coupling mechanism can be partially or completely deactivated to allow the holder to protrude relative to the surface of the sample acquisition device, thereby allowing the user to hold onto the cartridge holder 540 to remove the cartridge assembly from the sample acquisition device.

In some embodiments, the cartridge holder 540 can comprise a sealant 544 (e.g., a seal, gasket, liner, ring, etc.) that is configured to hermetically seal the cartridge chamber of the sample acquisition device when the cartridge assembly 500 is coupled to the cartridge chamber. In some cases, the sealant can be disposed on a flat surface of the holder. In alternative cases, the sealant can be disposed on an indent (e.g., a groove) of the holder, such that the outer surface of the sealant is exposed. In some cases, the sealant can be an elastomer gasket. Examples of the elastomer material can include, but are not limited to, any rubber or rubber-like material such as polyisoprenes, butadienes, styrenebutadienes, acylonitrile butadienes, polychloroprenes, isobutylene isoprenes, polysulfides, polymethanes, chlorosulfonated polyethylenes, ethylene propylenes, fluoroelastomers, polysiloxanes, polyesters, polymethanes, silicones, thermoplastic elastomers, and the like.

FIG. 5B shows side sectional views of the sample acquisition device 100 operatively coupled to the cartridge assembly 500, in accordance with some embodiments. The container 520 can be configured to receive the blood flowing into the container 520 in a first direction 524. The one or more fluidic pathways 516 can be configured to direct and expunge the air out of the container 520 in a second direction 526 that is different from the first direction. The angle between the first direction and the second direction can be greater than zero degree and less than 180 degrees. The angle between the first direction and the second direction can be greater than at least 0 degree, 1 degree, 5 degrees, 10 degrees, 15 degrees, 20 degrees, 30 degrees, 40 degrees, 50 degrees, 60 degrees, 70 degrees, 80 degrees, 90 degrees, 100 degrees, 110 degrees, 120 degrees, 130 degrees, 140 degrees, 150 degrees, 160 degrees, 170 degrees, 175 degrees, or more. The angle between the first direction and the second direction can be less than at most 180 degrees, 170 degrees, 160 degrees, 150 degrees, 140 degrees, 130 degrees, 120 degrees, 110 degrees, 100 degrees, 90 degrees, 80 degrees, 70 degrees, 60 degrees, 50 degrees, 40 degrees, 30 degrees, 20 degrees, 10 degrees, 5 degrees, 1 degree, or less. In an example, the first direction and the second direction can be substantially opposite to each other. In another example, the first direction and the second direction can be substantially orthogonal to each other.

As illustrated in FIG. 5B, the sample acquisition device 100 can comprise a port 175. The coupling unit 510 of the cartridge assembly 500 is configured to couple to the port 175 and to the container port 530 of the cartridge assembly 500. The coupling unit 510 can comprise a protrusion (e.g., a tube or extruded feature) configured to couple to the container port 530 of the cartridge assembly 500. The protrusion can be in fluidic communication with the collection tube 535 of the container 520 via the container port 530. Alternatively, the protrusion can penetrate through the container port 530 to be in direct fluidic communication with the collection tube 535. In some cases, a proximal end (e.g., the end opposite of the collection tube 535) of the protrusion can be coupled to a terminal end of the coupling unit. The protrusion can have a cross-section that is circular, elliptical, oval, triangular, square, rectangular, pentagonal, hexagonal, or any partial shape or combination of shapes thereof. The cross-section of the protrusion can be symmetrical or asymmetrical. For example, a diameter of the fluid pathway of the protrusion (e.g., inner diameter of a cannula) can decrease towards the end of the protrusion. Examples of the protrusion can include, but are not limited to, a needle, a tube, a cannula, an open dilator, a nozzle, etc. In an example, the protrusion can be a cannula (e.g., an overmolded cannula) to increase the strength of the protrusion or decrease the thickness of the protrusion. In another example, the protrusion can be a needle (e.g., an overmolded non-coring needle), and the cartridge assembly 500 may not or need not include the valve 532. In such absence of the valve 532, the cartridge assembly can utilize the fluidic pathway 516 for air venting. Alternatively or in addition to the above embodiments, the coupling unit 510 or the container port 530 can comprise a separate opening (e.g., at least 1, 2, 3, 4, 5, or more needles) for air venting. Alternatively or in addition to, the container 520 can include a semi permeable membrane configured to permit air or other gases to vent while preventing liquids from passing through.

FIG. 5C shows perspective views of a flow meter 170 of the sample acquisition device 100 operatively coupled to the cartridge assembly 500, in accordance with some embodiments. The flow meter can comprise a transparent or semi-transparent window (e.g., a visual metering window) that permits a user to observe a progress of the liquid blood collection. When the cartridge assembly is operatively coupled to the sample acquisition device, at least a portion of the collection tube 535 of the container 520 can be aligned with the flow meter 170. Additional details about the flow meter are described elsewhere herein. As abovementioned, at least a portion of the collection tube 535 can be transparent or semi-transparent to allow viewing of the progress of the liquid blood collection into the cartridge assembly 500. Once the blood collection process is complete (e.g., indicated by the indicator(s) 522 or one or more sensors operatively coupled thereto), the cartridge assembly 500 can be removed from the device 100, and at least a portion of the cartridge assembly 500 can be coupled to (e.g., inserted into) the transport sleeve 200 for, e.g., subsequent storage or transportation. In some examples, the entire cartridge assembly 500 can be removed from the device 100 and then inserted into the transport sleeve 200. In other examples, the coupling unit 510 can remain coupled to the device 100, while the rest of the cartridge assembly 500 is decoupled from the coupling unit 510 to be inserted into the transport sleeve 200. In other examples, the entire cartridge assembly 500 can be removed from the device 100, and the coupling unit 510 can be subsequently decoupled from the cartridge assembly 500 for the container 520 to be inserted into the transport sleeve 200. When the coupling unit 510 is decoupled from the container 520 for storage or transportation, the valve 532 and the fluidic pathway 516 can be closed to prevent leakage of the blood. In alternative embodiments, a separate sealant or covering can be applied to the container port 530 to prevent leakage of the blood. The sealant/covering can be configured to protect the collected blood from the outer environment, prior to the insertion of the container into the transport sleeve 200. FIG. 6 shows an example of the cartridge assembly 500 inserted into the transport sleeve 200. Additional details about the transport sleeve are described, e.g., in Section III of the Specification.

In some embodiments, the coupling unit 510 can be coupled to the container 520 during assembly of the cartridge assembly 500. In alternative embodiments the coupling unit 510 can be coupled (temporarily or permanently) to the port within the cartridge chamber of the device during assembly. In alternative embodiments, the coupling unit 510 can be coupled to the container 520 by the user. In some embodiments, upon coupling the cartridge assembly 500 to the device, the force connecting the coupling unit 510 to the port (e.g., the cartridge port) in the device can be greater than the frictional force between the coupling unit 510 and the container port 530, such that the coupling unit 510 can stay in place (e.g., remain coupled to the sample acquisition device) even when the container 520 is pulled away and decoupled from the sample acquisition device. In alternative embodiments, the force connecting the coupling 510 unit to the device can be less than the frictional force between the coupling unit 510 and the container port 530, such that the coupling unit 510 can be decoupled from the device when the container 520 is pulled away and decoupled from the device.

In some embodiments, at least a portion of the cartridge assembly that comes in contact with the collected blood (e.g., the pathway 514 of the coupling unit 510, the valve 532, the container port 530, the inner surface of the collection tube 535, the indicator(s) 522 or any sensors operatively coupled thereto) can be coated with any protective agent disclosed herein. For example, the collection tube 535 can contain or be coated with a substance, such as heparin or EDTA, to help stabilize the collected blood.

In some embodiments, the cartridge assembly can be further configured to selectively separate any number of components of the collected liquid blood, such as, for example, cells, plasma, serum, platelets, specific cell types, DNA (e.g., tumor cfDNA), RNA, protein, inorganic materials, drugs, or any other components. For example, the cartridge assembly 500 can comprise one or more components of the cartridge assembly 300, 400 (e.g., the blood separation membrane 322, the collection media 324, the pre-filter 326, etc.) as described herein, to selectively separate serum or plasma from the collected blood. The cartridge assembly 500 can be configured to selective separate the serum or plasma while the blood is being collected into the cartridge assembly 500, or subsequent to the collection of the blood into the cartridge assembly 500.

Another aspect of the present disclosure provides a system for collecting and storing blood (e.g., liquid blood) from a subject. The system can comprise any of the sample acquisition devices (e.g., the sample acquisition device) and the cartridge assembly (e.g., the cartridge assembly 500, as illustrated in FIGS. 5A-5C) disclosed herein. For example, in some embodiments, the sample acquisition device of the subject system can comprise an onboard vacuum.

Another aspect of the present disclosure provides a method for collecting blood. The method can comprise using any of the sample acquisition devices (e.g., the sample acquisition device) disclosed herein to collect the blood from the subject. The method can further comprise using any of the cartridge assemblies (e.g., the cartridge assembly 500, as illustrated in FIGS. 5A-5C) disclosed herein to receive the blood the subject from the sample acquisition device. In some embodiments, the cartridge assembly can be used to store the blood as liquid blood.

### 3. Modular Sample Chamber

Further aspects of the present disclosure provide a sample chamber for storing a sample (e.g., blood) collected from a subject. The sample chamber can be modular. Such modular sample chamber can be referred to as a “modular sample chamber assembly” or “modular chamber assembly”, as used interchangeably herein. The modular chamber assembly can be operatively coupled to any sample acquisition device (also referred to as a sample acquisition device) disclosed herein, e.g., the device 100 as illustrated in FIG. 1. In some embodiments, the modular chamber assembly can comprise an inlet port configured to couple to a body (or base) of a sample acquisition device. In some cases, the body of the sample acquisition device can comprise a cartridge chamber. The modular chamber assembly can comprise a housing (e.g., a chamber) configured to couple to the inlet port. In some embodiments, an enclosure can be formed within the modular chamber assembly when the chamber is coupled to the inlet port. The enclosure can be configured to support therein at least one cartridge assembly of a plurality of different cartridge assembly types. The plurality of different cartridge assembly types can permit the blood to be collected, processed, or stored in a plurality of different formats. The plurality of different formats can comprise plasma, serum, dried blood, liquid blood, or coagulated blood. In some embodiments, the chamber of the modular chamber assembly or a component therewithin (e.g., an individual cartridge assembly of the plurality of different cartridge assembly types) can utilize one or more components of any of the sample chamber (e.g., treatment/stabilization unit 320 in FIG. 3) described herein. In some embodiments, the inlet port can be a part of a cap that seals the modular chamber assembly. In some embodiments the modular chamber assembly may not and need not include a cartridge assembly and a sample can be collected directly into the enclosure, e.g., as described in the sample chamber 500 in FIG. 5A.

In some embodiments, a portion of the chamber of the modular chamber assembly can be configured to extend out of the base of the sample acquisition device when the inlet port is coupled to a mating feature of the sample acquisition device (e.g., a protrusion 975 as illustrated in FIG. 8B). The portion of the chamber that is extended out of the sample acquisition device can be used as a handle for the user to hold on to the modular chamber assembly, during insertion of the modular chamber assembly into the sample acquisition device, and during removal of the modular chamber assembly from the sample acquisition device. In alternative embodiments, the entire chamber of the modular chamber assembly can be configured to be inserted into the base of the sample acquisition device. In such cases, the chamber of the modular chamber assembly may not be visible when the modular chamber assembly is operatively coupled to the sample acquisition device.

In some embodiments, the inlet port of the modular chamber assembly can comprise a port configured to seal the enclosure. In some cases, the port can be a pierceable port (e.g., a pierceable self-sealing port) that is configured to hermetically seal the enclosure. In some cases, the sealant can be an elastomer gasket. Examples of the elastomer material can include, but are not limited to, any rubber or rubber-like material such as polyisoprenes, butadienes, styrenebutadienes, acylonitrile butadienes, polychloroprenes, isobutylene isoprenes, polysulfides, polymethanes, chlorosulfonated polyethylenes, ethylene propylenes, fluoroelastomers, polysiloxanes, polyesters, polymethanes, silicones, thermoplastic elastomers, and the like. In some examples, the inlet port comprising the pierceable self-sealing port can be a cap of the modular chamber assembly.

In some embodiments, the inlet port of the modular chamber assembly can be configured to couple to at least one cartridge assembly. In an example, the inlet port can be a cap, as disclosed herein, and the cap can be coupled to the cartridge assembly. Such coupling can enclose the cartridge assembly within the modular chamber assembly. In some cases, the cartridge assembly can be configured to be coupled (e.g., releasably coupled) to an inner portion of the modular chamber assembly (e.g., within a sample tube), and the cap can further couple to the cartridge assembly. The inlet port can be in fluidic communication with the cartridge assembly, such that the sample retrieved from the subject by the sample acquisition device can be collected through the inlet port and into the cartridge assembly that is inside the modular chamber assembly. Alternatively, while the inlet port can be coupled to the cartridge assembly, the cartridge assembly can be configured to be in direct fluidic communication with the base of the sample acquisition device to collect the sample from the subject. The inlet port and the cartridge assembly can be coupled to each other using any of the coupling mechanisms described herein. In alternative embodiments, the inlet port and the cartridge assembly can be indirectly coupled to each other via one or more connecting channels or coupling units.

In some embodiments, the plurality of different cartridge assembly types can comprise two or more of the following: (1) a first cartridge assembly type configured to separate the plasma or serum from the collected blood, (2) a second cartridge assembly type configured to collect and store the liquid blood, (3) a third cartridge assembly type configured to hold one or more matrices for collecting and storing the blood as the dried blood, or (4) a fourth cartridge assembly type configured to store coagulated blood. In some cases, the plurality of different cartridge assembly types can comprise three or more of the following: (1) a first cartridge assembly type configured to separate the plasma or serum from the collected blood, (2) a second cartridge assembly type configured to collect and store the liquid blood, (3) a third cartridge assembly type configured to hold one or more matrices for collecting and storing the blood as the dried blood, or (4) a fourth cartridge type configured to store coagulated blood. In some cases, the plurality of different cartridge types can comprise: (1) a first cartridge assembly type configured to separate the plasma or serum from the collected blood, (2) a second cartridge assembly type configured to collect and store the liquid blood, (3) a third cartridge assembly type configured to hold one or more matrices for collecting and storing the blood as the dried blood, and (4) a fourth cartridge assembly type configured to store coagulated blood. The plurality of different cartridge types can have the same shape, thickness, length, width, depth, volume, or surface area. Alternatively, the plurality of different cartridge types may not or need not have the same shape or dimension.

In some embodiments, the modular chamber assembly can be configured to be released and detached from the sample acquisition device when the inlet port is decoupled from the mating feature of the sample acquisition device. Upon decoupling from the base or body of the sample acquisition device, the inlet port can be sealed (e.g., the pierceable self-sealing port can be closed) to protect the collected sample in the cartridge assembly from the ambient environment and/or to protect users or other who can handle the modular chamber assembly. When in use, the modular chamber assembly can be coupled to the sample acquisition device, and a protrusion (e.g., a needle) of the sample acquisition device can penetrate through the inlet port to establish fluidic communication with at least the cartridge assembly of the modular chamber assembly. After sample collection, the modular chamber assembly can be de-coupled from the sample acquisition device, and the inlet port can be closed by self-sealing, e.g., via use of a self-healing or self-enclosing polymer. Alternatively, a separate cap can be applied to the inlet port of the modular chamber assembly to seal and protect the collected sample in the cartridge assembly.

In some embodiments, the modular chamber assembly can be configured to be released and detached from the sample acquisition device after the sample (e.g., the blood of the subject) is collected, processed, or stored on the cartridge assembly of the modular chamber assembly. In some cases, the modular chamber assembly can be released and detached from the sample acquisition device manually by the user, e.g., via one or more switches operatively coupled to the sample acquisition device or the modular chamber assembly. The user can track the collection or processing of the blood through a transparent or semi-transparent window of the modular chamber assembly. The window can be directly exposed to the user (as illustrated in FIG. 8A), or partially or entirely covered by a flow meter of the sample acquisition device (as illustrated in FIG. 1A). Alternatively, the modular chamber assembly can comprise one or more sensors configured to detect (1) the presence of the collected blood, (2) the amount (e.g., volume) of the collected blood, or (3) progress of processing of the blood (e.g., serum/plasma separation). The sensor can be operatively coupled to the coupling/decoupling mechanism between the sample acquisition device and the modular chamber assembly, e.g., any coupling/decoupling mechanism between the sample acquisition device and the inlet port of the modular chamber assembly. The sensor can be any of the sensors as described elsewhere herein.

As abovementioned, a coupling of the inlet port and the chamber can form an enclosure within the modular chamber assembly. In some embodiments, the enclosure can be configured to protect the cartridge from an external environment, after the blood is collected, processed, or stored on the cartridge assembly, and after the modular chamber assembly is released and detached from the sample acquisition device. The enclosure of the modular chamber assembly can serve as or utilize one or more components of any transport sleeve as disclosed herein, e.g., as described in Section III of the Specification. Thus, in some examples, the inlet port/chamber enclosure itself can be used as a storage/transportation packaging.

In some embodiments, the modular chamber assembly can comprise a single cartridge assembly. In alternative embodiments, the modular chamber assembly can comprise two or more cartridge assemblies, e.g., two or more of the plurality of different cartridge types. In some cases, the modular chamber assembly can comprise at least 2, 3, 4, 5, 6, 7, 8, 9, 10, or more cartridge assemblies. The modular chamber assembly can comprise at most 10, 9, 8, 7, 6, 5, 4, 3, or 2 cartridge assemblies. In some examples, the modular assembly can be coupled to two cartridges of different types (i.e., a first cartridge assembly and a second cartridge assembly of different types). The modular chamber assembly can be configured to (1) direct a first portion of the collected blood into the first cartridge assembly, and (2) direct a second portion of the collected blood into the second cartridge assembly. The transition between collection into the first and second cartridge assemblies can be performed manually (e.g., by the user via a switch operatively coupled to the modular chamber assembly) or automatically (e.g., by one or more sensors as disclosed herein). In some examples, the plurality of cartridge assemblies can be coupled in tandem, e.g., forming a fluidic communication from the sample acquisition device, to the first cartridge assembly, and to the second cartridge assembly.

In some embodiments, the cartridge assembly can be releasably coupled to the chamber of the modular chamber assembly, such that the cartridge assembly can be released from the chamber. In some cases, the modular chamber assembly can be re-usable with a new cartridge assembly. For example, the modular chamber assembly can be used more than once, for example twice, three, four, five, five, six, seven, eight, nine, ten or more times by removing a previously used cartridge assembly and installing a new cartridge assembly from the plurality of different cartridge assembly types. In some cases, the modular chamber assembly can be under vacuum prior to coupling to the sample acquisition device. In such cases, upon installation of a new cartridge assembly, vacuum can be established within the modular chamber assembly by use of a separate vacuum device prior to use of the reusable modular chamber assembly comprising the new cartridge assembly.

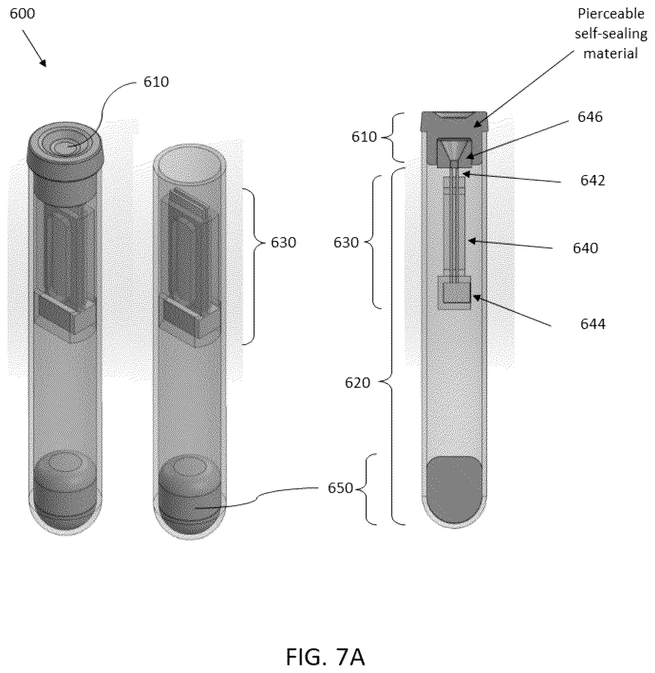

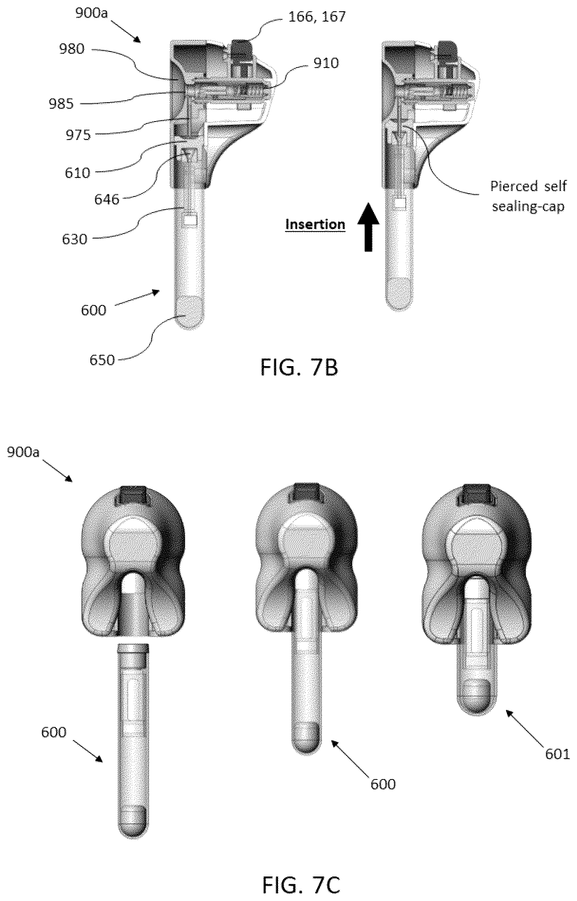

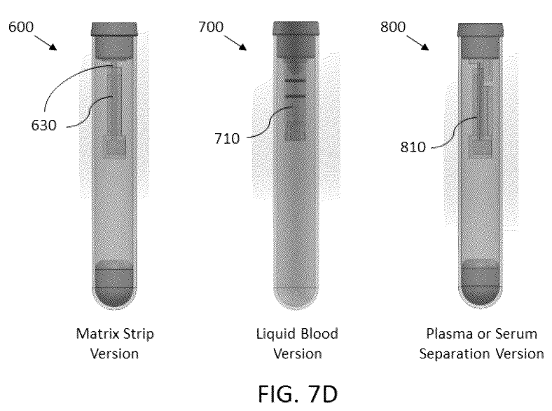

FIGS. 7A-7D illustrates different embodiments of the modular chamber assembly as disclosed herein. FIG. 7A shows perspective views (left two) and a side sectional view (rightmost) of a modular chamber assembly 600 for sample collection, processing, and storage. The modular chamber assembly 600 can comprise an inlet port 610. In some cases, the inlet port can be a cap. The cap can be a pierceable self-sealing cap. The cap can be removable from the rest of the modular chamber assembly. The modular chamber assembly 600 can further comprise a chamber 620 (e.g. a tube or a tube assembly). The chamber 620 can comprise a cartridge assembly 630. The cartridge assembly can include one of a plurality of different cartridge assembly types that permit the blood to be collected, processed, or stored in a plurality of different formats. The plurality of different formats can comprise plasma, serum, dried blood, liquid blood, or coagulated blood. For example, the cartridge assembly 630 can comprise a cartridge 640. The cartridge 640 can comprise one or more matrix strips 642 to absorb and collect the blood or a portion thereof from the subject. The cartridge 640 can also comprise one or more absorbent pads 644 for holding and metering out excess blood. The matrix strip(s) 642 and the absorbent pad(s) 644 can be in fluidic communication with each other. The cartridge assembly can further comprise a connecting port 646 The connecting port 646 can be configured to couple to (e.g., releasably couple to) the inlet port 610 and the cartridge assembly 630. For example, the connecting port can be in fluidic communication with the inlet port and the matrix strip(s) to allow collection of the blood from a sample acquisition device, through the inlet port, and into/onto the matrix strip(s). The connecting port can have various shapes and sizes. For example, the connecting port can be in the shape of a sphere, cuboid, or disc, or any partial shape or combination of shapes thereof. The connecting port can have a cross-section that is circular, elliptical, oval, triangular, square, rectangular, pentagonal, hexagonal, or any partial shape or combination of shapes thereof. In some cases, the connecting port can be pre-assembled or fabricated as part of the inlet port or the cartridge assembly. In an example, the connecting port 646 can be a funnel that serves as a blood flow pathway between the inlet port and the cartridge assembly 630.

In some embodiments, as illustrated in FIG. 7A, the modular chamber assembly 600 can further comprise a desiccant 650 that can be used for drying and/or keeping the samples dry. The desiccant can be disposed within the chamber 620. The desiccant can be a single solid material. The desiccant can include a plurality of desiccant particles. The desiccant particles can be stored within a container (e.g., a pouch).

FIG. 7B illustrates principles of operation and use of the modular chamber assembly and a sample acquisition device for collecting and storing the blood sample from the subject, in accordance with some embodiments. The sample acquisition device 900a can comprise a protrusion or a piercing element 975 (e.g., a needle) configured to penetrate through the inlet port 610 (e.g., a pierceable self-sealing cap), to establish fluidic communication with at least a portion of the modular chamber assembly 600 (e.g., the cartridge assembly 630 comprising the cartridge 640). In some cases, the piercing element 975 can be configured to penetrate through the connecting port 646. Alternatively, as shown on the right image of FIG. 7B, the distal end of the piercing element 975 can be disposed within to but not completely through the connecting port 646 when the modular chamber assembly 600 is coupled to the sample acquisition device 900b, such that the connecting port 646 can receive the collected blood and direct the collected blood into the cartridge assembly 630. Different perspective views of the coupling of the modular chamber assembly 600 to the sample acquisition device 900a are illustrated in FIG. 7C. The modular chamber assembly can have various lengths and/or diameters (as indicated by 600 and 601), and the sample acquisition device 900a can be configured to be compatible with different types and dimensions of the modular chamber assembly. The sample acquisition device 900a can comprise a recess 980 configured to receive the skin of the subject. The recess 980 can comprise an opening 985 configured to allow a piercing element of the lancet 910 to pierce the skin of the subject. The lancet can include a piercing activator 166. The piercing activator can include a button 167.

In some embodiments, the modular chamber assembly or a component thereof (e.g., the cartridge assembly) can be pre-evacuated (e.g., to below ambient pressure) to provide vacuum for the blood draw from the subject. The inlet port (e.g., the cap) can create a seal to maintain vacuum prior to use. In alternative embodiments, vacuum can be provided for the blood draw by the sample acquisition device. In some embodiments, after collecting the sample into the modular chamber assembly, the inlet port can create a seal to maintain the environment within the modular chamber assembly during storage/transportation.

In some embodiments, the modular chamber assembly can function as a vacuum chamber and/or a deposition chamber (or cartridge chamber, sample chamber, etc.). For example, a complete coupling between the modular chamber assembly and the sample acquisition device, e.g., via fully inserting the modular chamber assembly into the body of the sample acquisition device, can trigger the protrusion (e.g., the needle) of the body of the sample acquisition device to pierce the cap of the modular chamber assembly and activate the vacuum. As such, in this example, the sample acquisition device may not or need not require a separate vacuum actuator button. Coupling and decoupling between the modular chamber assembly and the body of the sample acquisition device can be operated using one hand or both hands. In some cases, a complete coupling between the modular chamber assembly and the sample acquisition device can be indicated by a hard stop, or a marking on the modular device, an audible clicking, or other mechanisms.

As mentioned above, the modular chamber assembly can comprise a chamber configured to support (e.g., couple to) a plurality of different cartridge assembly types for permitting the blood to be collected, processed, or stored in a plurality of different formats. As illustrated in FIG. 7D, the modular chamber assembly 600 can comprise a cartridge assembly 630, which in turn comprises one or more matrix strips 642 that are configured to absorb and collect the blood or a portion thereof from the subject. In another example, the modular chamber assembly 700 can comprise a cartridge assembly, which in turn comprises a container (e.g., a tube collector) 710 that is configured to collect liquid blood. The container 710 can utilize one or more components of the cartridge assembly 500 for collecting liquid sample (as illustrated in FIGS. 5A-5C). Referring to FIG. 7D, a different modular chamber assembly 800 can comprise a cartridge assembly, which in turn comprises one or more blood separation membranes 810 for, e.g., serum or plasma separation and storage. The blood separation membrane(s) 810 can utilize one or more components of the cartridge 300 or 400 for blood separation and collection (as illustrated in FIGS. 3A-3F and FIG. 4).

In some embodiments, the chamber (or housing) of the modular chamber assembly can have various shapes and sizes. For example, the chamber can be in the shape of a sphere, cuboid, or disc, or any partial shape or combination of shapes thereof. The chamber can have a cross-section that is circular, elliptical, oval, triangular, square, rectangular, pentagonal, hexagonal, or any partial shape or combination of shapes thereof. In some cases, the chamber can have the same cross-sectional dimension along the length of the chamber. Alternatively, the chamber can have different cross-sectional dimensions along the length of the chamber. In some examples, the chamber can be in a shape of a tube for compatibility with one or more tools for storage (e.g., a bench top rack) or processing (e.g., a centrifuge for blood separation or standard tube racks). The compatibility can enable the modular chamber assembly to be integrated with automated lab procedures.

The cross-sectional dimeter of the chamber of the modular chamber assembly (e.g., the chamber 620, as illustrated in FIG. 7A) can be referred to as either the outside diameter (OD) or the internal diameter (ID). The cross-sectional diameter can be at least about 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, 50 mm, or more. The cross-sectional diameter of the housing can be at most about 50 mm, 45 mm, 40 mm, 35 mm, 30 mm, 25 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, 0.9 mm, 0.8 mm, 0.7 mm, 0.6 mm, 0.5 mm, or less. The longitudinal length of the chamber (e.g., the chamber 620) can be at least about 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 150 mm, 200 mm, 250 mm, 300 mm, 350 mm, or more. The longitudinal length of the housing can be at most about 350 mm, 300 mm, 250 mm, 200 mm, 150 mm, 140 mm, 130 mm, 120 mm, 110 mm, 100 mm, 95 mm, 90 mm, 85 mm, 80 mm, 75 mm, 70 mm, 65 mm, 60 mm, 55 mm, 50 mm, 45 mm, 40 mm, 35 mm, 30 mm, 25 mm, 20 mm, 15 mm, 10 mm, 9.5 mm, 9 mm, 8.5 mm, 8 mm, 7.5 mm, 7 mm, 6.5 mm, 6 mm, 5.5 mm, 5 mm, 4.5 mm, 4 mm, 3.5 mm, 3 mm, 2.5 mm, 2 mm, 1.5 mm, 1 mm, or less. In some examples, the chamber of the modular chamber assembly can be about 13 mm in diameter and about 100 mm in length, about 13 mm in diameter and about 75 mm in length, about 13 mm in diameter and about 66 mm in length, about 13 mm in diameter and about 50 mm in length, about 16 mm in diameter and about 100 mm in length, about 16 mm in diameter and about 75 mm in length, about 16 mm in diameter and about 50 mm in length, or preferably about 16 mm in diameter and about 46 mm in length. In some preferred embodiments, the length of the chamber of the modular chamber assembly can be at most about 75 mm or less.

In some embodiments, a volume (e.g., a closed or sealed volume) of the enclosed chamber of the sample chamber as disclosed herein (e.g., the modular chamber assembly 600) can be selected to provide sufficient vacuum pressure for sample collection. In some cases, the volume of the enclosed chamber can be designed to provide more vacuum pressure than is needed or required for the sample collection, e.g., to accommodate for pressure loss during shelf storage (e.g., from leaking). In some cases, the volume of the enclosed chamber can be selected based on the type of the collected sample and/or the type of processing of the collected sample, as disclosed herein. For example, the internal volume of the modular chamber assembly can be at least about 1 cubic centimeter (cm³), 1.5 cm³, 2 cm³, 2.5 cm³, 3 cm³, 3.5 cm³, 4 cm³, 4.5 cm³, 5 cm³, 6 cm³ 7 cm³, 8 cm³, 9 cm³, 10 cm³, 11 cm³, 12 cm³, 13 cm³, 14 cm³, 15 cm³, 20 cm³, 25 cm³, or more. The internal volume of the modular chamber assembly can be at most about 100 cm³, 90 cm³, 80 cm³, 70 cm³, 60 cm³, 50 cm³, 45 cm³, 40 cm³, 35 cm³, 30 cm³, 25 cm³, 20 cm³, 15 cm³, 14 cm³, 13 cm³, 12 cm³, 11 cm³, 10 cm³, 9 cm³, 8 cm³, 7 cm³, 6 cm³, 5 cm³, 4.5 cm³, 4 cm³, 3.5 cm³, 3 cm³, 2.5 cm³, 2 cm³, 1.5 cm³, 1 cm³, or less. In some examples, the internal volume of the modular chamber assembly can range from about 5 cm³ to about 8 cm³, from about 6.5 cm³ to about 7.5 cm³, or preferably from about 5.5 cm³ to about 6 cm³.

The cap of the modular chamber assembly (e.g., the inlet port 610, as illustrated in FIG. 7A) can be characterized by having a height and a cross-sectional dimension (e.g., diameter). The height of the cap can be at least about 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 2 mm, 3 mm, 4 mm, 5 mm, 6 mm, 7 mm, 8 mm, 9 mm, 10 mm, 15 mm, 20 mm, 30 mm, or more. The height of the cap can be at most about 30 mm, 20 mm, 15 mm, 10 mm, 9 mm, 8 mm, 7 mm, 6 mm, 5 mm, 4 mm, 3 mm, 2 mm, 1.5 mm, 1.4 mm, 1.3 mm, 1.2 mm, 1.1 mm, 1 mm, 0.9 mm, 0.8 mm, 0.7 mm, 0.6 mm, 0.5 mm, 0.4 mm, 0.3 mm, 0.2 mm, 0.1 mm, or less. The cross-sectional diameter of the cap can be at least about 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 25 mm, 30 mm, or more. The cross-sectional diameter of the cap can be at most about 30 mm, 25 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1.5 mm, 1.4 mm, 1.3 mm, 1.2 mm, 1.1 mm, 1 mm, 0.9 mm, 0.8 mm, 0.7 mm, 0.6 mm, 0.5 mm, 0.4 mm, 0.3 mm, 0.2 mm, 0.1 mm, or less. The cross-sectional diameter of the cap can range from about 0.5 mm to about 1.1 mm, from about 0.8 mm to about 1.4 mm, or preferably from about 0.7 mm to about 1 mm.

In any of the devices, systems, methods, or kits disclosed herein, the sample acquisition device (i.e., the sample acquisition device) can be modular. Such device can be referred to as a “modular sample acquisition device.”. The modular sample acquisition device can comprise one or more components of any sample acquisition device disclosed herein, e.g., the device 100 of FIGS. 1A, 3D, and 5B, and the device 900a in FIGS. 7B and 7C. In use, the modular sample acquisition device can be operatively coupled to any sample chamber disclosed herein, e.g., both non-modular and modular sample chambers.

FIG. 8A shows a perspective view of various components of a modular sample acquisition device 900b, in accordance with some embodiments. In some cases, the device 900b in FIG. 8A can be more compact than the device 100 in FIG. 1B, in that the device in FIG. 8A comprises fewer components for operation and functionality. For example, the device illustrated in FIG. 8A may not and need not require a housing (e.g., the cover 152 in FIG. 1B). Alternatively, the device in FIG. 8A can still include a housing. The device 900b shown in FIG. 8A can include modular components such as the lancing assembly 910 and the base or body 920. The device 900b can be operatively coupled to the modular chamber assembly 600 that contains the cartridge assembly 630. In an example, the device 900b can only require the body (or base) 920 and the lancing assembly 910, along with the modular chamber assembly 600 for collecting a sample from a subject. The modular sample acquisition device 900b can comprise a recess 980 configured to receive the skin of the subject. The recess 980 can comprise an opening 985 configured to allow a piercing element of the lancet assembly 910 to pierce the skin of the subject. The lancet assembly 910 can be similar to the lancet as described in FIG. 1A. For example, the lancet assembly 910 can include a piercing activator 166. The piercing activator can include a button 167. The body of the modular sample acquisition device 900b can comprise a sleeve 990 configured to support or receive a plurality of different configurations of the modular chamber assembly, as disclosed elsewhere in the present disclosure. The sleeve 990 can comprise a cutout 995 to allow a user to view progress of the sample collection into the modular chamber assembly. In an example, the modular chamber assembly 600 shown in FIG. 8A can be configured to function as both (1) a collection unit to collect the sample (e.g., blood or a component thereof) from the subject and (2) a transportation unit for storage/transport of the collected sample, without any need of a separate storage/transport device. The modular chamber assembly shown in FIG. 8C can be provided having a pre-evacuated vacuum, When the modular chamber assembly is coupled to the body of the modular sample acquisition device, the vacuum in the modular chamber assembly can be activated, which draws the skin of the subject into the recess 980 (as shown in FIG. 8B) on the body 920, in preparation for piercing of the skin using lancets in the lancing assembly. FIG. 8C shows a perspective view of the modular sample acquisition device sample acquisition device 900b in absence of the modular chamber assembly. The modular sample acquisition device 900b comprises the lancing assembly 910 that is coupled to the body 920. The body 920 can comprise at least one protrusion 975 configured to penetrate through at least a portion of the modular chamber assembly 600 to make fluid communication between the modular sample acquisition device 900b and the modular chamber assembly 600.

As further illustrated in FIG. 8D, the modular sample acquisition device 900b can comprise the lancing assembly 910 that is operatively coupled to a base/body 920. Briefly, the base 920 can generate contact with the skin of the subject, and the lancing assembly 910 can make an incision on the skin for collection of a sample (e.g., blood) from the subject. The base 920 can comprise a port configured to receive any of the modular chamber assemblies disclosed herein (e.g., the modular assembly 600, 700, or 800). For example, the modular chamber assembly 600 comprising the inlet port 610 (e.g., a pierceable self-sealing cap) and the cartridge assembly 630 can be used in conjunction with the sample acquisition device 900. The modular chamber assembly 600 can be inserted into the device 900, during which the piercing element 975 of the modular sample acquisition device 900 pierces through the inlet port 610 to generate fluidic communication with the connecting port 646 and cartridge assembly 630 of the modular chamber assembly 600. Subsequently, the blood can be collected into the cartridge assembly 630, and can be processed. Following the collection, the modular chamber assembly 600 can be retracted from the device 900 for storage or transportation.

FIG. 8E illustrates principles of operation and use of an example modular sample acquisition device 900b and a modular chamber assembly 600, in accordance with some embodiments. It should be noted that any of the processes described in FIG. 8E can be performed with any of the sample acquisition devices and sample chambers in the present disclosure. Referring to FIG. 8E, the modular chamber assembly 600 can be separately packaged (or provided separately) from the modular sample acquisition device 900b. In some alternative embodiments, the modular chamber assembly 600 can be packaged as a partially coupled unit to the modular sample acquisition device. Whether decoupled or partially coupled, a protrusion (e.g., a needle 975) of the modular sample acquisition device 900b may not penetrate through the modular chamber assembly 600 (e.g., the inlet port 610), to avoid activation of vacuum prior to use/operation. To activate vacuum in the modular sample acquisition device 900b, e.g., via the vacuum pressure from the modular chamber assembly 600, the modular chamber assembly 600 can be fully coupled to the modular sample acquisition device 900b, for example in the direction as indicated by the arrow 1005 in FIG. 8E. After using the system comprising the modular sample acquisition device 900b and the modular chamber assembly 600 to collect and/or process the blood of the subject, the modular chamber assembly 600 can be decoupled from the modular sample acquisition device 900b, for example in the direction as indicated by the arrow 1010. The modular chamber assembly 600 can be configured to protect the collected blood sample during storage or transportation. To retrieve the collected sample (e.g., that is stored on the matrix strips 642) for further processing or analysis (e.g., blood separation, blood testing, genetic screening, etc.), at least the cap (e.g., the inlet port 610) of the modular chamber assembly 600 can be decoupled from the modular chamber assembly 600, for example in the direction as indicated by the arrow 1015, to allow access to the collected sample.

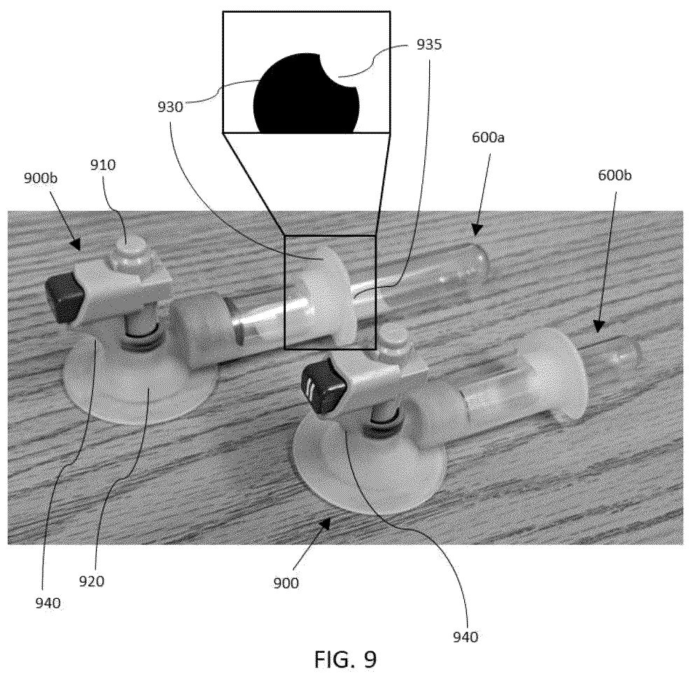

FIG. 9 illustrates an example of the modular sample acquisition device 900b operatively coupled to either a modular chamber assembly 600a or 600b (a cartridge assembly or desiccant not shown). The modular chamber assemblies 600a and 600b can have different dimensions, e.g., different longitudinal lengths. As described herein, the sample acquisition device 900b can comprise the lancing assembly 910 and the base/body 920. The base 920 can be configured to, for example, (1) couple to the lancing assembly 910, (2) contact with the subject's skin (e.g., via a recess or suction cavity of the base 920), and (3) couple to (e.g., releasably coupled to) the modular chamber assembly. The base 920 can comprise a flange 930. A user can use his or her finger(s) to press against the flange 930 to operate the system comprising the modular sample acquisition device 900b and the modular chamber assembly 600a/600b. In some cases, the flange 930 can comprise an indent 935 (e.g., a concave portion) for a finger or thumb of the user's hand to press against for support during use of the modular sample acquisition device and the modular chamber assembly. For example, in a one-handed operation, the user can press his or her thumb against the flange 930 and use one or more other fingers or other portions of the same hand (e.g. the palm) to couple (e.g., push) the modular chamber assembly to the modular sample acquisition device, or decouple (e.g., pull) the modular chamber assembly from the modular sample acquisition device. Alternatively, the user can press his or her thumb against the rest 940 on the body of the modular sample acquisition device 900b and use one or more other fingers or portion of the same hand to couple the modular chamber assembly to the modular sample acquisition device. In some cases, the indent 935 can be disposed on either the left side, middle, or right portion of the flange 930. For example, the position of the indent 935 within the flange 930 can depend on the right-handed or left-handed use (chirality) of the sample acquisition device. In some cases, the handle 930 can comprise more than one indent, e.g., at least 2, 3, 4, 5, or more indents. For example, the flange 930 can include two indents (on both sides of the flange) to be compatible for both left-handed and right-handed operation.

Another aspect of the present disclosure provides a system for collecting and storing blood from a subject. The system can comprise any of the sample acquisition devices described herein (e.g., a modular sample acquisition device and/or a non-modular sample acquisition device). The system can further comprise any of the modular chamber assemblies or other types of sample chambers described herein. In some embodiments, the sample acquisition device can comprise an onboard vacuum. Such vacuum can be sufficient to pull the subject's skin towards the sample acquisition device, to draw blood from the subject when the skin is pierced. In alternative embodiments, the modular chamber assembly can be pre-packaged with onboard vacuum, and the venting of such vacuum into the other portions of the sample acquisition device can be sufficient to pull the subject's skin towards the sample acquisition device, to draw blood from the subject when the skin is pierced.

Another aspect of the present disclosure provides a method (e.g., for blood collection, processing, or storage). The method can comprise using any of the sample acquisition devices (e.g., a modular sample acquisition device and/or a non-modular sample acquisition device) described herein to collect the blood from the subject. The method can further comprise using any of the modular chamber assemblies or other types of sample chambers described herein to collect, process, or store the blood in one or more of a plurality of different cartridge assembly types.

Another aspect of the present disclosure provides a kit comprising any of the sample acquisition devices (e.g., a modular sample acquisition device and/or a non-modular sample acquisition device) described herein, any of the modular chamber assemblies described herein, and any of the plurality of different cartridge assembly types described herein. The kit can comprise at least 2, 3, 4, 5, 6, 7, 8, 9, 10, 15, 20, or more cartridge assemblies. The kit can comprise at most 20, 15, 10, 9, 8, 7, 6, 5, 4, 3, or 2 cartridge assemblies.

### F. Flow Meter

In some embodiments, the device can include a flow meter 170 on the housing, as shown in FIG. TA. The flow meter can be interchangeably referred to herein as a metering window (or metering windows). The flow meter can enable a subject or a user to monitor a progress of the fluid sample collection (e.g., blood sample collection) in real-time as the fluid sample is collected into the sample chamber. For example, the user (e.g., the subject) can rely on the flow meter to determine whether the fluid sample collection is complete or near completion. In some embodiments, the flow meter can be provided on the housing base 110. For example, the flow meter can be a part of, or integrated into the lid of the housing base. The flow meter can be in proximity to the deposition chamber (or cartridge chamber). The flow meter can be located directly above the deposition chamber (or cartridge chamber). The flow meter can be substantially aligned with at least a portion of the sample chamber (e.g., the cartridge 182 of the cartridge assembly) when the sample chamber is inserted into the cartridge chamber.

In some embodiments, the flow meter 170 can include a plurality of windows disposed parallel to a longitudinal axis of the sample chamber. The plurality of windows can include three, four, five or more windows. The windows can be made of an optically transparent material that allows the user (e.g., the subject) to see the underlying matrices in the cartridge. The sample (e.g., the fluid sample) that is collected on the matrices can be visible through the windows. The fluid sample and the matrices of the cartridge can have different colors, preferably highly contrasting colors to permit easy viewing of the flow of the fluid sample along the matrices. The color of the fluid sample (e.g. red color for blood) can sequentially fill each window as the fluid sample is being collected on the matrices in the cartridge. Each window can be indicative of a known amount of fluid sample that is collected. In some alternative embodiments (not shown), the flow meter can include one or more visible markers. The visible markers can replace the windows of the flow meter or can be used in conjunction with the metering windows. The visible markers can be viewable to the naked eye. A visible marker can include an image, shape, symbol, letter, number, bar code (e.g., 1D, 2D, or 3D barcode), quick response (QR) code, or any other type of visually distinguishable feature. A visible marker can include an arrangement or sequence of lights, including LED lights, that can be distinguishable from one another.

In some instances, the visible markers can emit heat or other IR spectrum radiation, UV radiation, radiation along the electromagnetic spectrum. In another example, the sample acquisition device or flow meter can emit vibrations or sounds of different frequencies, pitches, harmonics, ranges, or patterns of sounds that can be detected by the user. For example, the sounds can include words, or musical tones. The vibrations/sounds can be discernible by the human ear. The vibrations/sounds can be used to indicate a progress of the fluid sample collection process. For example, a first vibration/sound can be generated when the fluid sample starts flowing onto the matrices, and a second vibration/sound different from the first can be generated when the fluid sample has completely filled the matrices.

In some embodiments, the flow meter can be used to detect (e.g. enable the user such as the subject to view) a feature, colorimetric change, display of a symbol, masking of a symbol, or other means of indicating the progress of the fluid sample collection, and to indicate that the fluid sample collection has been completed.

In some embodiments, one or more graphical user interfaces (GUIs) can be provided on the sample acquisition device and/or the sample chamber. The GUIs can complement the use of the flow meter. In some embodiments, the function of the flow meter can be incorporated into the GUIs. The GUIs can be rendered on a display screen on the device. A GUI is a type of interface that allows users to interact with electronic devices through graphical icons and visual indicators such as secondary notation, as opposed to text-housing based interfaces, typed command labels or text navigation. The actions in a GUI can be performed through direct manipulation of the graphical elements. In addition to computers, GUIs can be found in hand-held devices such as MP3 players, portable media players, gaming devices and smaller household, office and industry equipment. The GUIs can be provided in a software, a software application, etc. The GUIs can be provided through a mobile application. The GUIs can be rendered through an application (e.g., via an application programming interface (API) executed on the device). The GUIs can allow a user to visually monitor the progress of the sample collection. In some embodiments, the GUIs can allow a user to monitor levels of analytes of interest in the collected sample.

In some embodiments, the sample acquisition device and/or the sample chamber can be capable of transmitting data to a remote server or mobile devices. The data can include for example, user details/information, the date/time/location at which the sample is collected from the subject, the amount/volume of sample collected, time taken to complete the sample collection, maximum/minimum/average flowrates during sample collection, position of the subject's arm during sample collection, whether any errors or unexpected events occurred during the sample collection, etc. In some cases, the data can be transmitted to a mobile device (e.g., a cell phone, a tablet), a computer, a cloud application or any combination thereof. The data can be transmitted by any means for transmitting data, including, but not limited to, downloading the data from the system (e.g., USB, RS-232 serial, or other industry standard communications protocol) and wireless transmission (e.g., Bluetooth®, ANT+, NFC, or other similar industry standard). The information can be displayed as a report. The report can be displayed on a screen of the device or a computer. The report can be transmitted to a healthcare provider or a caregiver. In some instances, the data can be downloaded to an electronic health record. The data can comprise or be part of an electronic health record. For example, the data can be uploaded to an electronic health record of a user of the devices and methods described herein. In some cases, the data can be transmitted to a mobile device and displayed for a user on a mobile application.

### III. Packaging and Transportation of Cartridge Post Sample Collection

The use of flow meters on the sample acquisition device can allow a user to monitor the progress of the sample collection and to know when the sample collection has been completed. The sample chamber can be removed from the sample acquisition device (e.g., the deposition chamber of the device) by pulling on a portion of the sample chamber (e.g., the cartridge tab). At least a portion of the sample chamber (e.g., a filled cartridge) can be subsequently packaged and transported (e.g., by storing the cartridge or a component thereof in a transport sleeve, as disclosed herein) to an external facility for further processing. For example, the sample can be treated, stabilized and stored. In any of the embodiments described herein, the devices can be configured to collect, treat, and store the sample. Samples drawn by the device can be stored in liquid or solid form. The sample can undergo optional treatment before being stored. Storage can occur on the device, off the device, or in a removable container, vessel, compartment, or cartridge within the device.

In some embodiments, the transport sleeve can be configured to protect or stabilize the collected sample (e.g., the liquid sample, such as the liquid blood). The transport sleeve can create a sealed environment to protect the collected sample prior to testing the collected sample. The sealed environment within the transport sleeve can provide (e.g., create) a preferred/stable condition around the collected sample.

In some cases, the transport sleeve can comprise one or more walls (e.g., a double or triple wall to provide an insulated environment) to prevent ambient conditions from affecting one or more internal conditions (e.g., temperature, pressure, humidity, etc.) of the transport sleeve.

In some cases, the sealed environment comprising the collected sample can be cooled (or heated) to a temperature that increases the stability of the collected sample during storage and/or shipping at ambient temperature or at a shipping temperature. In an example, the transport sleeve can comprise at least one temperature regulator, e.g., a thermoelectric cooling/heating apparatus that utilizes the Peltier effect. In another example, the transport sleeve can comprise at least one chemical ice pack. The ice pack and the cartridge can be contained within the same part of the transport sleeve, or contained in separated parts of the transport sleeve, e.g., two parts that are separated by one or more walls. Examples of the ice pack can include, but are not limited to, a combination of a fluid (e.g., aqueous liquid) and a salt (e.g., ammonium nitrate, ammonium thiocyanate, ammonium chloride, ammonium sulfate, potassium chloride, potassium iodide, potassium nitrate, sodium carbonate, etc.). Depending on the salt, a physical mixture of the fluid and the salt can yield endothermic or exothermic reactions to modulate temperature within the transport sleeve. Activation of the ice pack (e.g., the physical mixing of the fluid and the salt by breaking a barrier between them) can be triggered by insertion of the cartridge to the transport sleeve (e.g., automatically by mechanical means of the transport sleeve) or by the user (e.g., via a switch disposed on the transport sleeve). The physical mixing of the fluid and the salt can be immediate (e.g., in seconds or less than a second). Alternatively, a rate of the physical mixing can be controlled (e.g., by timed release of the salt from capsules, slow-dissolving salt tablets, etc.) to prevent over-cooling or over-heating and/or to extend the temperature-regulating duration.

In some cases, the transport sleeve can comprise of a material with high thermal mass or high specific heat. A temperature of the transport sleeve can be pre-adjusted (e.g., cooled or heated) in a temperature-controlled environment, such as a cooler or an oven. Due to the material with high thermal mass, the transport sleeve can maintain the pre-adjusted temperature for extended periods of time. The temperature can be maintained for even longer periods of time in the presence of additional insulating materials or components. Examples of a high specific heat material can include, but are not limited to, cyanimide, ethyl alcohol, ethyl ether, glycerol, isoamyl alcohol, isobutyl alcohol, lithium hydride, methyl alcohol, sodium acetate, water, ethylene glycol, and paraffin wax.

In some cases, the internal volume of the transport sleeve can be partially or fully evacuated (e.g., to a pressure below ambient pressure) to insulate the liquid blood sample. The internal pressure of the transport sleeve can be adjusted manually by a pressure regulator (e.g., a pump such as a diaphragm pump).

In some cases, one or more graphical user interfaces (GUIs) disclosed herein can be provided on the transport sleeve. The GUIs can complement the use of the transport sleeve. In some embodiments, the function of the transport sleeve can be incorporated into the GUIs. The GUIs can be rendered on a display screen on the transport sleeve. The GUI can enable monitoring of one or more conditions of the transport sleeve (e.g., temperature, pressure, humidity, duration of sample storage via timestamping, etc.). The transport sleeve can comprise one or more cameras, and the GUI can enable visualization of the sample contained within the transport sleeve.

### IV. Additional Embodiments

In some cases, any subject sample chamber (e.g., the cartridge assembly 180, 300, 400, 500, the modular chamber assembly 600, 700, 800, etc.) can be used interchangeable with any subject sample acquisition device (e.g., the sample acquisition device 100).

In some cases, the sample chamber can be configured to perform additional processing steps on the sample (e.g., the blood of the subject). Subsequent to or while the blood is collected into the cartridge assembly (e.g., by using the sample acquisition device), the sample can be treated, stabilized, and/or stored. In some embodiments collection devices, e.g. devices disclosed in the present application, can be configured to collect, treat, and store the sample. Sample drawn by the device can be stored in liquid or solid form. The sample can undergo optional treatment before being stored. Storage can occur on the device, off the device, or in a removable container, vessel, compartment, or cartridge within the device.

A sample acquisition device can be configured to collect, treat, stabilize, and store a collected sample. Additional processing (e.g., treatment, stabilization, and storage) can comprise steps or methods and device components configured for concentrating the sample, adjusting or metering the flow of the sample, exposing the sample to one or more reagents, and depositing the sample on a solid substrate or matrix. Methods for using a sample acquisition device can include steps to perform one or more of the following processes: collection, treatment, stabilization, and storage of the sample. Collection, treatment, stabilization, and storage can be performed within a single device. Treatment can comprise filtration of the sample to separate components or analytes of interest. In some embodiments, the collected sample can be collected, treated, and stabilized prior to transfer to a removable cartridge for storage. In other embodiments, one or more steps comprising collecting, treating, and stabilizing, can occur on a removable cartridge.

The devices, systems, and methods disclosed herein can stabilize sample on a matrix (e.g. blood storage matrix, sample collection matrix, matrix, sample stabilization matrix, stabilization matrix (e.g. RNA Stabilization Matrix, Protein Stabilization Matrix), solid matrix, solid substrate, solid support matrix, or solid support). The matrix can be integrated into the device, or external to the device. In some embodiments the matrix can be incorporated into a cartridge for removal (e.g. after sample collection). In some embodiments the matrix can matrix comprise a planar dimensional that is at least 176 mm². A matrix can be prepared according to the methods of U.S. Pat. Nos. 9,040,675, 9,040,679, 9,044,738, or U.S. Pat. No. 9,480,966, which are all herein incorporated by reference in their entirety.

### A. Device Dimensions

In some embodiments, the devices described herein may comprise one or more matrices. The matrices may have an optimized width to length ratio. The width to length ratio of the one or more matrices may be optimized for collection of a type of sample. For instance, the width to length ratio of the one or more matrices may be optimized to yield the highest amount of plasma separation. The width to length ratio of the one or more matrices may be determined based on a desired hematocrit level (for instance from 15-50%), a desired plasma volume, and/or a desired sample volume. In some cases, the integrated device may comprise one or more matrices that are able to separate blood cells and plasma.

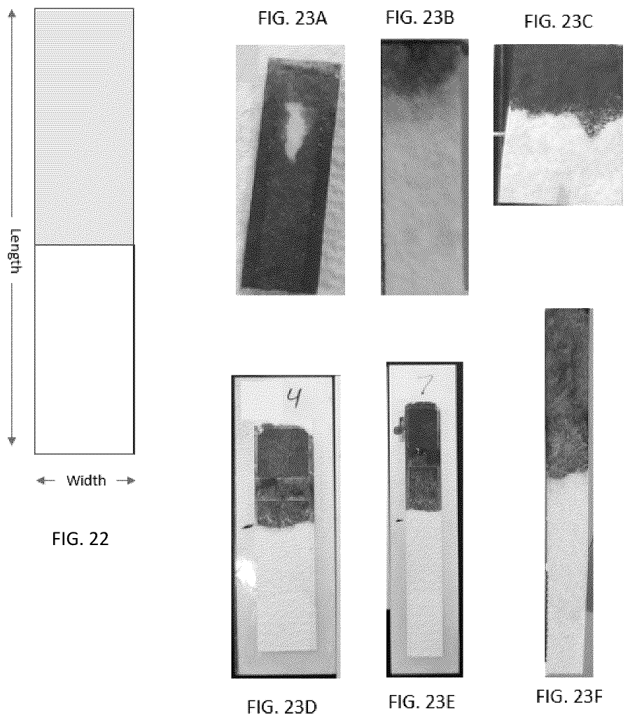

In some cases, a width to length ratio of one or more matrices may be from 1:3 to 1:10. In some cases, a width to length ratio of the one or more matrices may be at least about 1:3. In some cases, a width to length ratio of the one or more matrices may be at most about 1:10. In some cases, a width to length ratio of the one or more matrices may be 1:3 to 1:4, 1:3 to 1:5, 1:3 to 1:6, 1:3 to 1:7, 1:3 to 1:8, 1:3 to 1:9, 1:3 to 1:10, 1:4 to 1:5, 1:4 to 1:6, 1:4 to 1:7, 1:4 to 1:8, 1:4 to 1:9, 1:4 to 1:10, 1:5 to 1:6, 1:5 to 1:7, 1:5 to 1:8, 1:5 to 1:9, 1:5 to 1:10, 1:6 to 1:7, 1:6 to 1:8, 1:6 to 1:9, 1:6 to 1:10, 1:7 to 1:8, 1:7 to 1:9, 1:7 to 1:10, 1:8 to 1:9, 1:8 to 1:10, or 1:9 to 1:10. In some cases, a width to length ratio of the one or more matrices may be about 1:3, 1:4, 1:5, 1:6, 1:7, 1:8, 1:9, or 1:10. In some preferred embodiments, a width to length ratio of the one or more matrices may be from 1:3 to 1:5. A preferred width to length ratio of one or matrices may result in a matrix with a 50:50 whole blood to plasma region. In a preferred embodiment, the matrix may be optimized to stabilize a blood volume from 150-200 μL. For instance, FIG. 22 illustrates a matrix wherein the blood volume applied to the matrix is filtered into cells and plasma. The matrix shown in FIG. 22 may have a width and a length that results in a 50/50 whole blood to plasma region. In a preferred embodiment, the matrix may result in a clear separation of whole blood and plasma. In a preferred embodiment, the matrix may result in a large plasma region wherein the plasma region is filled with highly concentrated plasma.

In some cases, the width to length ratio may be optimized to yield the greatest amount of plasma separation for a given matrix material, range of sample volumes, range of hematocrit levels, and/or a thickness of the matrix material. For a given sample volume there may be an ideal width to length ratio that results in a concentrated but well spread-out plasma area.

In some cases, the optimized device dimensions may result in some membranes that are fully saturated with whole blood and leave no plasma regions (over saturated). In other cases, the membranes may absorb all whole blood and may never separate the plasma (under saturated). In some cases, hemolysis into plasma area may contaminate results. In some cases, a near 50/50 ratio of separated plasma to whole blood may results, with good saturation suggesting a high-yield of plasma for a given region. In some cases, the optimized device dimensions may result in a dense, well spread-out plasma that is both higher quality and easier to process than alternative geometries.

The benefits of optimized device dimensions may include, for example, optimized plasma volume yield per surface area, a larger plasma/whole blood surface area, more plasma spread across a larger area (which makes for a great number of biomarkers that can be analyzed and permits easier extraction of plasma from the card via punching, cutting, etc.), more flexibility to various sample volumes (e.g., between 150-250 uL), more flexibility to various sample hematocrit levels (e.g., between 15-50%), an optimized user experience (e.g., draw times less than 10 minutes), and ease of manufacture. Further, in some cases, after plasma separation the whole blood region (red blood cells) may not be destroyed, and in fact may be preserved enough to extract one or more analytes.

The matrix can be configured to selectively stabilize sample preparation reagents comprising protein and/or nucleic acids. The matrix can be configured to stabilize protein and nucleic acids can comprise an oligosaccharide (e.g. a trisaccharide) under a substantially dry state. The oligosaccharide or trisaccharide can be selected from a group comprising: melezitose, raffinose, maltotriulose, isomaltotriose, nigerotriose, maltotriose, ketose, cyclodextrin, trehalose or combinations thereof. In some embodiments the matrix can comprise melezitose. In further embodiments the melezitose can be under a substantially dry state. In some embodiments, melezitose under a substantially dry state can have less than 2% of water content. In the matrix, the concentration of the melezitose can be in range of about 10% to about 30% weight percent by mass (e.g. calculates as the mass of the solute divided by the mass of the solution where the solution comprises both the solute and the solvent together. The concentration of melezitose can be 15% weight percent by mass. The melezitose can be impregnated in the matrix. In some embodiments, the impregnated melezitose concentration in the matrix results from immersing the matrix in a melezitose solution comprising between about 10 to about 30%. In some other embodiments, 15% melezitose is impregnated into the matrix in a dried state. The matrix can be passively coated or covalently-modified with melezitose. In other embodiments the melezitose can be applied to the surface of the matrix (e.g. with dipping, spraying, brushing etc.). In some other embodiments, the matrix can be coated with a 15% solution of melezitose. In some embodiments the matrix can matrix comprise a planar dimensional with a surface area that is at least 176 mm². The matrix can comprise additional components to stabilize protein and/or nucleic acids, including various stabilization molecules. A non-limiting example of a stabilization molecule is validamycin. In some embodiments the matrix can comprise 31-ETF (e.g. cellulose based matrix) and melezitose.

The matrix can comprise a buffer reagent. A buffer reagent can be impregnated into the matrix. Buffers can stabilize sample preparation reagents and/or various sample components. The matrix can comprise a reagent or compound that minimizes nuclease activity, e.g., a nuclease inhibitor. The matrix can comprise a reagent or compound that minimizes or inhibits protease activity, e.g., a protease inhibitor. A protease inhibitor can be synthetic or naturally-occurring (e.g., a naturally-occurring peptide or protein). The matrix can comprise one or more free radical scavengers. The matrix can comprise a UV protectant or a free-radical trap. The matrix can also comprise oxygen scavengers, e.g. ferrous carbonate and metal halides. Other oxygen scavengers can include ascorbate, sodium hydrogen carbonate and citrus. The matrix can comprise a cell lysis reagent. Cell lysis reagents can include guanidinium thiocyanate, guanidinium hydrochloride, sodium thiocyanate, potassium thiocyanate, arginine, sodium dodecyl sulfate (SDS), urea or a combination thereof. A solid support matrix can comprise a reducing agent.

In some embodiments, the matrix may be a monolithic membrane. In some embodiments, the matrix may be a monolithic matrix able to separate blood cells from plasma and stabilize the blood sample. In some embodiments, the matrix may be a monolithic matrix able to separate blood cells from plasma and stabilize blood cells and plasma. In some cases, the matrix may be treated with a reagent that stabilizes whole blood cells. In some cases, the matrix may be treated with a reagent that stabilizes a blood analyte. In some cases, the matrix may be treated with a reagent that stabilizes plasma. In some cases, the matrix may be treated with a reagent that stabilizes whole blood cells and plasma. In some cases, a first portion of the matrix may be treated with a reagent that stabilizes whole blood cells and a second portion of the matrix may be treated with a reagent that stabilizes plasma. In such an embodiment, the different portions of the matrix may be used to analyze different blood analytes.

In some cases, the matrix may be treated to make it easier to detect stabilized plasma on the membrane. In some cases, the treatment to detect stabilized plasma on a membrane may involve the use of a sensor (e.g., a chemical sensor, a bio sensor, an optical sensor, etc.) or a color modifier. A user's experience may be improved by indicating that a sufficient plasma volume has been collected. A sensor or a color modifier may also be used to assist in preventing an overage of collected sample. The sensor may also assist in automating blood collection.

### B. Geometric Features

In some embodiments, the matrix may comprise one or more geometric features that improve sample collection or stabilization. In some cases, the one or more geometric features may comprise, for example, an intentionally placed relief or squeeze geometry configured to act as a channel to guide the plasma. The dimensions, shapes, and/or physical features of the one or more geometric features may be adjusted for various different use cases and for use with any type of chemical agent. In some cases, the intentionally placed relief or squeeze geometry may provide one or more flow paths to guide the plasma in or through the matrix, and may stop or near-stop plasma flow to intentionally isolate plasma within one or more regions or sections of the matrix.

In some cases, the one or more geometric features may comprise a relief feature. The relief feature may be used to store overflow once enough sample has been collected from the user For instance, the relief feature may be used to store overflow once a pre-determined amount (e.g., mass or volume) of a sample has been collected from the user. An intentional relief such as a tapered neck may prevent hemolysis by slowing the red cells from intruding upon the plasma region and further squeezing as much plasma from the blood as possible. A relief feature may also assist in separating different collection regions of the matrix and may allow the matrix to be used for analyzing multiple analytes from the same matrix.

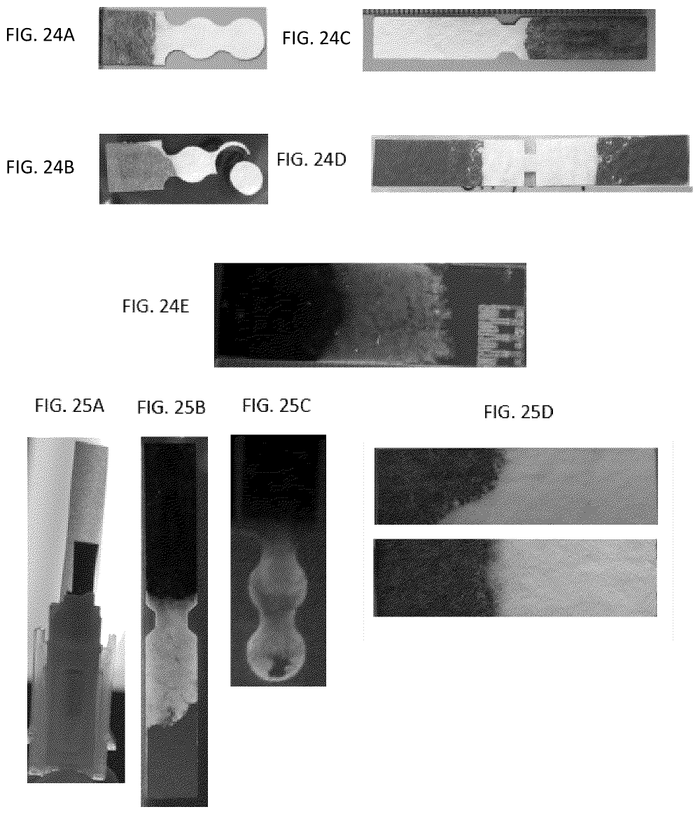

In some cases, the one or more geometric features may comprise a feature configured to assist with “squeezing” the plasma from the whole blood, thereby further optimizing the plasma yield for an otherwise smaller surface area of the membrane. The feature may squeeze the plasma by applying a mechanical force, or by applying a pressure (e.g., a pressure differential). An example of such a feature that assists with “squeezing” the plasma from the whole blood to further optimize the plasma yield for an otherwise smaller surface area of the membrane are illustrated in FIGS. 24A-E. The geometric feature may be a narrow neck (as shown in FIG. 24A), a perforated area (such as shown in FIG. 24B), a soft notch (greater than 10 degrees, as shown in FIG. 24C), a hard notch (less than 0 degrees or a right angle as shown in FIG. 24D) or a laser etched perforation which may be used to microsample a region of the matrix (as shown in FIG. 24E). In some cases, the one or more geometric features may comprise one or more notches. The one or more notches may be used to stop or near-stop plasma flow to intentionally isolate plasma across regions. In some embodiments, one or more predefined perforated areas can be etched, lasered, or mechanically punched into the matrix material, in order to facilitate end use processing.

In some cases, a taper neck design may help to prevent hemolysis by slowing red region from intruding upon the plasma region and further squeezing as much plasma from the blood as possible. In some cases, a notch may be effective in stopping or drastically slowing flow in or through the matrix material. In some cases, perforation of the matrix material may enhance processing. In some cases, plasma dots and/or other tear-able, well defined areas may provide known plasma quantities that are easy to process. In some cases, laser cutting or die-cutting may be used to generate the geometric features. In any of the embodiments described herein, the geometric features may be easily be used in combination with chemical treatments and/or any of the optimal device dimensions described elsewhere herein.

Benefits of the geometric features may include, for example, the ability to account for overflow scenarios by the collection user leaving the device on too long and/or underflow scenarios where not enough sample is collected. The geometric features may also make it easier to multiplex and process different pieces of the collection materials in various tubes without needing to punch the matrix material. The geometric features may also be used to collect as much plasma in as small of a surface area and/or volume of material as possible. The geometric features disclosed herein may be agnostic to chemical treatments or overall device dimensions, and may be easy to manufacture with high yields.

### C. Treatments

One or more treatments may be performed or applied to the material to make it easier to detect the plasma area visually. The one or more treatments may be used to detect the plasma region using a sensor or some other non-human observation. The treatments may optimize the plasma separation in different ratios based on the intended analytes to be analyzed. The treatment may let a user know when enough plasma has been collected. In some cases, the treatment may stabilize the whole blood region and/or the plasma region for analyte recovery.

In some cases, sugars or surfactants may be added to help optimize the plasma separation. Sugar/surfactant combinations may help with more sharply defining the plasma region. In some cases, certain combinations of pretreatments may reduce hemolysis into the plasma region. In some cases, one or more non-destructive agents may be used to make the plasma region more visibly noticeable. In some cases, a treatment may be added to act as a user notification when it fluoresces a certain color, to let the user know that enough blood has been collected. The fluorescence may have a wavelength ranging from about 100 nanometers to about 900 nanometers. In some cases, the wavelength of fluorescence may be less than about 100 nanometers, or greater than about 900 nanometers.

The treatments may provide several benefits for the end user. For example, the user experience may be greatly improved by indicating the most ideal time to remove the device, thus ensuring the maximum amount of collected sample without overage. The treatments may also assist the lab technician in his or her throughput efficiency for recovering analytes. In some cases, the treatments may allow for more accurate results by utilizing highest quality sample regions. In some cases, the treatments may operate as a visual aide to benefit the user, the lab technician and/or a non-human automation step (e.g., an automated processing step to process the sample). In some embodiments, software and hardware can be specifically designed to work in corroboration with the visual aid for the purpose of pre-peri- and post processing and analysis of the plasma separation membrane.

In some embodiments, sample acquisition and stabilization can require user action to proceed between one or more phases of the sample collection, separation, and optional stabilization process. A system (e.g., the sample acquisition device, the sample chamber, etc.) can require user action to activate sample acquisition, and move sample between separation, stabilization, and storage. Alternatively, user action can be required to initiate sample acquisition as well as one or more additional steps of the sample collection, separation or stabilization process. User action can include any number of actions, including pushing a button, tapping, shaking, rupture of internal parts, turning or rotating components of the device, forcing sample through one or more components (e.g., chambers) and any number of other mechanisms. Movement through the phases can occur in tandem with sample collection, or can occur after sample collection. Anytime during or prior to the processing phases the entire sample or components of the sample can be exposed to any number of techniques or treatment strategies for pre-treatment of cells of biological components of the sample; potential treatment includes but is not limited to treatment with reagents, detergents, evaporative techniques, mechanical stress or any combination thereof.

In some embodiments, the sample acquisition device can be operatively coupled to at least one valve (e.g., a check valve) that couples the sample acquisition device to the sample chamber, and vice versa. At least 1, 2, 3, 4, 5, or more valves can be configured to couple the sample acquisition device to the cartridge assembly. For example, the sample acquisition device 900b and the modular chamber assembly 600, as shown in FIG. 8A, can be coupled to each other via at least one valve. In some cases, the valve can be apart of the sample acquisition device 900b (e.g., fabricated as part of the device), and can be configured to releasably couple to the modular chamber assembly (e.g., to the inlet port 610 of the modular chamber assembly 600, as shown in FIG. 7A). Alternatively, the valve can be coupled to the sample acquisition device prior to coupling of the cartridge assembly through the valve and to the sample acquisition device. During sample collection, the valve can be configured to maintain the suction at the subject's skin by the sample acquisition device, even when the modular chamber assembly is decoupled from the sample acquisition device, thereby allowing replacement of the modular chamber assembly with a second modular chamber assembly. Once the second modular chamber assembly is coupled to the sample acquisition device via the valve, the valve can be opened (e.g., manually or automatically) to continue drawing of the blood through the sample acquisition device and into the second modular chamber assembly.

In some embodiments, the sample acquisition device and the sample chamber (e.g., the modular device 900b and the modular chamber assembly 600, as shown in FIG. 8A) can be configured to be operable by the user. For example, the user can apply the sample acquisition device on the user's skin, and subsequently couple the sample chamber (e.g., the modular chamber assembly 600) to the sample acquisition device. In another example, the user can partially couple the sample chamber to the sample acquisition device (e.g., partial insertion or rotation), apply the sample acquisition device (which is partially coupled to the sample chamber) onto the skin, and subsequently completely couple the sample chamber to the sample acquisition device, e.g., for activation of the blood drawing process. The final coupling can require insertion of the sample chamber into the sample acquisition device, e.g., a longitudinal movement relative to the sample acquisition device. The longitudinal movement can be at least about 0.1 millimeter (mm), 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, or more. The longitudinal movement can be at most about 10 mm, 9 mm, 8 mm, 7 mm, 6 mm, 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, or less. Alternatively or in addition to, the final coupling can require a rotation of the sample chamber relative to the sample acquisition device. The rotational movement can be over an angle of at least about 1 degree, 2 degrees, 3 degrees, 4 degrees, 5 degrees, 6 degrees, 7 degrees, 8 degrees, 9 degrees, 10 degrees, 20 degrees, 30 degrees, 40 degrees, 50 degrees, 60 degrees, 90 degrees, 120 degrees, 150 degrees, 180 degrees, 270 degrees, 360 degrees, or more. The rotational movement can be over an angle of at most about 360 degrees, 270 degrees, 180 degrees, 150 degrees, 120 degrees, 90 degrees, 60 degrees, 50 degrees, 40 degrees, 30 degrees, 20 degrees, 10 degrees, 9 degrees, 8 degrees, 7 degrees, 6 degrees, 5 degrees, 4 degrees, 3 degrees, 2 degrees, 1 degree, or less. In some cases, the final coupling can be configured to activate the protrusion (e.g., the needle) of the sample acquisition device to penetrate into the sample chamber (e.g., penetrate through the inlet port 610 of the modular chamber assembly 600) to activate vacuum in the system (e.g., vacuum transfer from the sample acquisition device to the cartridge assembly, from the cartridge assembly to the sample acquisition device, etc.). In some cases, the lancet of the sample acquisition device can be configured to be activated upon the complete coupling of the sample chamber to the sample acquisition device. Alternatively, the lancet can be pre-activated prior to the complete coupling of the sample chamber to the sample acquisition device.

In some embodiments, the vacuum pressure exerted by the sample acquisition device to the skin of the subject prior to or during the sample collection (e.g., blood draw) can be selected based on one or more conditions, e.g., which portion of the body of the subject the same is to be collected, a desired amount of the sample to be collected, etc. Examples of the conditions of the subject can include, but are not limited to, skin properties (e.g., elasticity, firmness, shape, thickness, wrinkling), gender, age, diseases, a number of previous uses of the device for sample collection, etc. In some examples, a particular type of the sample acquisition device and/or the sample chamber can be selected depending on such condition(s), thereby to yield the desired vacuum pressure for the subject. In alternative embodiments, a set of the sample acquisition device and one or more sample chambers can be configured to provide sufficient vacuum pressure for the sample collection for a plurality of individuals, with minimal or no damage (e.g., bruising) to each individual's skin.

In some embodiments, upon coupling of the sample chamber to the sample acquisition device as disclosed herein (e.g., coupling of the modular chamber assembly 600 to the sample acquisition device 900b), the applied vacuum pressure of the sample acquisition device to the skin of the subject can be less than about −0.5 psig, −0.6 psig, −0.7 psig, −0.8 psig, −0.9 psig, −1 psig, −2 psig, −3 psig, −4 psig, −5 psig, −6 psig, −7 psig, −8 psig, −9 psig, −10 psig, −11 psig, −12 psig, −13 psig, −14 psig, or lower. In some cases, the applied vacuum pressure of the sample acquisition device to the skin of the subject can range from about −1 psig to about −14.7 psig, −1 psig to about −10 psig, preferably from about −2 psig to about −6 psig, or preferably from about −2.5 psig to about −5.8 psig.

In some embodiments, as described in the present disclosure, the sample chamber (e.g., the modular chamber assembly 600) can serve as a vacuum chamber to provide sufficient vacuum to the sample acquisition device for sample collection. In some cases, the initial vacuum pressure of the modular chamber assembly (e.g., prior to coupling to the sample acquisition device) can be dictated or selected by one or more of the following variables: (1) volume of the vacuum chamber, (2) level of vacuum applied to the vacuum chamber, (3) dead volume (e.g., cavity, channels, lancet area) in the sample acquisition device and the cartridge assembly that can be at ambient pressure prior to vacuum activation, (4) age or numbers of previous uses of the sample acquisition device or the modular chamber assembly, or (5) expected shelf-life of the sample acquisition device or the modular chamber assembly. In an example, vacuum can decay over time due to material gas permeability, and thus the applied vacuum pressure to the vacuum chamber (e.g., the modular chamber assembly) can be selected to accommodate for the vacuum decay. In some embodiments, the initial vacuum pressure of the vacuum chamber can be less than about −5 psig, −6 psig, −7 psig, −8 psig, −9 psig, −10 psig, −11 psig, −12 psig, −13 psig, −14 psig, or lower. In some cases, the initial vacuum pressure of the vacuum chamber can range from about −5 psig to about −14.7 psig, preferably from about −10 psig to about −14.7 psig, or preferably from about −12.5 psig to about −14.7 psig.

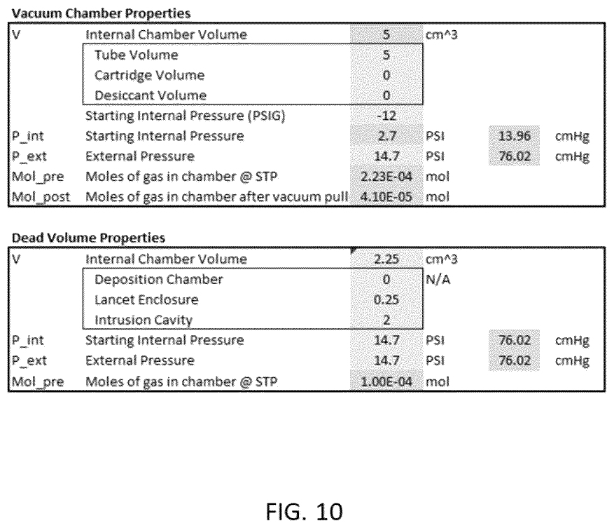

FIG. 10 shows various dimensional and pressure parameters of the sample acquisition device and/or the sample chamber for sample collection, as disclosed herein. For example, the parameters shown in FIG. 10 can be used for the modular chamber assembly as described in FIGS. 7-8. However, the parameters can be applicable (with or without modifications) to other sample acquisition device and sample chamber types. Referring to FIG. 10, the parameters for sample collection can be based on at least the vacuum chamber properties and dead volume properties. The vacuum chamber (e.g., the modular chamber assembly 600) properties can be dependent on one or more parameters comprising: (1) internal chamber volume (V) of the modular chamber assembly that comprises the chamber 620 volume, the cartridge assembly 630 volume, and/or the desiccant 650 volume, (2) the starting internal pressure (P_int) of the chamber, (3) the external pressure (P_ext), (4) the amount of gas in the chamber prior to vacuum pull (Mol_pre), or (5) amount of gas in the chamber after vacuum pull (Mol_post). The dead volume (e.g., cavity, channels, lancet area) properties can be dependent on one or more parameters comprising: (1) internal chamber volume (V) of the sample acquisition device that comprises the deposition chamber, lancet enclosure, and/or the intrusion cavity, (2) the starting internal pressure (P_int), (3) the external pressure (P_ext), or (4) the amount of gas in the chamber of the sample acquisition device (Mol_pre). In an example, based on the parameters and values provided in FIG. 10, a final starting vacuum applied to the skin of the user to initiate the sample collection process can be −5.83 psig.

### D. Blood Separation Assembly

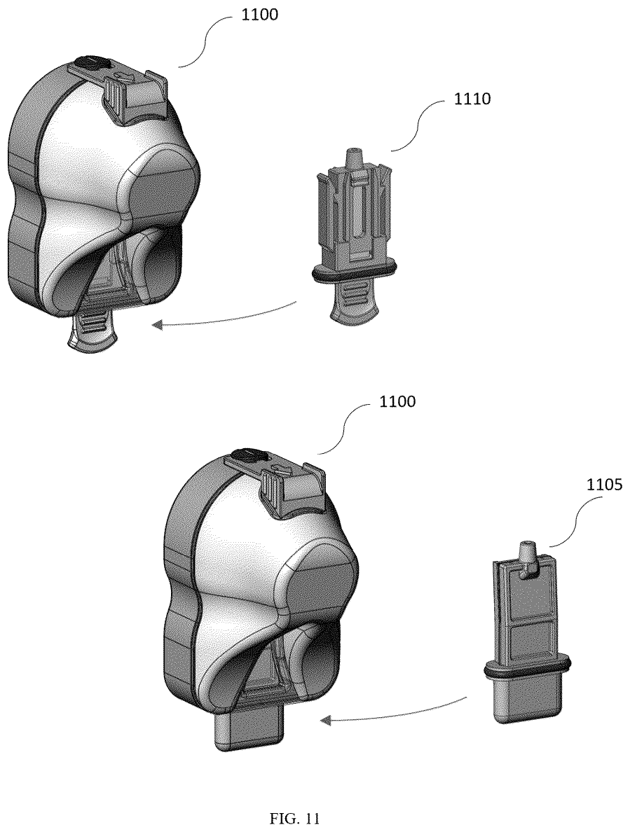

FIG. 11 illustrates an exemplary sample acquisition device 1100 as described herein, which can be used with a cartridge assembly 1110 as described herein and additional cartridge assemblies 1105 as will be discussed. In any of the embodiments disclosed herein, the device can be re-usable. For example, a device can be used more than once, for example twice, three, four, five, five, six, seven, eight, nine, ten or more times. In any of the embodiments disclosed herein, the device can be for single use and may be disposable. In any of the embodiments disclosed herein, the sample acquisition device 1100 can be used with any cartridge assembly as described herein. In specific embodiments, the sample acquisition device 1100 can be used with cartridge assembly 1100 for one use and can be used with cartridge assembly 1105 for another use.

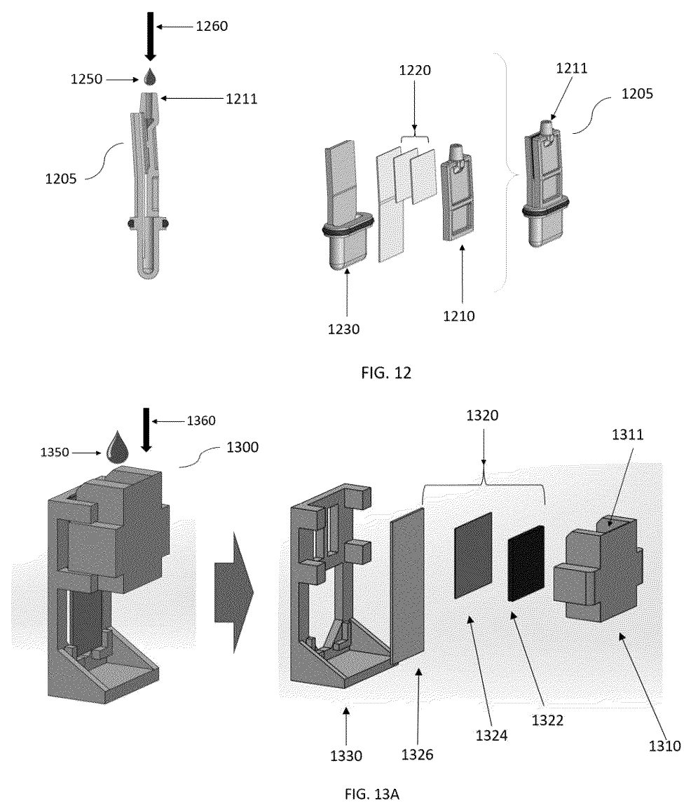

FIG. 12 illustrates a cartridge assembly 1205 which can be used with the sample acquisition device 1100. The cartridge assembly 1205 can be comprised of several components. For example, the cartridge assembly can be comprised of a cartridge 1210, a treatment/stabilization unit 1220, and a cartridge tab 1230. In some embodiments, the treatment/stabilization unit 1220 is supported (e.g. sandwiched) between the cartridge tab 1230 and cartridge 1210. The cartridge tab 1230 may comprise a substrate. In some embodiments, the cartridge tab may be coupled to the substrate. The substrate can be configured to support the treatment/stabilization unit 1220. For example, the perimeter of the substrate may be configured to be substantially the same shape and size of the perimeter of the treatment/stabilization unit 1220. The perimeter of the substrate may also be larger than the perimeter of the treatment/stabilization unit 1220 to ensure the treatment/stabilization unit does not come into contact with the cartridge tab 1230. The cartridge 1210 can be disposed adjacent to the treatment/stabilization unit 1220. In some embodiments, the treatment/stabilization unit 1220 is supported (e.g. sandwiched) between the substrate and the cartridge 1210.

The cartridge assembly can be releasably coupled to the sample acquisition device 1100 and releasably detached from the device. In any embodiments disclosed herein, the cartridge tab 1230 can protrude from an edge of the device. In any of the embodiments disclosed herein, the cartridge tab and the piercing activator/vacuum activator (e.g., buttons 115/167) can be located on different sides (e.g. opposite ends) of the housing. The cartridge assembly 1205 can be releasably coupled to and detachable from the sample acquisition device 1100 as other cartridge assemblies described herein are.

The treatment/stabilization unit 1220 can be comprised of several components in a layered structure. In some embodiments, the components of the treatment/stabilization unit 1220 may include a pre-filter, a separation membrane, and a collection matrix, for example as described elsewhere herein. The pre-filter can be configured to be disposed adjacent to the cartridge 1210 and be the first component of the treatment/stabilization unit that a sample from a subject comes in contact with. The separation membrane may be disposed adjacent to and be sandwiched between the pre-filter and the collection matrix. The collection matrix may be disposed adjacent to and be sandwiched between the separation membrane and the cartridge tab 1230. The cartridge 1210 of the cartridge assembly can be configured to support the components of the treatment/stabilization unit 1220 on which the fluid sample 1250 (e.g., blood) is collected. The cartridge can be configured to support one or more absorbent pads (not shown) for holding excess fluid. The absorbent pads can be configured to rest at the base of the collection matrix of the treatment/stabilization unit 1220. The absorbent pads can absorb excess fluid sample and can help to ensure that a predefined volume of fluid can be collected on each of the components of the treatment/stabilization unit.

The cartridge assembly 1205 can be configured to receive blood from a subject at a blood input area 1211. The blood input area 1211 may be sized and shaped to impact and/or control the volume of sample entering the cartridge assembly. The cartridge assembly can also be configured to receive other types of biological samples that are not blood. Examples of biological samples suitable for use with the devices of the disclosure can include sweat, tears, urine, saliva, feces, vaginal secretions, semen, interstitial fluid, mucus, sebum, crevicular fluid, aqueous humour, vitreous humour, bile, breast milk, cerebrospinal fluid, cerumen, enolymph, perilymph, gastric juice, peritoneal fluid, vomit, and the like. In some embodiments, a fluid sample can be a solid sample that has been modified with a liquid medium. In some instances, a biological sample can be obtained from a subject in a hospital, laboratory, clinical or medical laboratory.

The treatment/stabilization unit can be configured to collect and store blood as dried blood. The cartridge assembly can be configured to receive blood in the blood input area 1211. The cartridge 1210 can be configured in a way that directs the flow of the blood towards the cartridge tab 1230, encouraging the blood to travel through each component of the treatment/stabilization unit. In some embodiments, a direction of flow of the blood through the treatment/stabilization unit can be different from a direction of flow of the blood through the blood input area. In some examples, the direction of flow of blood through the blood input area can be substantially parallel to the longitudinal axis 1260 of the blood separation assembly, and the direction of flow of blood through the treatment/stabilization unit can be different than the longitudinal axis of the blood separation assembly. The direction of flow of blood through the treatment/stabilization unit may not be on the same plane as the longitudinal axis of the blood separation assembly. The direction of flow of blood through the treatment/stabilization unit can be offset by the direction of flow of blood through the blood input area by at least about 5 degrees, 10 degrees, 15 degrees, 20 degrees, 30 degrees, 40 degrees, 50 degrees, 60 degrees, 70 degrees, 80 degrees, 90 degrees, 100 degrees, 110 degrees, 120 degrees, 130 degrees, 140 degrees, 150 degrees, 160 degrees, 170 degrees, 175 degrees, or more. The direction of flow of blood through the treatment/stabilization unit can be offset by the direction of flow of blood through the blood input area by at most about 170 degrees, 160 degrees, 150 degrees, 140 degrees, 130 degrees, 120 degrees, 110 degrees, 100 degrees, 90 degrees, 80 degrees, 70 degrees, 60 degrees, 50 degrees, 40 degrees, 30 degrees, 20 degrees, 10 degrees, 5 degrees, or less. In a preferred example, the direction of flow of blood through the treatment/stabilization unit can be substantially orthogonal to the direction of flow of blood through the blood input area.

The cartridge assembly can be configured to separate a variety of analytes from the blood sample. For example, the treatment/stabilization unit can be configured to separate out cells, plasma, serum, lipids, platelets, specific cell types, DNA (e.g., tumor cfDNA), RNA, protein, inorganic materials, drugs, or any other components. In specific embodiments, the treatment/stabilization unit can be configured to separate out total cholesterol, HDL cholesterol, LDL cholesterol, triglycerides, Creatinine, Alanine Aminotransferase, and glucose from the blood sample.

The cartridge assembly can be configured to be operated at an angle that is substantially orthogonal to the ground. For example, the cartridge assembly can be configured to receive blood from a sample acquisition device that is attached to a patient's arm and lie substantially parallel to the patient's arm. The cartridge assembly can also be configured to operate at any angle to the ground. The cartridge assembly can be operated at an angle substantially parallel to the ground, or at an angle of about 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, or about 180 degrees to the ground.

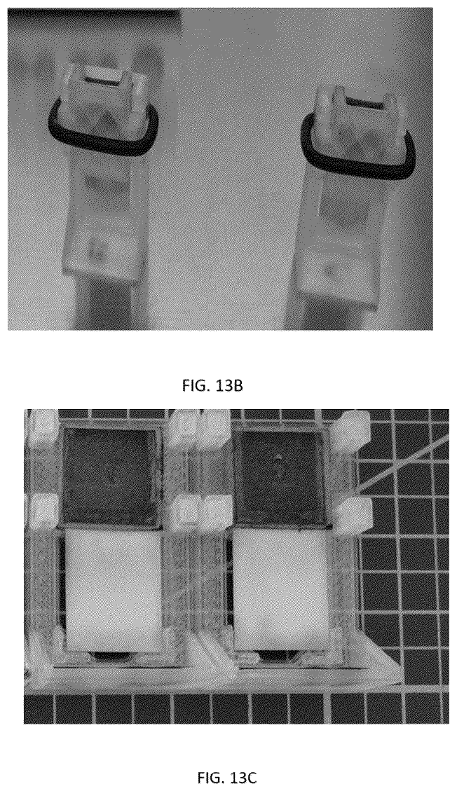

FIGS. 13-18 illustrate embodiments of a blood separation assembly. The components of these embodiments can be configured for use in any other embodiments described herein. This can include modifying and/or reducing the form factor of the several components for use in other embodiments. For example, the components of FIGS. 13-18 can be configured for use in the embodiment of FIG. 12.

The assembly 1300 of FIG. 13A can be comprised of several components. For example, a first assembly structure 1310, a second assembly structure 1330, and a treatment/stabilization unit 1320 which can comprise a pre-filter 1322, a separation membrane 1324, and a collection matrix 1326. The first assembly structure 1310 and second assembly structure 1330 can be configured in a way to keep the components of the treatment/stabilization unit 1320 in a substantially vertical orientation. This allows the fluid sample 1350 to flow in a direction substantially parallel to the longitudinal axis 1360 of the blood separation assembly and encourage the flow of the fluid sample 1350 through and along the treatment/stabilization unit with the aid of wicking forces of gravity. The first assembly structure 1310 and second assembly structure 1330 can be configured so that the first assembly structure 1310 can slide and lock into the second assembly structure 1330. Once in a locked position, as shown in the furthest left image of FIG. 13A, the first assembly structure 1310 can be constrained in one or more degrees of freedom. For example, the first assembly structure 1310 may only be moveable in a direction away from the second assembly structure 1330. The first assembly structure 1310 can be taken in and out of the locked position to allow access to the components of the treatment/stabilization unit 1320 sandwiched between the first assembly structure 1310 and second assembly structure 1330.

The collection matrix 1326 can be configured to be larger than both the separation membrane 1324 and the pre-filter 1322. For example, the collection matrix can be about 10%, about 20%, about 30%, about 40%, about 50%, about 60%, about 70%, about 80%, about 90%, about 100%, about 110%, about 120%, about 130%, about 140% or about 150% or more longer than the length of the separation membrane and pre-filter. In an assembled configuration, as shown in the furthest left image of FIG. 13A, a bottom piece of the collection matrix can be exposed. For example, the exposed portion of the collection matrix can be about 5%, about 10%, about 15%, about 20%, about 25%, about 30%, about 35%, about 40%, about 45%, about 50%, about 55%, about 60%, about 65%, about 70%, or about 75% or more of the length of the collection matrix. The exposed portion of the collection matrix allows easier access to the sample collected on the collection matrix after a fluid sample has passed through the blood separation assembly. For example, the exposed portion can be cut away from the rest of the collection matrix without the need to separate the collection matrix from the other components of the treatment/stabilization unit. The exposed portion can also be separated from the rest of the collection matrix with the use of a perforated line. This can allow the exposed portion to be pulled away from the rest of the collection matrix without negatively affecting the viability and/or performance of the collection matrix itself.

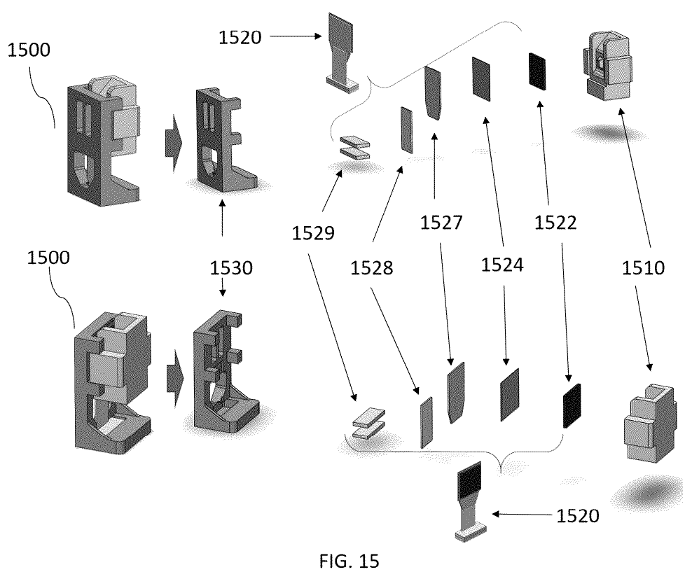

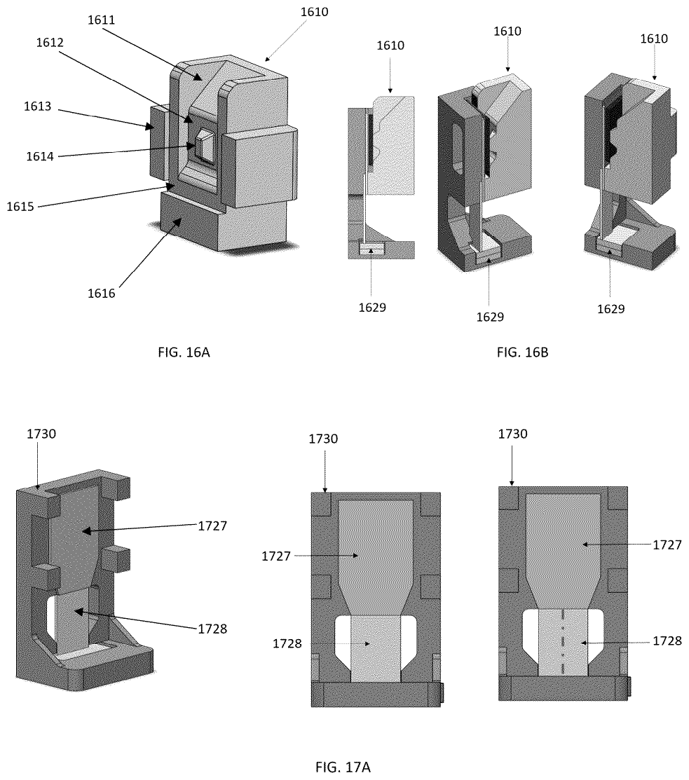

FIG. 15 illustrates perspective views of the several components of the blood separation assembly 1500. The treatment/stabilization unit 1520 can be comprised of several components. For example, the treatment/stabilization unit can comprise a pre-filter 1522, a separation membrane 1524, and a multi-piece collection matrix including a top piece 1527 and a bottom piece 1528. The base of the bottom piece 1528 of the multi-piece collection matrix can be configured to abut absorbent pads 1529. The pre-filter 1522 of the treatment/stabilization unit can be disposed adjacent to the first assembly structure 1510 of the blood separation assembly 1500. The separation membrane can be disposed adjacent to the pre-filter 1522. The multi-piece collection matrix can be disposed adjacent to the separation membrane 1524 and the second assembly structure 1530. The bottom piece of the multi-piece collection matrix can be exposed while the blood separation assembly is in an assembled configuration. The bottom piece of the multi-piece collection matrix can be separated from the top piece by cutting the bottom piece away from the top piece. The top and bottom piece of the multi-piece collection matrix can also be separated by a perforated line, as explained above in FIG. 13, allowing the bottom piece to be pulled away from the top piece. The top and bottom piece of the multi-piece collection matrix can be configured such that an overlap exists between the top and bottom piece. For example, the top and bottom piece of the multi-piece collection matrix can overlap by about 1 mm, 2 mm, 3 mm, 4 mm, or 5 mm or more.

By only extracting the exposed bottom piece of the multi-piece collection matrix, many benefits can be observed in fluid sample analysis. For example, if the multi-piece collection matrix absorbed a blood sample from a subject, extracting only the exposed bottom piece of the multi-piece collection matrix can lead to lower hemolysis levels and a higher analyte yield per surface area. The lower hemolysis in the exposed bottom piece of the multi-piece collection matrix may be because this portion of the multi-piece collection matrix is less constrained, and therefore, cells in this area are less prone to bursting. The exposed bottom piece of the multi-piece collection matrix can allow up to about a 10%, a 20%, a 30%, a 40%, a 50%, a 60%, a 70%, a 80% or a 90% or higher reduction in hemolysis as compared to the unexposed top piece of the multi-piece collection matrix. The exposed bottom piece of the multi-piece collection matrix can allow up to about a 10%, a 20%, a 30%, a 40%, a 50%, a 60%, a 70%, a 80% or a 90% or higher increase in analyte yield per surface area as compared to the unexposed top piece of the multi-piece collection matrix.

FIG. 16A illustrates a perspective view of a first assembly structure which can be configured to provide structural support to the treatment/stabilization unit and blood separation assembly. Additionally, the first assembly can be configured to provide a containment mechanism for incoming sample and to direct the sample onto the desired surface such as the pre-filter and preventing it from directly accessing other surfaces such as the matrix. The first and second assembly structures can be configured to hold the treatment/stabilization unit in an orientation where the planar surfaces of the components in the treatment/stabilization unit are substantially orthogonal to the ground. The planar surfaces of the components in the treatment/stabilization unit can also be substantially parallel to the ground in some embodiments. The first assembly structure may be configured to include a blood input area 1611 which can receive a blood sample from a subject. The blood input area 1611 may be sized and shaped to impact and/or control the volume of sample entering the blood separation assembly. The blood separation assembly can also be configured to have a full perimeter seal by configuring the blood input area to be an inlet channel and not of an open funnel design. The blood sample from the subject can enter the blood input area and accumulate in a recess 1612 of the first assembly structure 1610. The first assembly structure can also be configured to include several structural components. For example, it may include a first compression region 1614, a second compression region 1615, a third compression region 1616, and compression stops 1613 which can rest on one side, or both sides of the first assembly structure.

The first compression region 1614 can be configured to provide a source of pressure to a central area of the treatment/stabilization unit. The second compression region 1615 can be configured to provide a source of pressure to a lower region of the treatment/stabilization unit. The third compression region 1616 can be configured to provide a source of pressure to the bottom piece of the multi-piece collection matrix. The compression stops 1613 can be configured in a way to ensure the first, second, and third compression regions to do not over compress the treatment/stabilization unit.

The compression force applied by the first, second, and third compression regions can be configured to ensure good contact between components of the treatment/stabilization unit to allow for optimized blood flow through the treatment/stabilization unit. The contact created between the components of the treatment/stabilization unit by the compression force can be sufficient to achieve the wicking forces required for the blood sample to flow across the treatment/stabilization unit. This compression force can be enough to encourage an optimal flow of blood through the treatment/stabilization unit without compromising, deforming, or otherwise damaging the materials of the several components of the treatment/stabilization unit. The compression force applied to the treatment/stabilization unit may be about 20 pounds, 19 pounds, 18 pounds, 17 pounds, 16 pounds, 15 pounds, 14 pounds, 13 pounds, 12 pounds, 11 pounds, 10 pounds, 9 pounds, 8 pounds, 7 pounds, 6 pounds, 5 pounds, 4 pounds, 3 pounds, 2 pounds, or 1 pound or less. The compressed thickness of the treatment/stabilization unit can be about 10%, about 20%, about 30%, about 40%, about 50%, about 60%, about 70%, about 80%, or about 90% or more of the uncompressed thickness of the treatment/stabilization unit. The compressed thickness of the treatment/stabilization unit can be about 2.0 mm, 1.75 mm, 1.5 mm, 1.25 mm, 1.0 mm, 0.75 mm, 0.50 mm, or 0.25 mm or less.

The first assembly structure and the second assembly structure can be held/clamped together with any suitable coupling mechanism. Examples of the coupling mechanisms can include, but are not limited to, male-to-female fasteners (e.g., mating or interlocking fasteners, hooks and holes, hooks and loops such as Velcro™, a female nut threaded onto a male bolt, a male protrusion inserted into a female indentation, a male threaded pipe fitted into a female threaded elbow in plumbing, a male universal serial bus (USB) plug inserted into a female USB socket, etc.), tethers (e.g., string tethers), adhesives (e.g., solids, semi-solids, gels, viscous liquids, etc.), magnets (e.g., electromagnet or permanent magnet), and other grasping mechanisms (e.g., one or more robotic arms). In an example, the coupling can be performed using an electric field between the inlet port and the sample acquisition device. Coupling mechanisms can further include clamps, springs, screws, elastomer bands, or other stretchable component which can reach around the first and second assembly structures and hold them together. In other embodiments, the first assembly structure and second assembly structure can be held together via groves configured in the bodies of the two structures. The coupling mechanisms holding the two structures together can be configured to achieve the desired compression force or a desired compression distance between the components of the treatment/stabilization unit. The coupling mechanisms can be configured to apply an even force across an entire surface area of the treatment/stabilization unit. The coupling mechanism can also be configured to apply different forces to different areas of the treatment/stabilization unit.

The compression stops 1613 can be configured to ensure that the coupling mechanism holding the two structures together meet, and do not surpass, the desired compression force or compression distance. The compression stops can also comprise a sensor which measures the compression force applied to the treatment/stabilization unit and alert a user if the force applied to the treatment/stabilization unit exceeds a maximum applied force. The thickness of the compression stops can be configured to be the same thickness as the first assembly structure. For example, the thickness of the compression stops may be about 0.090, 0.080, 0.070, 0.060, 0.050, 0.040, 0.030, 0.020, or about 0.010 inches or less. The thickness of the compression stops can also be configured to be less than or greater than the thickness of the first assembly structure. For example, the thickness of the compression stops can be about 50%, about 60%, about 70%, about 80%, about 90%, about 100%, about 110%, about 120%, about 130%, about 140%, or about 150% or more of the thickness of the first assembly structure.

The compression stops can be fabricated from materials such as polypropylene, polyvinyl chloride, polyvinylidene chloride, low density polyethylene, linear low density polyethylene, polyisobutene, poly[ethylene-vinylacetate]copolymer, lightweight aluminum foil and combinations thereof, stainless steel alloys, commercially pure titanium, titanium alloys, silver alloys, copper alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, partially resorbable materials, such as, for example, composites of metals and calcium-housing based ceramics, composites of PEEK and calcium housing based ceramics, composites of PEEK with resorbable polymers, totally resorbable materials, such as, for example, calcium housing based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

In other embodiments the first assembly structure and second assembly structure of the blood separation assembly can be configured to be one single piece. This may be accomplished by the use of a living hinge, or other similar technique, allowing the single piece to be flexible. In other embodiments, the blood separation assembly can comprise more than two pieces. The addition of more pieces to the blood separation assembly can be configured to improve the functionality, moldability, and/or the manufacturability of the blood separation assembly.

The blood separation assembly can also be configured to include the addition of additional recesses which can be configured to adjust the air exposure to the collection matrix. This can aid in the control of blood plasma concentration and the rate of desiccation. The blood plasma concentration can be about 1.0, 0.9, 0.8, 0.7, 0.6, 0.5, 0.4, 0.3, 0.2, or 0.1 μL/mm². The desiccation of the blood sample can occur in less than about 24, 23, 22, 21, 20, 19, 18, 17, 16, 15, 14, 13, 12, 11, 10, 9, 8, 7, 6, 5, 4, 3, 2, or 1 hours. The additional recesses may be of any size and shape as long as they do not impact the structural performance of the other components of the blood separation assembly. The number of additional recesses may be chosen to achieve a desired effect on temperature and humidity which may affect the rate of desiccation. The additional recesses can allow for air inside the blood separation assembly to be displaced and for the pressure inside the blood separation assembly to equalize with the pressure conditions that exist external to the blood separation assembly. The number of additional recesses may be limited if the pressure conditions inside the blood separation assembly are desired to be different than the pressure conditions external to the blood separation assembly. For example, a desired pressure differential between the internal components and external environment can encourage better blood flow through the treatment/stabilization unit without leading to excess hemolysis of the blood sample.

The blood separation assembly can be 3D printed, injection molded, or machined. The blood separation assembly can include or can be fabricated from materials such as polypropylene, polycarbonate, or other similar materials which do not interfere with or alter the properties of the sample passing through the treatment/stabilization unit.

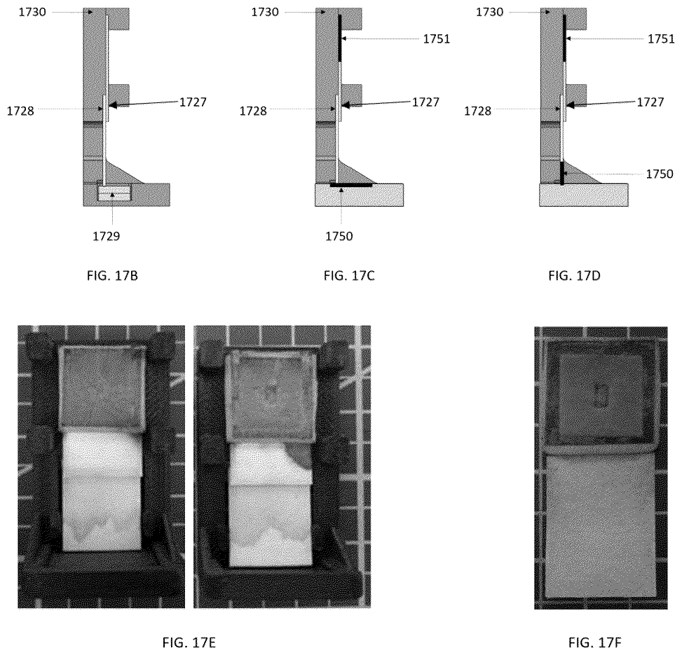

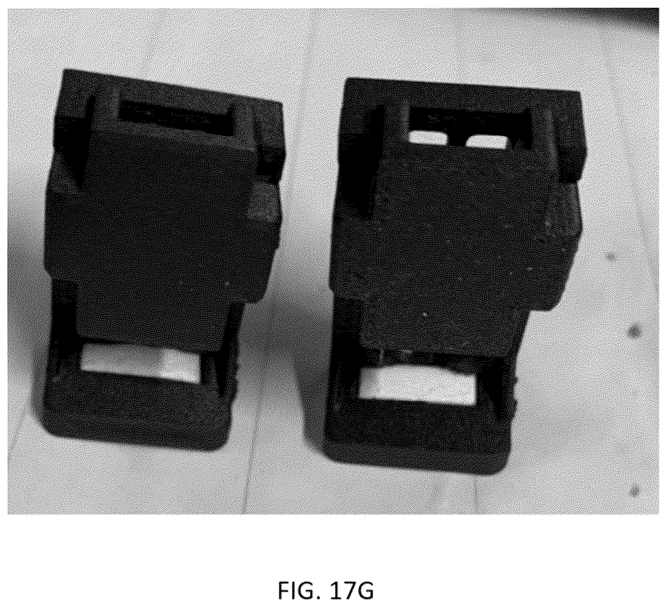

FIG. 17A illustrates an exemplary multi-piece collection matrix comprising a top piece 1727 and a bottom piece 1728 disposed adjacent to a second assembly structure 1730. The multi-piece collection matrix can be configured to include two or more pieces. For example, the multi-piece collection can be configured to include two, three, four, five, or more pieces.

The multi-piece collection matrix can have a volume sufficient to collect a desired amount of the product (e.g., serum or plasm) on the separation membrane. The multi-piece collection matrix can be configured to hold (or contain) at least about 1 μL, 5 μL, 10 μL, 20 μL, 30 μL, 40 μL, 50 μL, 60 μL, 70 μL, 80 μL, 90 μL, 100 μL, 110 μL, 120 μL, 130 μL, 140 μL, 150 μL, 200 μL, 300 μL, 400 μL, 500 μL, 600 μL, 700 μL, 800 μL, 900 μL, 1,000 μL, or more of the product of the separation membrane. The multi-piece collection matrix can be configured to hold (or contain) at most about 1,000 μL, 900 μL, 800 μL, 700 μL, 600 μL, 500 μL, 400 μL, 300 μL, 200 μL, 100 μL, 50 μL, 10 μL, 1 μL, or less of the product of the separation membrane.

The top piece 1727 of the multi-piece collection matrix can be configured so that the entire planar surface area of the top piece is covered by the separation membrane in an assembled blood separation assembly. The bottom piece of the multi-piece collection matrix can be configured to be exposed and untouched by the separation membrane or pre-filter in a blood separation assembly. The bottom piece of the multi-piece collection matrix can be configured to be exposed in order to improve sample analysis. The exposed bottom piece of the multi-piece collection matrix may have a surface area of about 50, 60, 70, 80, 90, 100, 110, 120, 130, 140, 150, 160, 170, 180, 190, 200, 210, 220, 230, 240, or 250 or more mm². The exposed bottom piece can be configured to simply pull away from the top piece of the collection matrix, preventing the need to cut, tear, or otherwise bifurcate the treatment/stabilization unit. For example, through the use of a perforated line separating the top piece and bottom piece. As shown in FIG. 17A, the bottom piece of the multi-piece collection matrix can be further divided into multiple segments. The bottom piece can be divided along a longitudinal axis, as shown in FIG. 17A, and can also be divided along a horizontal axis. The bottom piece can also be divided along both a longitudinal and horizontal axis.

The top piece and bottom piece of the multi-piece collection matrix can be configured such that each piece can be of a different geometry, material, thickness, coating, and chemistry. The top piece can be configured such that the top piece funnels the blood sample from a wider portion of the multi-piece collection matrix to a narrower portion of the multi-piece collection matrix. The bottom piece may be configured to have a geometry optimized for a sample collection elution method as will be discussed herein. The sample may also be analyzed from the top piece of the multi-piece collection matrix. The top piece and bottom piece of the multi-piece collection matrix may be configured such that the top piece and bottom piece are of different thicknesses. The top piece may be thicker than the bottom piece, the bottom piece may be thicker than the top piece, or the top and bottom piece may be of the same thickness. The thickness of the multi-piece collection matrix can be configured to allow the multi-piece collection matrix to hold (or contain) a specified amount of volume of liquid. The blood separation assembly may also be configured to have multiple collection matrices. The multiple collection matrices can be configured to be multi-piece collection matrices, single piece collection matrices, or a combination of the two.

FIG. 17B illustrates a side sectional view of a multi-piece collection matrix disposed adjacent to a second assembly piece 1730 with absorbent pads 1729 configured to rest at the base of the bottom piece 1728 of the multi-piece collection matrix. As illustrated in FIG. 17B, the bottom piece and the top piece of the multi-piece collection matrix can be configured such that the two pieces overlap with each other. The two pieces can also be configured such that there is no overlap between the two pieces. For example, the top piece and the bottom piece can be separated by a perforated strip allowing the bottom piece to easily be separated from the top piece.

The absorbent pads 1729 enable metering of a blood sample collected in the blood separation assembly. The absorbent pads can be configured to collect any excess separated blood sample or liquid beyond the saturation volume of the multi-piece collection matrix. There can be one absorbent pad or multiple absorbent pads. If multiple absorbent pads are used, they may be configured such that they are stacked on top of each other or aligned end to end. The absorbent pads may be thicker, or they may be thinner than the multi-piece collection matrix.

The absorbent pads can be configured to be directly integrated with the multi-piece collection matrix, or the absorbent pads can be separated from the multi-piece collection matrix. If the absorbent pads are configured to be directly integrated with the multi-piece collection matrix, the absorbent pads can be a portion of the bottom piece of the multi-piece collection matrix that is cut off, or that can be separated from the bottom piece of the multi-piece collection matrix by a perforated strip. If the absorbent pads are configured to be separate from the multi-piece collection matrix, they can be configured such that sufficient contact between the absorbent pads and the bottom piece of the multi-piece collection matrix is achieved. This can be done by adding an additional component below the absorbent pads which allows the absorbent pads to remain in contact with the bottom piece of the multi-piece collection matrix. The absorbent pads can be configured to be in contact with the planar surface of the bottom piece of the multi-piece collection matrix on one side or both sides of the bottom piece.

The absorbent pads can be configured to change in size and geometry in order to adjust the volume of blood sample or liquid the absorbent pads are desired to hold (or contain). Absorbent pads can also be configured to rest in other areas of the blood separation assembly where a blood sample or other liquid may leak and need to be collected.

The incorporation of absorbent pads in the cartridge assembly that hold any excess liquid that cannot be contained by the collection matrix allows the volume of liquid collected from a patient to be greater than the volume the collection matrix can hold. For example, if the saturation point of the collection matrix is 50 μL and the absorbent capacity of the absorbent pads is 300 μL this allows for a variety of scenarios to occur. For example, introducing 50 μL of sample to the treatment/stabilization unit will result in approximately 50 μL of sample to be collected in the collection matrix and approximately 0 μL to be held in the absorbent pads. Introducing 75 μL of sample to the treatment/stabilization unit will result in approximately 50 μL of sample to be collected in the collection matrix and approximately 25 μL to be held in the absorbent pads. So, a maximum input volume of sample liquid to the cartridge assembly will be the capacity of the collection matrix in addition to the capacity of the absorbent pads. The capacity of the collection matrix and capacity of the absorbent pads can be adjusted in order to change a desired amount and/or the maximum amount of sample liquid to be obtained from a patient. A cartridge assembly comprising absorbent pads can be configured to perform in this fashion because different blood samples from different patients can have different levels of hematocrit, meaning one volume of blood collected from one patient will provide a greater or lower volume of plasma than the same amount of volume of blood collected from a second patient. The absorbent pads help when it may not be feasible to measure a precise amount of blood to enter the cartridge assembly and a user may not have to worry about overfilling the cartridge assembly. For example, without absorbent pads, obtaining a high hematocrit blood sample could result in the collection matrix not receiving enough plasma. On the other hand, without absorbent pads, obtaining a low hematocrit blood sample could results in the oversaturation of the collection matrix.

As illustrated in FIGS. 17B and 17C, in alternative embodiments, the absorbent pads 1729 can instead be absorbent paper 1750. Non-limiting examples of absorbent paper may include fibrous paper with high absorbent capacities such as 31-ETF, CF-12, CF-9, or the like. As illustrated in FIG. 17B, the absorbent paper 1750 can be configured to rest perpendicular at the base bottom of the collection matrix. The absorbent paper can be folded or otherwise manipulated to create different geometries and to create a spring-like action to keep the paper in contact with the matrix. Alternatively, as illustrated in FIG. 17C, the absorbent paper 1750 can be parallel to the collection matrix. If the absorbent paper 1750 is parallel to the collection matrix, the absorbent paper may rest flush to the base of the collection matrix or overlap with the base of the collection matrix. For example, the absorbent paper may have 0 mm, 1 mm, 2 mm, 3 mm, 4 mm, or 5 mm of overlap with the collection matrix. The absorbent paper 1750 can be configured to pull away from the bottom piece of the collection matrix by use of a perforated strip separating the two components.

In addition to the absorbent paper 1750 of FIGS. 17B and 17C, an additional hydrophilic layer 1751 can be configured to rest at the top of the collection matrix. The hydrophilic layer 1751 can be configured to be parallel to the collection matrix. If the hydrophilic layer 1751 is parallel to the collection matrix, the hydrophilic layer 1751 can rest flush with the top of the collection matrix, or overlap with the top of the collection matrix. For example, the hydrophilic layer may have 0 mm, 1 mm, 2 mm, 3 mm, 4 mm, or 5 mm of overlap with the collection matrix. The hydrophilic layer 1751 can be configured to pull away from the bottom piece of the collection matrix by use of a perforated strip separating the two components. The use of the absorbent paper and/or the hydrophilic layer can be for the same purpose, to absorb excess sample, as the use of the absorbent pads as described above.

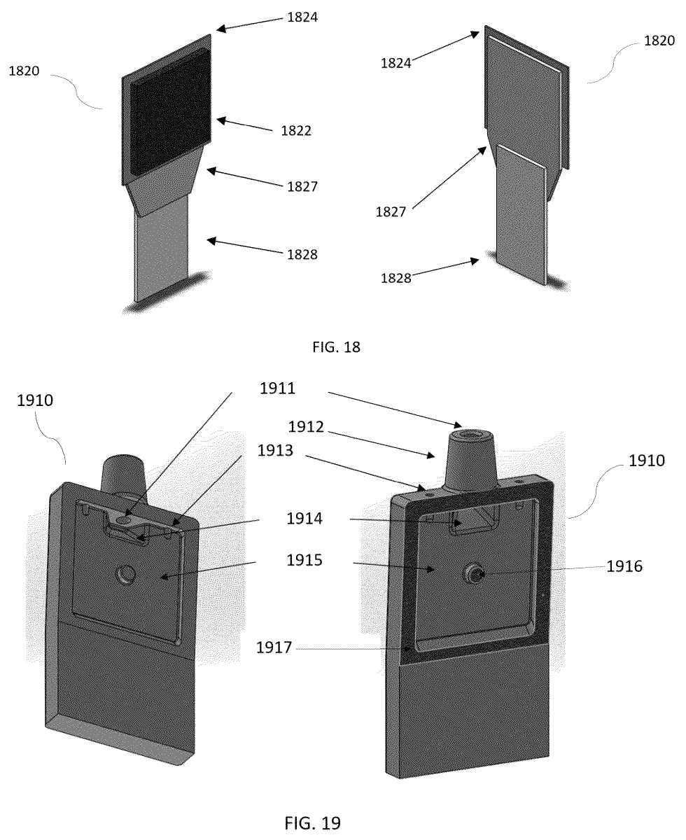

FIG. 18 illustrates perspectives views of the treatment/stabilization unit 1820 that can be used in embodiments described herein. The pre-filter 1822 can be configured to be a first component a blood sample or other liquid comes in contact with in the blood separation assembly. The pre-filter 1822 can be configured to comprise a smaller surface area, as illustrated in FIG. 18, than the separation membrane 1824 disposed immediately after and adjacent to the pre-filter. The pre-filter can also be configured to comprise a surface area the same as or larger than the separation membrane. The pre-filter can be configured to separate or otherwise filter out certain components of the liquid or blood sample prior to the liquid or blood sample reaching the other components of the treatment/stabilization unit. The pre-filter can be coarser than the other components of the treatment/stabilization unit and can allow the overall throughput of to increase. For example, the treatment/stabilization unit can separate 300 μL of a sample with a pre-filter whereas the treatment/stabilization unit could only separate 100 μL of a sample without the pre-filter.

The separation membrane 1824 can be configured to separate and contain cellular components of a blood sample while allowing the plasma/serum of the blood to be collected by the collection matrix disposed immediately after and adjacent to the separation membrane. For example, the separation membrane can be of a Leukosorb® material. The surface area of the separation membrane can be configured to be larger than the surface area of the other components in the treatment/stabilization unit to ensure no blood sample bypasses the separation membrane before reaching the collection matrix.

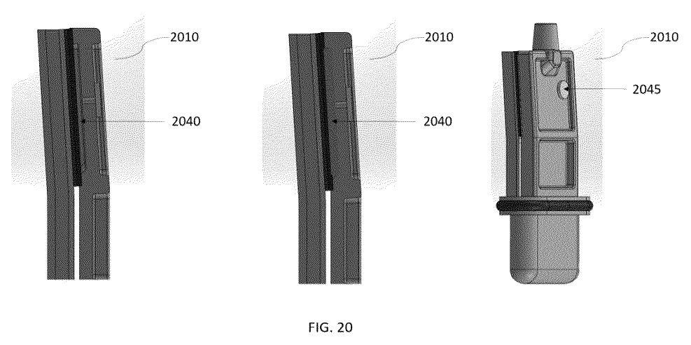

FIGS. 19 and 20 illustrate cartridges and cartridge assemblies. The components of these embodiments can be configured for use in any other embodiments described herein. This can include modifying and/or reducing the form factor of the several components for use in other embodiments. For example, the components of FIGS. 19-20 can be configured for use in the embodiment of FIG. 12.

FIG. 19 illustrates perspective views of an example cartridge 1910 that can be configured for use in a cartridge assembly implementing a treatment/stabilization unit and configured to collect liquid or liquid-like samples (e.g., liquid blood) as described herein. The cartridge 1910 can comprise a coupling unit 1912 that can be configured to couple (e.g., releasably or permanently couple) to a sample acquisition device (e.g., a port in a cartridge chamber of any of the same acquisition devices disclosed herein) using any of the coupling mechanisms described herein. For example, the coupling unit 1912 can have a luer type fitting to mate with the cartridge chamber port of the sample acquisition device. The coupling unit 1912 can comprise an opening, an inlet 1911, or a channel that is configured to serve as a pathway for the blood to flow from the sample acquisition device and towards the cartridge assembly (e.g., into the cartridge assembly). For example, the inlet 1911 can receive the blood from the sample acquisition device and direct the flow of blood through the funnel 1914 and into the recess 1915 that allows blood to accumulate in a space adjacent to the surface of the pre-filter of a treatment/stabilization unit. The cartridge 1910 can also be configured to include a first compression area 1916. The cartridge may further comprise a second compression area 1917 which can act to seal the entire perimeter of the treatment/stabilization unit. Configuring the cartridge 1910 in this manner prevents the flow of blood from being able to bypass the pre-filter and separation membrane of the treatment/stabilization unit. For example, in a situation where blood is being introduced into the recess 1915 through the inlet 1911 faster than the treatment/stabilization unit can process the blood. The cartridge 1910 can also be configured to include vents 1913 that can allow for pressure equalization between the recess 1915 and the environments external to the cartridge 1910. This may allow air to be displaced and/or a vacuum or other pressure conditions that exist external to the cartridge to equalize within the recess 1915, through the inlet 1911 and into the upstream portion of the blood acquisition device. The vents 1913 can reduce or eliminate completely a pressure differential across the treatment/stabilization unit directly. In some examples, the vents may be eliminated to allow for a pressure differential to occur across the treatment/stabilization unit to encourage the flow of blood through the components of the treatment/stabilization unit. The cartridge can be completely opaque, or fully or partially transparent to allow a user to observe the accumulation of blood in the recess 1915 during a blood draw. Visualization of the accumulation of blood in the recess 1915 can be used as an indication that the treatment/stabilization unit has processed as much blood as possible and that the draw may be stopped.

The implementation of a blood separation assembly into a cartridge assembly as described herein allows for a method to perform a blood draw with an entire system comprising components of the treatment/stabilization unit that have planar surfaces that are substantially orthogonal to the ground. A method such as this is desirable in a process, for example, where the sample acquisition device is configured to collect blood while attached to a patient's arm. This further allows a for a low-profile design of a sample acquisition device.

FIG. 20 illustrates side sectional views and a perspective view of a cartridge assembly 2010 that can be configured to include a visual metering element to indicate to a user when a sufficient amount of blood from a patient has been received in the cartridge assembly 2010. The cartridge assembly could be configured to use a pre-metering chamber. The pre-metering chamber can be configured to provide a visual indication to a user of when a correct amount of blood has been collected by visually confirming the chamber has been filled. When a correct amount of blood has been filled, a user will be able to visually see that the pre-metering chamber has been filled. The pre-metering chamber can be configured to include a semi-permeable membrane which enables air to escape but not blood or other liquids so that air can be displaced as the entire chamber is filled with blood. Once filled with blood, the blood can be advanced to the treatment/stabilization unit manually via a piston configuration or diaphragm where check valves may be implemented to prevent a backflow. In another example, blood can advance automatically when the seal is broken at the end of the draw between the skin of a patient and the sample collection device. When this occurs, a large pressure differential is created across the inlet which can be used to advance the blood sample or trigger the advancement of the blood sample.

In another embodiment, a system can be used in which the properties of the collection matrix cause it to shut off when a maximum volume of blood has been processed. Once the collection matrix absorbs a maximum volume of blood from the patient, blood will stop being processed and begin to accumulate in the recess upstream. This accumulation can be configured to depict to a user a visual indication that enough blood has been collected. For example, in the furthest right image of FIG. 20, the window 2045 can be configured to turn from white to red when blood reaches it after accumulation in the recess has occurred. Alternatively, the indicator could comprise an absorbent material that absorbs blood and changes color. Alternatively, the visual indication of blood accumulation in the recess can be seen by a user from the side of the cartridge assembly, as illustrated in the two left images of FIG. 20. The furthest left image illustrates an empty recess 2040, and the middle image in FIG. 20 illustrates a recess 2040 that has been filled with blood after a maximum volume of blood has been contained in the collection matrix.

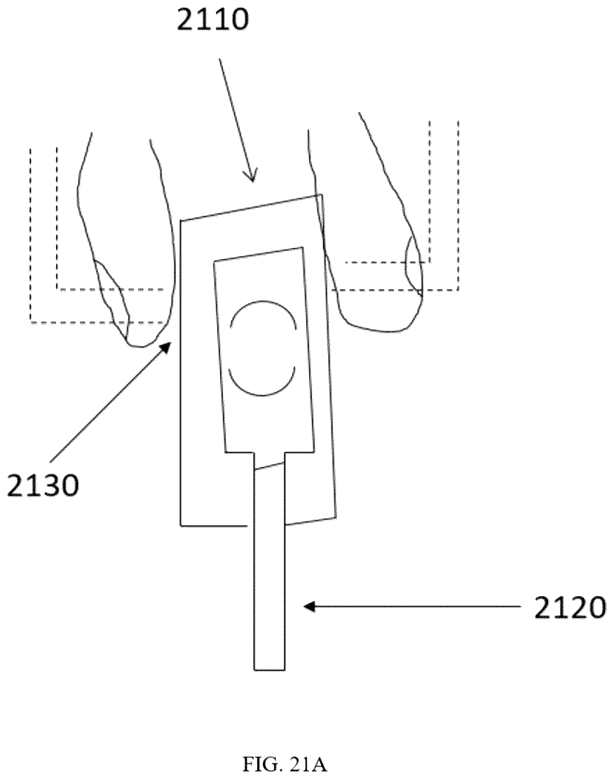

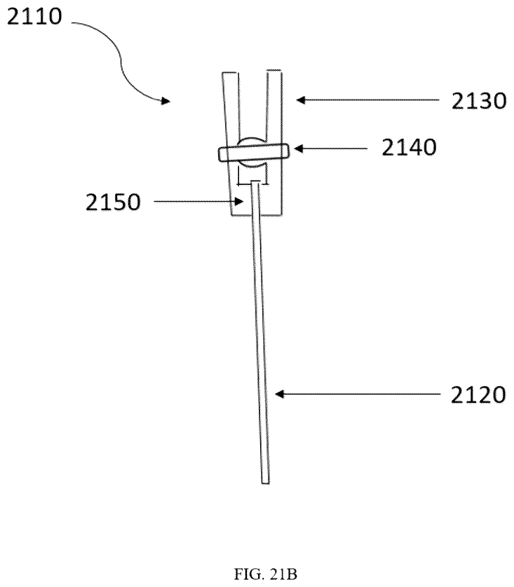

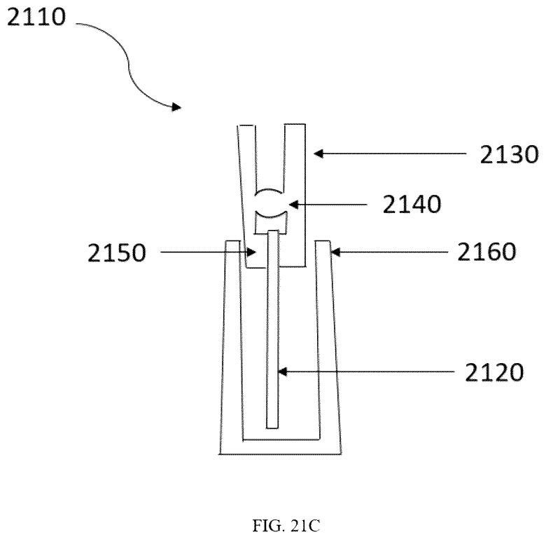

FIGS. 21A-21C illustrate an additional embodiment of a cartridge assembly for the acquisition of a treatment/stabilization unit once a blood separation process has been completed. As illustrated in FIG. 21A, the cartridge can include a releasing mechanism 2110 configured to hold a treatment/stabilization unit 2120 in place. The releasing mechanism 2110 can release the treatment/stabilization unit 2120 upon the application of force at the pressure point 2130. A feature such as this enables the treatment/stabilization unit to be handled in a manual or automated fashion without having to contact the treatment/stabilization unit directly with an additional component (e.g., tweezers, grippers, disposable tips, etc.). This eliminates the possibility of contamination and prevents the need for cleaning or sterilization after the release of the treatment/stabilization unit 2120. The application of force to the pressure point 2130 can be accomplished, for example, by squeezing the pressure point 2130 with fingers. Alternatively, the pressure point 2130 can be highly localized and may only be engaged with the use of grippers (as shown in the dotted lines of FIG. 21A) designed to apply pressure concentrated in a particular area.

FIG. 21B illustrates a releasing mechanism 2110 with the addition of a seal 2140 and grips 2150. As disclosed in other embodiments herein, the seal 2140 may be configured to hermetically seal the cartridge chamber. As the pressure point 2130 of the releasing mechanism 2110 is squeezed, the grips 2150 may release the treatment/stabilization unit 2120. The grips 2150 may be configured to include absorbent pads configured to contact and hold the treatment/stabilization unit 2120 in place. The absorbent pads may be retained on the grips 2150 after the release of the treatment/stabilization unit 2120. The absorbent pads may also be configured to be released from the grips 2150 with, or separately from, the treatment/stabilization unit 2120.

FIG. 21C illustrates a releasing mechanism 2110 with the addition of a guard 2160 which may prevent the inadvertent release of the treatment/stabilization unit 2120. As discussed in embodiments herein, the cartridge may be configured to be releasably coupled to a sample acquisition device or may be inserted into a transport sleeve. The guard 2160 may be configured to be part of the sample acquisition device or transport sleeve, and the treatment/stabilization unit 2120 may only be released once the cartridge is disengaged from the sample acquisition device or transport sleeve. The transport sleeve may be configured to receive the treatment/stabilization unit 2120 upon its release and retain the treatment/stabilization unit 2120 until the treatment/stabilization unit 2120 is ready for testing.

### E. Elution Methods

Further aspects of the present disclosure provide methods of dissociating biomolecule(s), such as, e.g., nucleic acid molecules, proteins, hormones, carbohydrates, lipids, from a collection matrix for further process. Such biomolecules are often derived from a subject, such as a human, and are useful as a biomarker for in vitro diagnostics or for monitoring of a patient's health. Biomarkers may include, for example, alanine aminotransferase (ALT), anti-mullerian hormone (AMH), apolipoprotein A1 (APOA1), apolipoprotein B (APOB), aspartate aminotransferase (AST), blood urea nitrogen (BUN), cadmium (Cd), Chlamydia trachomatis amplified DNA, cholesterol (e.g., HDL, LDL, or total), copper (Cu), cortisol, creatine, dehydroepiandrosterone sulfate (HDEA-S), estradiol (E2), follicle-stimulating hormone (FSH), free thyroxine (fT4), free triiodothyronine (fT3), hemoglobin A1c (HbA1c), hepatitis B antigen, hepatitis C antibody, high-sensitivity C-reactive protein (hs-CRP), HIV-1, HIV-2 antibody and/or antigen, insulin-like growth factor 1 (IGF-1, somatomedin C), insulin, lead (Pb), luteinizing hormone (LH), magnesium (Mg), mercury (Hg), neisseria gonorrhea amplified DNA, progesterone (Pg), prolactin, prostate-specific antigen (PSA), severe acute respiratory syndrome coronavirus 2 antibodies (SAR-CoV-2, immunoglobulin A (IgA), immunoglobulin G (IgG), immunoglobulin M (IgM)), selenium (Se), sex hormone-binding globulin (SHBG), syphilis, testosterone (total), thyroglobulin (TG), thyroid peroxidase antibodies (TPOab), thyroid stimulating hormone (TSH), thyroperoxidase antibody (TPOAb), thyroxine (T4, total), total bilirubin, thrichomonas amplified DNA, triglycerides, thriiodothyronine (T3, total), vitamin B6, vitamin B9 (Folate), vitamin B12, vitamin D (25-OH, D2/25-OH D3), zinc (Zn).

### 1. Mechanical Dissociation

Mechanical dissociation methods can be used to process collection matrices. Such methods can be used to dissociate biomolecules from collection matrices. Non-limiting examples of mechanical dissociation methods include sonication, vortexing, shaking, rocking, nutation, invert-mixing, rotating, soaking, macerating, homogenization, and freeze/thaw cycling.

Collection matrices can be soaked for dissociation of biomolecules. Soaking can be performed in the presence of various buffers or solvents. Alternatively, soaking can be performed with water. Buffers or solvents can include elution buffers, lysis buffers, wash buffers, etc. In some cases, soaking can be performed in the presence of chelators, reducing agents, oxidizing agents, surfactants, protein denaturants, one or more salts, one or more enzymes, or any organic solvents. Soaking can be performed before any other elution methods are performed. Alternatively, soaking can be performed after other methods of elution.

In some cases, the time for soaking collection matrices can be less than 1 minute, less than 5 minutes, less than 10 minutes, less than 20 minutes, less than 30 minutes, less than 50 minutes, less than 60 minutes, less than 2 hours, less than 5 hours, less than 10 hours, less than 20 hours, less than 30 hours, less than 1 days, less than 2 days or less than 3 days. In some cases, the time for soaking may be greater than 1 minute, greater than 5 minutes, greater than 10 minutes, greater than 20 minutes, greater than 30 minutes, greater than 50 minutes, greater than 60 minutes, greater than 2 hours, greater than 5 hours, greater than 10 hours, greater than 20 hours, greater than 30 hours, greater than 1 days, greater than 2 days or greater than 3 days.

Collection matrices can be soaked at a temperature of about 0° C., about 4° C., about 10° C., about 20° C., about 25° C., about 27° C., about 30° C., about 32° C., about 35° C., about 40° C., about 45° C., about 50° C., about 55° C., about 60° C., about 65° C., about 70° C., about 75° C., about 80° C., about 85° C., about 90° C., about 92° C., about 95° C., or about 98° C. In some cases, collection matrices can be soaked at a temperature of less than 0° C., less than 4° C., less than 10° C., less than 20° C., less than 25° C., less than 27° C., less than 30° C., less than 32° C., less than 35° C., less than 40° C., less than 45° C., less than 50° C., less than 55° C., less than 60° C., less than 65° C., less than 70° C., less than 75° C., less than 80° C., less than 85° C., less than 90° C., less than 92° C., less than 95° C., or less than 98° C. In some cases, collection matrices can be soaked at a temperature of more than 0° C., more than 4° C., more than 10° C., more than 20° C., more than 25° C., more than 27° C., more than 30° C., more than 32° C., more than 35° C., more than 40° C., more than 45° C., more than 50° C., more than 55° C., more than 60° C., more than 65° C., more than 70° C., more than 75° C., more than 80° C., more than 85° C., more than 90° C., more than 92° C., more than 95° C., or more than 97° C.

Collection matrices can be processed using sonication. Sonication can be performed before soaking or rehydration. Commercially available instruments can be used for sonication. Sonication can be performed in the presence of buffers, examples of which are presented elsewhere herein. It can be performed at different speeds for different biomolecules. Sonication can be used to lyse cells or shear genomic DNA or proteins. Sonication can be performed with the collection matrix or the soaked collection matrix on ice.

Sonication can be performed in pulses. For instance, sonication can be performed for 10 seconds and then the sample can be rested for 40 seconds. Sonication amplitude can be adjusted according to the target biomolecule in the biological sample. Amplitude used for sonication can be about 1% to about 80%. Amplitude used for sonication can be at least about 1%. Amplitude used for sonication can be less than 5%, less than 10%, less than 15%, less than 20%, less than 25%, less than 30%, less than 50%, less than 60%, less than 70%, or less than 80%. In some cases, amplitude used for sonication can be more than 1%, more than 5%, more than 10%, more than 15%, more than 20%, more than 25%, more than 30%, more than 50%, more than 60%, more than 70%, or more than 80%.

Any of the collection matrices herein can be processed using agitation. Agitation can include vortexing, rocking, mixing, shaking, etc. The speed can be less than 5 rotations per minute (rpm), less than 10 rpm, less than 15 rpm, less than 20 rpm, less than 30 rpm, less than 40 rpm, less than 50 rpm, less than 60 rpm, less than 70 rpm, less than 80 rpm, less than 90 rpm, less than 100 rpm, less than 150 rpm, less than 200 rpm, less than 250 rpm, less than 300 rpm, less than 350 rpm, less than 400 rpm, less than 500 rpm, less than 600 rpm, less than 700 rpm, less than 800 rpm, less than 900 rpm, less than 1,000 rpm, less than 1,500 rpm, less than 2,000 rpm, less than 2,500 rpm, less than 3,000 rpm, less than 3,500 rpm, less than 4,000 rpm, less than 4,500 rpm, less than 5,000 rpm, less than 5,500 rpm, less than 6,000 rpm, less than 6,500 rpm, less than 7,000 rpm, less than 7,500 rpm, less than 8,000 rpm, less than 8,500 rpm, less than 9,000 rpm, less than 9,500 rpm, or less than 10,000 rpm. The speed can be about 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm. The speed can be at least 50 rpm 100 rpm, 200 rpm, 300 rpm, 400 rpm, 500 rpm, 600 rpm, 700 rpm, 800 rpm, 900 rpm, 1000 rpm, 1500 rpm, or 5000 rpm.

Agitation can be performed for at least about 1 second. Agitation can be performed for less than 1 second, less than 5 seconds, less than 10 seconds, less than 15 seconds, less than 20 seconds, less than 30 seconds, less than 50 seconds, less than 60 seconds, less than 80 seconds, less than 100 seconds, less than 120 seconds, less than 5 minutes, less than 10 minutes, less than 20 minutes, less than 30 minutes, less than 50 minutes, less than 60 minutes, less than 50 minutes, less than 60 minutes, less than 2 hours, less than 5 hours, less than 8 hours, less than 10 hours, less than 20 hours, less than 30 hours or less than 50 hours. Agitation can be performed for more than 1 second, more than 5 seconds, more than 10 seconds, more than 15 seconds, more than 20 seconds, more than 30 seconds, more than 50 seconds, more than 60 seconds, more than 80 seconds, more than 100 seconds, or more than 120 seconds, more than 5 minutes, more than 10 minutes, more than 20 minutes, more than 30 minutes, more than 50 minutes, more than 60 minutes, more than 50 minutes, more than 60 minutes, more than 2 hours, more than 5 hours, more than 8 hours, more than 10 hours, more than 20 hours, more than 30 hours, more than 48 hours or more than 50 hours.

Collection matrices can be processed using homogenization. Commercially available homogenizers can be used to process collection matrices. Non-limiting examples include the MACS Octo dissociator, Rotor-Stator homogenizer, bead mills, high pressure homogenizers etc.

Homogenization can be performed at a speed of at least about 500 rpm. Homogenization can be performed at a speed of less than 500 rpm, less than 1,000 rpm, less than 2,000 rpm, less than 4,000 rpm, less than 5,000 rpm, less than 6,000 rpm, less than 8,000 rpm, less than 10,000 rpm, or less than 12,000 rpm. Homogenization can be performed at a speed of more than 100 rpm, more than 500 rpm, more than 1,000 rpm, more than 2,000 rpm, more than 4,000 rpm, more than 5,000 rpm, more than 6,000 rpm, more than 8,000 rpm, more than 10,000 rpm, or more than 12,000 rpm.

Homogenization can be performed at a temperature of at least about 4° C. Homogenization can be performed at a temperature of less than 5° C., less than 10° C., less than 15° C., less than 20° C., less than 25° C., less than 27° C., less than 30° C., less than 32° C., less than 37° C., less than 40° C., less than 42° C., less than 45° C., less than 50° C., less than 55° C., less than 60° C., less than 65° C., less than 70° C., less than 75° C., less than 80° C., less than 85° C., less than 90° C., less than 92° C., less than 95° C., or less than 98° C. Homogenization can be performed at a temperature of more than 4° C., more than 10° C., more than 15° C., more than 20° C., more than 25° C., more than 27° C., more than 30° C., more than 32° C., more than 37° C., more than 40° C., more than 42° C., more than 45° C., more than 50° C., more than 55° C., more than 60° C., more than 65° C., more than 70° C., more than 75° C., more than 80° C., more than 85° C., more than 90° C., more than 92° C., more than 95° C., or more than 97° C.

### 2. Enzymatic Digestion

Collection matrices can be processed using enzymatic dissociation. Enzymatic dissociation can be performed with proteases, carbohydrate digesting molecules, nucleases, lipases, etc. One or more enzymatic dissociation methods can be used for the same collection matrix. For example, a collection matrix can be treated with a protease and a nuclease at the same time. Enzymes used can be naturally occurring or synthetic. They can be isolated from recombinant cells.

Proteases can be used for enzymatic dissociation. Non-limiting examples of proteases include trypsin, Proteinase K, pepsin, chymotrypsin, papain, bromelain, subtilisin, or elastase. A protease can be a serine protease, a cysteine protease, a threonine protease, an aspartic protease, a glutamic protease, or a metalloprotease, or an asparagine peptide lyase. Proteases can be used to dissociate a target protein. Alternatively, proteases can be used to dissociate proteins for the isolation of other biomolecules such as nucleic acids. For example, proteases can be used to disentangle nucleic acids from chromatin.

Protease digestion can be performed in the presence of buffers or solvents. Buffers used can be commercially available buffers. Buffers can comprise EDTA, EGTA, citrate, sodium chloride, LiCl, potassium phosphate, ammonium sulfate, ammonium chloride, magnesium chloride, magnesium sulfate, Tris-HCl, MOPS, HEPES, MES, Dithiothreitol (DTT), (3-mercaptoethanol, TECP, (SDS), guanidine hydrochloride, Guanidinium thiocyanate (GITC), Urea, glutathione (GSH), glutathione disulfide (GSSG), NADPH, ascorbic acid, retinoic acid, and tocopherols or other salts and organic solvents.

Protease digestion can be performed for about 10 minutes. Protease digestion can be performed for less than 10 minutes, less than 15 minutes, less than 30 minutes, less than 50 minutes, or less than 60 minutes. Protease digestion can be performed for less than 1 hour, less than 2 hours, less than 3 hours, less than 4 hours, less than 5 hours, less than 8 hours, less than 10 hours, less than 12 hours, less than 14 hours, less than 16 hours, less than 18 hours, or less than 24 hours. Protease digestion can be performed for more than 10 minutes, more than 15 minutes, more than 30 minutes, more than 50 minutes, or more than 60 minutes. Protease digestion can be performed for more than 1 hour to more than 18 hours. Protease digestion can be performed for more than 1 hour, more than 2 hours, more than 3 hours, more than 4 hours, more than 5 hours, more than 8 hours, more than 10 hours, more than 12 hours, more than 14 hours, more than 16 hours, more than 18 hours, or more than 24 hours.

Enzymes can be used for carbohydrate digestion. Enzymatic digestion of carbohydrates can be performed to degrade a polysaccharide coating on the collection matrix. Alternatively, it can be performed to digest a target biomolecule or a biological sample such as a cell. Examples of such enzymes include but are not limited to: Macerozyme R-10, pectinase, hemicellulase, amylase, xylanase, cellulase, sucrose, maltase etc.

Buffers used for carbohydrate digestion can be commercially available. Buffers can include sodium phosphate, sodium chloride, sodium hydroxide, ethylene glycol, sodium acetate buffer, EDTA, EGTA, citrate, sodium chloride, LiCl, potassium phosphate, ammonium sulfate, ammonium chloride, magnesium chloride, magnesium sulfate, Tris-HCl, MOPS, HEPES, MES Dithiothreitol (DTT), β-mercaptoethanol, TECP, glutathione (GSH), glutathione disulfide (GSSG), NADPH, ascorbic acid, retinoic acid, and tocopherols or other salts or organic solvents.

Carbohydrate digestion can be performed for about 10 minutes. Carbohydrate digestion can be performed for less than 10 minutes, less than 15 minutes, less than 30 minutes, less than 50 minutes, or less than 60 minutes. Carbohydrate digestion can be performed for less than 1 hour, less than 2 hours, less than 3 hours, less than 4 hours, less than 5 hours, less than 8 hours, less than 10 hours, less than 12 hours, less than 14 hours, less than 16 hours, or less than 18 hours. Carbohydrate digestion can be performed for more than 10 minutes, more than 15 minutes, more than 30 minutes, more than 50 minutes, or more than 60 minutes. Carbohydrate digestion can be performed for more than 1 hour to more than 18 hours. Carbohydrate digestion can be performed for more than 1 hour, more than 2 hours, more than 3 hours, more than 4 hours, more than 5 hours, more than 8 hours, more than 10 hours, more than 12 hours, more than 14 hours, more than 16 hours, or more than 18 hours.

Collection matrices can be processed using nucleases. Nucleases can be used to digest genomic DNA or RNA. Non-limiting examples include exonucleases, endonucleases (e.g., restriction enzymes), DNase RNAse, etc. Nuclease digestion can be performed in the presence of one or more buffers. For instance, a buffer can comprise TRIzol® manufactured by Thermofisher®, Buffer RLT manufactured by Qiagen®, Buffer RLN manufactured by Qiagen®, RNA Lysis Buffer (RLA) manufactured by Promega, PureYield™ Cell Lysis Solution (CLA) manufactured by Promega, PureYield™ Endotoxin Removal Wash manufactured by Promega, PureZOL™ RNA isolation reagent (Bio-Rad™), RNA Lysis Buffer or DNA/RNA Binding Buffer manufactured by Zymo Research Corp, or RNA Capture Buffer manufactured by Pierce™, Tris-HCL, MOPS, MES, HEPES, magnesium chloride, calcium chloride, PBS.

Nuclease digestion can be performed for about 10 minutes. Nuclease digestion can be performed for less than 10 minutes, less than 15 minutes, less than 30 minutes, less than 50 minutes, or less than 60 minutes. Nuclease digestion can be performed for less than 1 hour, less than 2 hours, less than 3 hours, less than 4 hours, less than 5 hours, less than 8 hours, less than 10 hours, less than 12 hours, less than 14 hours, less than 16 hours, or less than 18 hours. Nuclease digestion can be performed for more than 10 minutes, more than 15 minutes, more than 30 minutes, more than 50 minutes, or more than 60 minutes. Nuclease digestion can be performed for more than 1 hour to more than 18 hours. Nuclease digestion can be performed for more than 1 hour, more than 2 hours, more than 3 hours, more than 4 hours, more than 5 hours, more than 8 hours, more than 10 hours, more than 12 hours, more than 14 hours, more than 16 hours, or more than 18 hours.

Enzymes can be used for lipid digestion. Enzymatic digestion of lipids can be performed to degrade lipids in a cell. Alternatively, it can be performed to digest a target biomolecule. Examples of such enzymes include lipases, elastase, phospholipases, etc. Lipid digestion can be performed in the presence of buffers. Buffers can be commercially available. Examples of buffers that can be used are presented elsewhere herein.

In some embodiments, more than one enzymatic digestion can be performed on one collection matrix or a combination of collection matrices. The one or more enzymatic digestions can be performed in parallel or one after another. For example, a collection matrix can be processed with a protease and an amylase. In some cases, a carbohydrate digestion processing of a collection matrix can be followed by or performed in parallel a nuclease digestion. In some cases, a nuclease digestion can be performed followed by or in parallel with a lipase digestion. In some cases, more than 2 enzymatic digestions can be performed in parallel. For example, a protease digestion, carbohydrate digestion, nuclease digestion and a lipid digestion can be performed in parallel on a collection matrix.

An enzymatic digestion can be performed in parallel with a mechanical dissociation. Alternatively, an enzymatic digestion can be performed before or after a mechanical dissociation. For example, soaking can be followed by a protease digestion. A nuclease digestion can be performed in parallel with rocking or invert-mixing. Protease and lipid digestions, in some cases, can be followed by sonication. Any other dissociation method presented elsewhere herein can be used in addition to or in parallel to the enzymatic digestion methods.

### 3. Thermal Dissociation

Processing of a collection matrix comprising a biological sample can include a thermally facilitated dissociation. Macromolecules such as proteins, nucleic acids prone to disintegration with temperature can be eluted from the collection matrix with temperature cycling or freeze/thaw cycles or elevated temperatures.

Thermal dissociation processing of a collection matrix can include a low temperature treatment. In some cases, the low temperature treatment includes a freeze/thaw cycle. The low temperature treatment of a collection matrix can include a treatment temperature of about −80° C., about −40° C., about −20° C., about −4° C., about 0° C., or about 4° C. In some cases, the treatment temperature may be less than −80° C., less than −40° C., less than −20° C., less than −4° C., less than 0° C., or less than 4° C. In some cases, the treatment temperature may be more than −80° C., more than −40° C., more than −20° C., more than −4° C., more than 0° C., or more than 4° C.

A thermally facilitated dissociation can include incubating the collection matrix solution at ambient temperatures. Alternatively, the thermally facilitated dissociation can include incubating the collection matrix solution at elevated temperatures. The elevated temperature treatment of a collection matrix can include a treatment temperature of less than 30° C., less than 37° C., less than 45° C., less than 50° C., less than 55° C., less than 60° C., less than 80° C., less than 95° C., less than 97° C., or less than 100° C. In some cases, the elevated temperature treatment of a collection matrix can include a treatment temperature of more than 30° C., more than 37° C., more than 45° C., more than 50° C., more than 55° C., more than 60° C., more than 80° C., more than 95° C., more than 97° C., or more than 100° C.

A thermally facilitated dissociation can include cycling the treatment temperatures. This can include cycling between low temperatures and ambient temperatures. Alternatively, it can include cycling the collection matrix between low temperatures and elevated temperatures or between ambient temperatures and elevated temperatures. A collection matrix can be processed by cycling through a low temperature followed by an elevated temperature followed by ambient temperature incubation or other combinations thereof.

A thermally facilitated dissociation processing procedure can be performed in addition to a mechanical dissociation procedure. The thermal dissociation process can be performed in parallel with mechanical dissociation. For instance, soaking can be performed in parallel with temperature cycling. Also, vortexing can be performed after temperature cycling. Any other mechanical dissociation method presented elsewhere herein can be combined with the thermally facilitated dissociation methods.

A thermally facilitated dissociation processing procedure can be performed in addition to an enzymatic digestion procedure. The thermal dissociation process can be performed in parallel with enzymatic digestion. For instance, nuclease digestion can be performed in parallel with elevated temperature treatment. Also, protease digestion can be performed after temperature cycling. Any other enzymatic digestion method presented elsewhere herein can be combined with the thermally facilitated dissociation methods.

A thermally facilitated dissociation processing procedure can be performed in addition to enzymatic digestion and mechanical dissociation procedures. The thermal dissociation process can be performed in parallel with enzymatic digestion and mechanical dissociation. For instance, nuclease digestion can be performed in parallel with elevated temperature treatment and rocking of the soaked collection matrix.

### 4. Time Dependent Rehydration

Dried collection matrices can be rehydrated. Rehydration of the collection matrices can be performed for different times and temperatures depending on the target biomolecule. For instance, a highly soluble biomolecule can require less rehydration compared to an insoluble biomolecule. In some cases, rehydration can be performed at several different temperatures. Some biomolecules can be soluble at room temperature and others can require higher temperatures. In such cases, rehydration process can include temperature cycling.

Collection matrices can be rehydrated for less than 3 seconds, less than 5 seconds, less than 8 seconds, less than 10 seconds, less than 20 seconds, less than 30 seconds, less than 40 seconds, less than 50 seconds, less than 60 seconds, less than 2 minutes, less than 5 minutes, less than 10 minutes, less than 20 minutes, less than 30 minutes, less than 50 minutes, less than 60 minutes, less than 50 minutes, less than 60 minutes, less than 2 hours, less than 5 hours, less than 8 hours, less than 10 hours, less than 20 hours, less than 30 hours, less than 50 hours, less than 70 hours, less than 80 hours, or less than 100 hours. Collection matrices can be rehydrated for more than 3 seconds, more than 5 seconds, more than 8 seconds, more than 10 seconds, more than 20 seconds, more than 30 seconds, more than 40 seconds, more than 50 seconds, more than 60 seconds, more than 2 minutes, more than 5 minutes, more than 10 minutes, more than 20 minutes, more than 30 minutes, more than 50 minutes, more than 60 minutes, more than 50 minutes, more than 60 minutes, more than 2 hours, more than 5 hours, more than 8 hours, more than 10 hours, more than 20 hours, more than 30 hours, more than 50 hours, more than 70 hours, more than 80 hours, or more than 100 hours.

### 5. Chemical Dissociation and Stabilization

Processing of a collection matrix comprising a biological sample can include a chemically facilitated dissociation and stabilization. Chemical dissociation treatments can comprise introducing a collection matrix to elution buffers. The buffers can comprise various salts, organic solvents, surfactants, protein additives, ion exchange agents, metal chelators, stabilization elements, reducing agents, oxidizing agents or free radical scavengers.

The elution buffers can comprise one or more surfactants. The one or more surfactants can be, e.g., an anionic, cationic, nonionic or amphoteric type. Surfactants used can be able to interact with both hydrophilic and hydrophobic portions of biomolecules and can assist in solubilization and elution of such molecules. The one or more surfactants can be polyethoxylated alcohols; polyoxyethylene sorbitan; octoxynol such as Triton X 100™ (polyethylene glycol p-(1,1,3,3-tetramethylbutyl)-phenyl ether); polysorbates such as Tween™ 20 ((e.g., polysorbate 20) or Tween™ 80 (polysorbate 80); sodium dodecyl sulfate; sodium lauryl sulfate; nonylphenol ethoxylate such as Tergitol™; cyclodextrins; zwitterionic surfactants such as cocamidopropyl betaine. Other betaines include lauramidopropyl betaine, oleamidopropyl betaine, ricinoleamidopropyl betaine, cetyl betaine and dimer dilinoleamidopropyl betaine, sulfobetaines, hydroxysulfobetaines, methylene chloride, and sultaines or any combination thereof. The one or more surfactants can be present at a concentration of less than 0.001%, less than 0.005%, less than 0.01%, less than 0.015%, less than 0.02%, less than 0.025%, less than 0.03%, less than 0.035%, less than 0.04%, less than 0.045%, less than 0.05%, less than 0.055%, less than 0.06%, less than 0.065%, less than 0.07%, less than 0.075%, less than 0.08%, less than 0.085%, less than 0.09%, less than 0.095%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, or less than 0.1% by volume relative to the total volume of the elution buffer. The one or more surfactants can be at a concentration of about 0.01%, 0.05%, 0.1%, 0.5%, 1%, 5%, or 10%. The one or more surfactants can be at a concentration of less than 0.01%, 0.05%, 0.1%, 0.5%, 1%, 5%, or 10%. The one or more surfactants can be at a concentration of more than 0.01%, 0.05%, 0.1%, 0.5%, 1%, 5%, or 10%.

The elution buffer can comprise one or more organic solvent mixtures. Organic extraction with aqueous and organic solvent mixtures can function to solubilize and elute biomolecules. An organic solvent can comprise butanol, ethanol, methanol, isopropanol, phenol, propanol, DMSO, DMF, dioxane, tetrahydrofuran, butanol, t-butanol, pentanol, acetone, chloroform or a combination thereof. The elution buffer can comprise less than 0.01%, less than 0.05%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, less than 1%, less than 1.5%, less than 2%, less than 2.5%, less than 3%, less than 3.5%, less than 4%, less than 4.5%, less than 5%, less than 5.5%, less than 6%, less than 6.5%, less than 7%, less than 7.5%, less than 8%, less than 8.5%, less than 9%, less than 9.5%, less than 10%, less than 11%, less than 12%, less than 13%, less than 14%, less than 15%, less than 16%, less than 17%, less than 18%, less than 19%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 99%, or 100% of an organic solvent by volume relative to the total volume of the solution. The concentration of the one or more organic solvents in the elution buffer can be at least 1%, 5%, 10%, 50%, 75%, or 100%. The concentration of the one or more organic solvents in the elution buffer can be about 1%, 5%, 10%, 50%, 75%, or 100%.

Buffers can include chaotropic agents such as guanidine chloride, guanidine hydrochloride, guanidine isothiocyanate, lithium perchlorate, lithium acetate, magnesium chloride, sodium iodide, sodium thiocyanate, thiourea, urea, or any combination thereof. The concentration of the chaotropic agent in a buffer can be about 0.1 mM, 1 mM, 10 mM, 100 mM, 1 M, 6 M, or 8 M. The concentration of the chaotropic agent in a buffer can be at least 0.1 mM, 1 mM, 10 mM, 100 mM, 1 M, 6 M, or 8 M. The concentration of the chaotropic agent in a buffer can be less than 0.1 mM, 1 mM, 10 mM, 100 mM, 1 M, 6 M, or 8 M.

Chemically facilitated dissociation and stabilization can comprise addition of protein and/or nucleic acid additives to the elution buffer. Adding proteins and/or nucleic acids to the elution-solution can serve to drive the biomolecules of interest off a polysaccharide coated collection matrix with competitive binding of the collection matrix. Additionally, additives can stabilize biomolecules and block non-specific binding of biomolecules to labware. Examples of additives include, but are not limited to: BSA, albumin, casein, dry milk, non-fat milk, egg-white, non-human serum, blood substitutes, nucleic acids, yeast RNA, herring sperm DNA, salmon sperm DNA, calf thymus DNA, COT-1 DNA, synthetic oligonucleotides. The elution buffer can comprise less than 0.0001%, less than 0.005%, less than 0.01%, less than 0.05%, less than 0.1%, less than 0.15%, less than 0.2%, less than 0.25%, less than 0.3%, less than 0.35%, less than 0.4%, less than 0.45%, less than 0.5%, less than 0.55%, less than 0.6%, less than 0.65%, less than 0.7%, less than 0.75%, less than 0.8%, less than 0.85%, less than 0.9%, less than 0.95%, less than 1%, less than 1.5%, less than 2%, less than 2.5%, less than 3%, less than 3.5%, less than 4%, less than 4.5%, less than 5%, less than 5.5%, less than 6%, less than 6.5%, less than 7%, less than 7.5%, less than 8%, less than 8.5%, less than 9%, less than 9.5%, less than 10%, less than 11%, less than 12%, less than 13%, less than 14%, less than 15%, less than 16%, less than 17%, less than 18%, less than 19%, less than 20%, less than 25%, less than 30%, less than 35%, less than 40%, less than 45%, less than 50%, less than 55%, less than 60%, less than 65%, less than 70% of a protein, nucleic acid, or protein/nucleic acid mixture by volume relative to the total volume of the solution. The concentration of the one or more proteins, nucleic acid, or protein/nucleic acid mixtures in the elution buffer can be at least 0.0001%, 0.005%, 0.001%, 0.05%, 1%, 5%, 10% or 50%. The concentration of the one or more proteins, nucleic acid, or protein/nucleic acid mixtures in the elution buffer can be about 0.0001%, 0.005%, 0.001%, 0.05%, 1%, 5%, 10% or 50%.

Chemically facilitated dissociation and stabilization can comprise elution buffers comprising ion exchange agents. Ion exchange agents can comprise any agents that can affect the ionic strength of proteins. The ionic strength can be affected due to a change in the solubility, activity, binding or stabilization properties of biomolecules. The one or more salts can be sodium chloride, sodium acetate, sodium bicarbonate, sodium bisulfate, sodium bromide, magnesium chloride, magnesium acetate, calcium chloride, potassium chloride, potassium acetate, potassium bicarbonate, potassium bisulfate, potassium bromate, potassium bromide, or potassium carbonate. The one or more salts can be at a concentration of about 0.1 mM, 5 mM, 10 mM, 25 mM, 50 mM, 100 mM, 250 mM, 500 mM, or 750 mM. The one or more salts can be at a concentration of less than 0.1 mM, 5 mM, 10 mM, 25 mM, 50 mM, 100 mM, 250 mM, 500 mM, or 750 mM. The one or more salts can be at a concentration of at least 0.1 mM, 5 mM, 10 mM, 25 mM, 50 mM, 100 mM, 250 mM, 500 mM, 750 mM, or 1000 mM.

Ion exchange reagents can comprise one or more buffering agents. The one or more buffering agents can be, e.g., saline, citrate, phosphate, phosphate buffered saline, acetate, glycine, tris(hydroxymethyl)aminomethane (tris) hydrochloride, tris buffered saline (TBS), 3-[[1,3-dihydroxy-2-(hydroxymethyl)propan-2-yl]amino]propane-1-sulfonic acid (TAPS), bicine, tricine, 3-[[1,3-dihydroxy-2-(hydroxymethyl)propan-2-yl]amino]-2-hydroxypropane-1-sulfonic acid (TAPSO), 4-(2-hydroxyethyl)-1-piperazineethanesulfonic acid (HEPES), piperazine-N,N′-bis(2-ethanesulfonic acid) (PIPES), 3-(N-morpholino)propanesulfonic acid (MOPS), 2-(N-morpholino)ethanesulfonic acid (MES), 2-[[1,3-dihydroxy-2-(hydroxymethyl)propan-2-yl]amino]ethanesulfonic acid (TES), cacodylate, glycine, carbonate, or any combination thereof. The buffering agent can be present at a concentration of less than 500 mM, less than 400 mM, less than 300 mM, less than 200 mM, less than 100 mM, less than 50 mM, less than 25 mM, less than 20 mM, less than 15 mM, less than 10 mM, less than 5 mM, less than 4 mM, less than 3 mM, less than 2 mM, less than 1 mM, less than 0.9 mM, less than 0.8 mM, less than 0.7 mM, less than 0.6 mM, less than 0.5 mM, less than 0.4 mM, less than 0.3 mM, less than 0.2 mM, or less than 0.1 mM. The buffering agent can be present at a concentration of more than 500 mM, more than 400 mM, more than 300 mM, more than 200 mM, more than 100 mM, more than 50 mM, more than 25 mM, more than 20 mM, more than 15 mM, more than 10 mM, more than 5 mM, more than 4 mM, more than 3 mM, more than 2 mM, more than 1 mM, more than 0.9 mM, more than 0.8 mM, more than 0.7 mM, more than 0.6 mM, more than 0.5 mM, more than 0.4 mM, more than 0.3 mM, more than 0.2 mM, or more than 0.1 mM.

Chemically facilitated dissociation and stabilization can comprise pH facilitated treatment. pH facilitated chemical dissociation can include pH cycling. For example, an elution buffer can initially be a more basic solution with pH ranging between 9 and 12. Salts or acids can be added to the elution buffer cycling the buffer from basic to acidic.

The pH of the elution buffer can be about 1 to about 14. The pH of the elution buffer can be at least about 1. The pH of the elution buffer can be at most about 14. The pH of the elution buffer can be less than 2, less than 3, less than 4, less than 5, less than 6, less than 7, less than 8, less than 9, less than 10, less than 11, less than 12, or less than 14. The pH of the elution buffer can be more than 1, more than 3, more than 4, more than 5, more than 6, more than 7, more than 8, more than 9, more than 10, more than 11, more than 12, or more than 13.

Chemically facilitated dissociation and stabilization of a collection matrix can comprise treatment with chelating agents. The one or more chelators can be, e.g., a carbohydrate; a lipid; a steroid; an amino acid or related compound; a phosphate; a nucleotide; a tetrapyrrol; a ferrioxamines; an ionophor; a phenolic; or a synthetic chelator such as 2,2′-bipyridyl, dimercaptopropanol, ethylenediaminotetraacetic acid (EDTA), ethylenedioxy-diethylene-dinitrilo-tetraacetic acid, ethylene glycol-bis-(2-aminoethyl)-N,N,N′, N′-tetraacetic acid (EGTA), a metal nitrilotriacetic acid (NTA), salicylic acid, citrate or triethanolamine (TEA). The concentration of the one or more chelating agents in a buffer can be about 0.01 mM, 0.1 mM, 1 mM, 5 mM, 10 mM, 20 mM, or 25 mM. The concentration of the one or more chelating agents in a buffer can be less than 0.1 mM, 1 mM, 5 mM, 10 mM, 20 mM, or 25 mM. The concentration of the one or more chelating agents in a buffer can be more than 0.1 mM, 1 mM, 5 mM, 10 mM, 20 mM, or 25 mM.

Chemically facilitated dissociation and stabilization of a collection matrix can comprise treatment with agents that can prevent aggregation. Aggregation preventing agents can comprise polyols. The one or more polyols can be a glycol such as ethylene glycol or propylene glycol, or a glycol polymer such as polyethylene glycol (PEG) of various weights such as PEG300, PEG400, PEG600, PEG1000, PEG3000, PEG6000, PEG8000, or PEG10000. In some instances, the one or more polyols can be a sugar. In some cases, the sugar can be sucrose, glucose, fructose, trehalose, maltose, melezitose, galactose, lactose or any combination thereof. In some instances, the one or more polyols can be a sugar alcohol. In some cases, the sugar alcohol can be glycerol, erythritol, threitol, xylitol, sorbitol, etc. The concentration of aggregation preventing agents in an elution buffer can be about 0.5%, about 1%, about 2%, about 5%, about 10%, about 15%, about 20%, about 25%, about 30%, about 40%, about 50% or about 60%. The concentration of aggregation preventing agents in an elution buffer can be less than 0.5%, less than 1%, less than 2%, less than 5%, less than 10%, less than 15%, less than 20%, less than 25%, less than 30%, less than 40%, less than 50% or less than 60%. The concentration of aggregation preventing agents in an elution buffer can be more than 0.5%, more than 1%, more than 2%, more than 5%, more than 10%, more than 15% more than 20%, more than 25%, more than 30%, more than 40%, more than 50% or more than 60%.

Elution buffers can comprise one or more reducing or oxidizing agents. Reducing agents can reduce or oxidize biomolecules by altering their solubility, activity, binding and stabilization properties of biomolecules. The one or more reducing or oxidizing agents can be, e.g., beta-mercaptoethanol (BME), 2-aminoethanethiol (2MEA-HCl (cysteamine-HCl)), dithiothreitol (DTT), glutathione (GSH), glutathione disulfide (GSSG), tris(2-carboxyethyl)phosphine (TCEP), NADPH, ascorbic acid, retinoic acid, and tocopherols or any combination thereof. The concentration of the one or more reducing agents can be about 0.1 mM, 0.5 mM, 1 mM, 10 mM, 50 mM, 100 mM, 250 mM, or 500 mM. The concentration of the one or more reducing or oxidizing agents can be less than 0.1 mM, 0.5 mM, 1 mM, 10 mM, 50 mM, 100 mM, 250 mM, or 500 mM. For example, the concentration of DTT can be from less than 0.05 mM to less than 100 mM, from less than 0.5 mM to less than 50 mM, or from less than 5 mM or less than 10 mM. The concentration of TCEP can be less than 0.05 mM, less than 5 mM, less than 10 mM or less than 50 mM. The concentration of BME can be from less than 0.05%, less than 5%, or less than 10%. The concentration of GSH can be from less than 0.05 mM, less than 5 mM or less than 10 mM. The concentration of the one or more reducing or oxidizing agents can be about 1 mM, 10 mM, 50 mM, 100 mM, 250 mM, or 500 mM.

Elution buffers can comprise one or more free radical scavengers. Radical scavengers can include hydoquinone derivatives including tetrahydroxy-1,4-benzoquinone (THQ) or Mono Methyl Ether of Hydroquinone; (MEHQ), glutathione (GSH), ascorbic acid, retinoic acid, and tocopherols. The concentration of the one or more free radical scavengers in a buffer can be about 0.1 mM, 1 mM, 5 mM, 10 mM, 20 mM, 25 mM, 27 mM, 28 mM, 29 mM, 30 mM, 32 mM, 35 mM, 38 mM, 40 mM, 45 mM, 50 mM, or 100 mM. The concentration of the one or more free radical scavengers in a buffer can be less than 0.1 mM, 1 mM, 5 mM, 10 mM, 20 mM, 25 mM, 27 mM, 28 mM, 29 mM, 30 mM, 32 mM, 35 mM, 38 mM, 40 mM, 45 mM, 50 mM, or 100 mM. The concentration of the one or more free radical scavenger agents in a buffer can be more than 0.1 mM, 1 mM, 5 mM, 10 mM, 20 mM, 25 mM, 27 mM, 28 mM, 29 mM, 30 mM, 32 mM, 35 mM, 38 mM, 40 mM, 45 mM, 50 mM, or 100 mM.

A chemically facilitated dissociation and stabilization procedure can be performed in addition to or in parallel other dissociation methods. A chemical dissociation can be performed in parallel with rocking the collection matrix or vortexing the collection matrix solution. A chemical dissociation method can be performed before or after an enzymatic dissociation. For example, a carbohydrate digesting enzyme can first degrade the polysaccharide coating on the collection matrix followed by treatment with an elution buffer to elute nucleic acids. A chemical dissociation method can be performed at different temperatures or in addition to cycling different temperatures to facilitate thermal dissociation. A chemical dissociation method can be performed in addition to or in parallel with any of the mechanical dissociation, enzymatic dissociation, thermally facilitated dissociation or time dependent rehydration and dissociation methods presented elsewhere herein.

The collection matrix, or the portion of the collection matrix, can be contacted with a volume of the elution buffer of less than 5 μL, less than 10 μL, less than 15 μL, less than 20 μL, less than 25 μL, less than 30 μL, less than 35 IL, less than 40 μL, less than 45 μL, less than 50 μL, less than 55 μL, less than 60 μL, less than 65 μL, less than 70 μL, less than 75 μL, less than 80 μL, less than 85 μL, less than 90 μL, less than 95 μL, less than 100 μL, less than 110 μL, less than 120 μL, less than 130 μL, less than 140 μL, less than 150 μL, less than 160 μL, less than 170 μL, less than 180 μL, less than 190 μL, less than 200 μL, less than 250 μL, less than 300 μL, less than 350 μL, less than 400 μL, less than 450 μL, less than 500 μL, less than 550 μL, less than 600 μL, less than 650 μL, less than 700 μL, less than 750 μL, less than 800 μL, less than 850 μL, less than 900 μL, less than 950 μL, less than 1,000 μL, less than 1.5 mL, less than 2 mL, less than 2.5 mL, less than 3 mL, less than 3.5 mL, less than 4 mL, less than 4.5 mL, less than 5 mL, less than 5.5 mL, less than 6 mL, less than 6.5 mL, less than 7 mL, less than 7.5 mL, less than 8 mL, less than 8.5 mL, less than 9 mL, less than 9.5 mL, or less than 10 mL. The stabilization collection matrix, or portion of the stabilization collection matrix, can be contacted with about 0.1 mL, 0.2 mL, 0.5 mL, 0.7 mL, 1 mL, 2 mL, 5 mL, 7 mL, or 10 mL of buffer.

The volume of elution buffer contacted with the collection matrix can be dependent on the surface area of the collection matrix. The amount of elution buffer can be less than 1 μL/mm2, less than 2 μL/mm2, less than 3 μL/mm2, less than 4 μL/mm2, less than 5 μL/mm2, less than 6 μL/mm2, less than 7 μL/mm2, less than 8 μL/mm2, less than 9 μL/mm2, less than 10 μL/mm2, less than 12 μL/mm2, less than 14 μL/mm2, less than 16 μL/mm2, less than 18 μL/mm2, less than 20 μL/mm2, less than 25 μL/mm2, less than 30 μL/mm2, less than 35 μL/mm2, less than 40 μL/mm2, less than 45 μL/mm2, less than 50 μL/mm2, less than 55 μL/mm2, less than 60 μL/mm2, less than 65 μL/mm2, less than 70 μL/mm2, less than 75 μL/mm2, less than 80 μL/mm2, less than 85 μL/mm2, less than 90 μL/mm2, less than 95 μL/mm2, less than 100 μL/mm2, less than 150 μL/mm2, less than 200 μL/mm2, less than 250 μL/mm2, less than 300 μL/mm2, less than 350 μL/mm2, less than 400 μL/mm2, less than 450 μL/mm2, less than 500 μL/mm2, less than 550 μL/mm2, less than 600 μL/mm2, less than 650 μL/mm2, less than 700 μL/mm2, less than 750 μL/mm2, less than 800 μL/mm2, less than 850 μL/mm2, less than 900 μL/mm2, less than 950 μL/mm2, or less than 1,000 μL/mm2.

Non-limiting embodiments of the sample stabilization unit can employ sample separation components to separate other non-plasma or non-serum components as well. Sample separation components can be connected to the sample acquisition component e.g., through channels, including microchannels, wicking of absorbent materials or other means that allow sample to flow through the device. The systems and methods for separating the sample are exemplary and non-limiting.

Generally, a sample can contain or is suspected of containing one or more analytes. The term “analyte” as used herein can refer to any substance that can be analyzed using the assays or immunoassay devices. As an example, an immunoassay device can be configured to detect the presence of 1, 2, 3, 4, 5, 6, 7, 8, 9, 10 or more analytes in a sample. Non-limiting examples of analytes can include proteins, haptens, immunoglobulins, hormones, polynucleotides, steroids, drugs, infectious disease agents (e.g., of bacterial or viral origin), drugs of abuse, environmental agents, biological markers, and the like.

### F. Example of a Cartridge Assembly

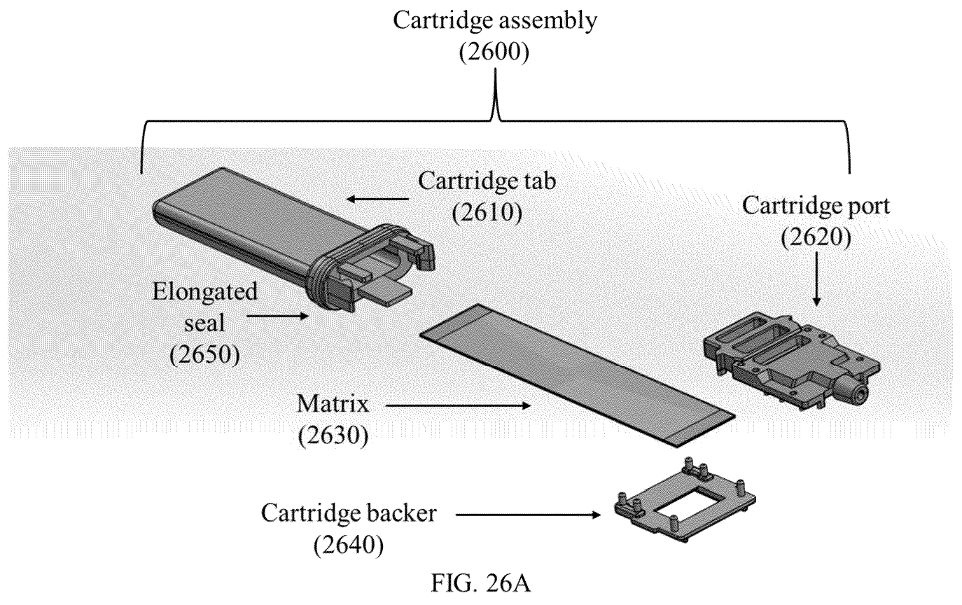

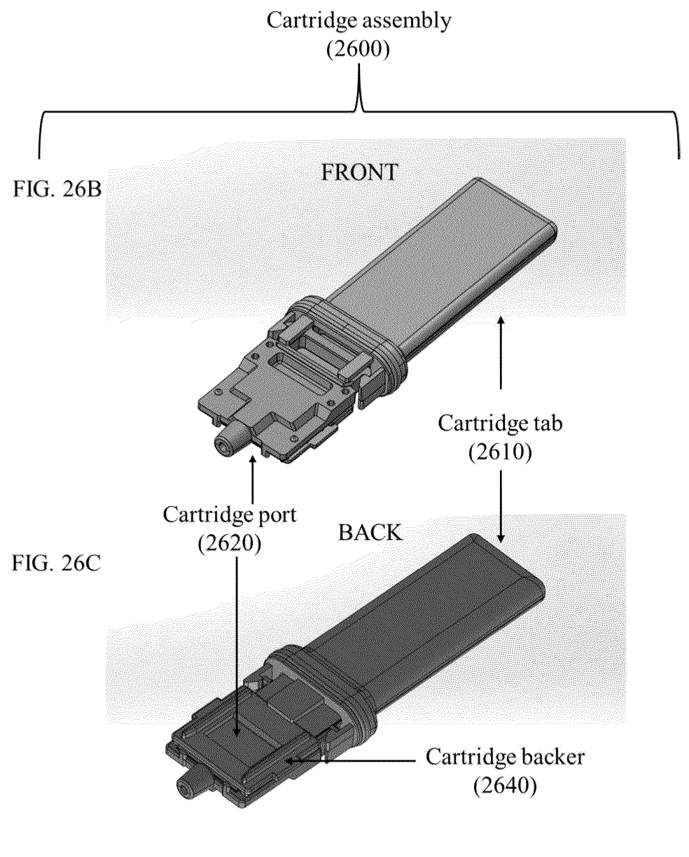

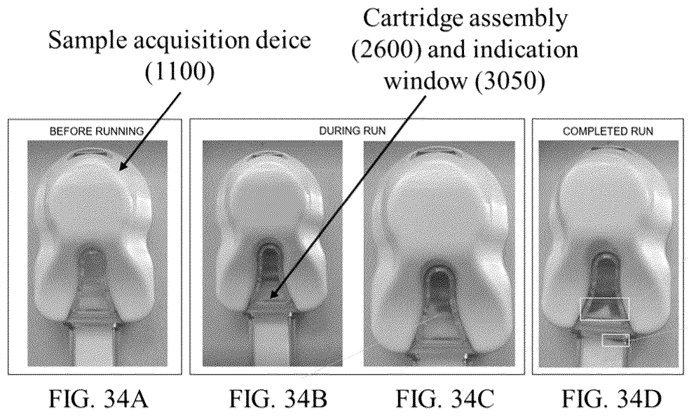

FIGS. 26A-26C illustrate a nonlimiting embodiment of a cartridge assembly 2600 which can be used with, for example, a sample acquisition device 1100 as shown in FIGS. 34A-34D. The cartridge assembly 2600 can comprise one or more features. For example, the cartridge assembly can comprise an elongated housing (e.g., cartridge tab) 2610; an inlet component (e.g., a cartridge port) 2620; a treatment/stabilization unit (e.g., an elongated strip or a matrix described elsewhere herein) 2630; or a cartridge backer (e.g., a backing plate) 2640. The cartridge tab 2610 can include an elongated seal 2650. In some embodiments, the matrix 2630 is supported (e.g., sandwiched) between the cartridge tab 2610, the cartridge port 2620, and the cartridge backer 2640.

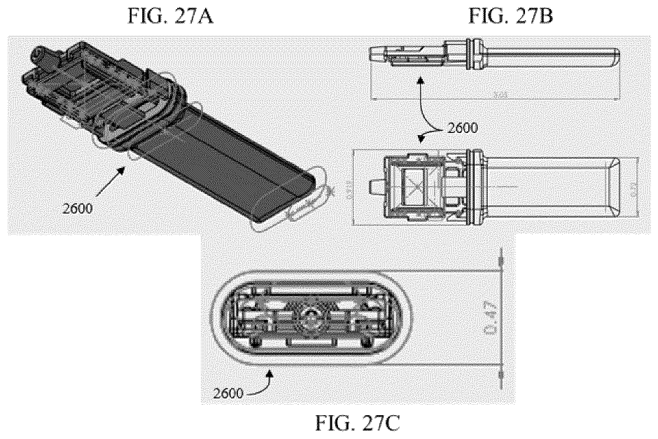

FIGS. 27A-27C illustrate nonlimiting dimensions of the cartridge assembly 2600. For example, as shown in FIG. 27B, the cartridge assembly 2600 may have a length ranging from about 1.5 inches to about 4.5 inches. In some preferred embodiments, the length may be about 2.5 inches to about 3.6 inches. The length may be measured from a distal end of the cartridge tab 2610 to a distal end of the cartridge port 2620. The cartridge assembly 2600 may have a first width ranging from about 0.5 inches to about 1.4 inches. The first width may be at an end of the cartridge assembly 2600 having the cartridge port 2620. The first width may be measured from a side of the cartridge port 2620 to an opposite side of the cartridge port 2620. In some preferred embodiments, the first width may be about 0.75 inches to about 1.1 inches. The cartridge assembly may have a second width ranging from about 0.40 inches to about 1.1 inches. The second width may be at an end of the cartridge assembly 2600 having the cartridge tab 2610. The second width may be measured from a side of the cartridge tab 2610 to an opposite side of the cartridge tab 2610. In some preferred embodiments, the second width may be about 0.60 inches to about 0.90 inches.

The cartridge assembly 2600 may have an area ranging from about 1.4 in² to about 4.2 in². In some preferred embodiments, the area may be about 2.2 in² to about 3.4 in². The cartridge assembly 2600 may have a ratio of length to width ranging from about 1.7 to about 6.4. In some preferred embodiments, the ratio of length to width may be about 3.4 to 5.0. The length to width ratio of the cartridge assembly 2600 may be configured to and compatible with a matrix 2630 having a length to width ratio to yield quality plasma. The cartridge assembly 2600 may have a thickness ranging from about 0.25 inches to about 0.7 inches. The thickness may be measured from a side of the elongated seal 2650 to an opposite side of the elongated seal 2650. In some preferred embodiments, the thickness may be about 0.40 inches to about 0.60 inches.

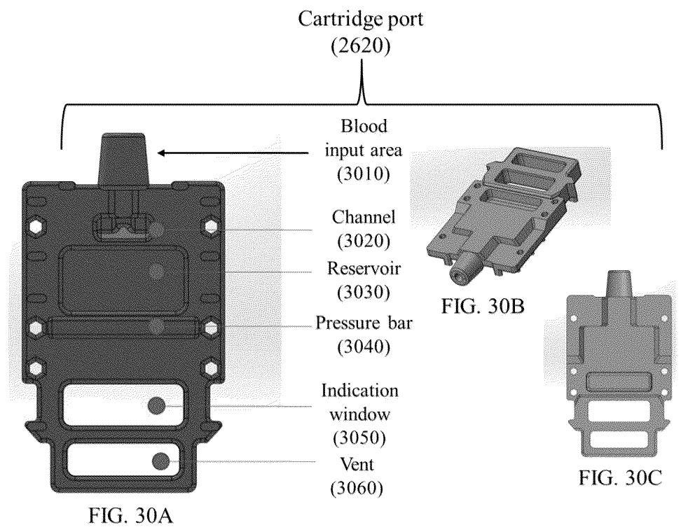

FIGS. 30A-30C illustrate a nonlimiting embodiment of the inlet component (e.g., cartridge port) 2620. The cartridge port 2620 can comprise one or more features. For example, the cartridge port 2620 can comprise features of a blood input area (e.g., a port to introduce a sample e.g., blood into other features of the cartridge assembly) 3010 described elsewhere herein; turn features (e.g., channels) 3020 described elsewhere herein; a reservoir 3030 described elsewhere herein; a pressure bar 3040 described elsewhere herein, an indication window 3050 described elsewhere herein; or a vent 3060 described elsewhere herein.

The blood input area 3010 can comprise a port configured to receive a sample e.g., blood via the sample acquisition device 1100. The port may have, for example, a tapered profile ranging from about 0 degrees to about 45 degrees. In some preferred embodiments, the tapered profile may be about 15 degrees to about 30 degrees. The port may have a diameter that varies along a length of the port. The diameter can range from about 0.10 inches to about 0.3 inches at a surface proximal to an edge of the cartridge port 2620. In some preferred embodiments, the diameter may be about 0.15 inches to about 0.25 inches. The diameter can range from about 0.05 inches to about 0.20 inches at a surface opposite to an edge of the cartridge port 2620. In some preferred embodiments, the diameter may be about 0.1 inches to about 0.15 inches. The tapered profile of the port may be configured to and compatible with a matrix 2630 having a length to width ratio to yield quality plasma.

The blood input area 3010 can comprise a port having a surface area in contact with a flow of a sample (e.g., flow of blood) configured to, for example, prevent clogging of the matrix 2630, generate minimal plasma loss, or generate maximum plasma yield and quality. The surface area may be optimized to a matrix having a determined length to width ratio described elsewhere herein. The channel 3020 may comprise one or more channels configured to have one or more turns, described elsewhere herein, that introduce a sample (e.g., blood) via the port onto the matrix 2630. The one or more channels may be configured to introduce blood onto the matrix in an orthogonal manner to, for example, prevent blood from running down a surface of the matrix when vertically oriented on a skin of a subject. In some cases, the one or more channels may be configured to induce a change in a direction of the flow of blood to counteract gravitational force on the flow of blood. The reservoir 3030 may be configured to, for example, collect, aggregate, or pool blood while wicking occurs on the matrix 2630. The reservoir 3030 may be configured to be located adjacent to one or more turn features of the channel 3020. In some cases, the one or more turn features are located between the port (e.g., the blood input area 3010) and the reservoir 3030. The reservoir 3030 may be located between the pressure bar 3040 and the one or more turn features of the channel 3020. The pressure bar 3040 may be configured to, for example, enable a vertical orientation of the cartridge assembly 2600 by slowing blood to ensure wicking on the matrix 2630 and optimization of plasma separation and yield. The pressure bar 3040 may be configured to regulate a flow speed of the blood sample and ensure proper wicking of the blood sample along the matrix 2630 for optimal separation of the plasma from the blood sample. The pressure bar 3040 may be located adjacent to the reservoir 3030. The indication window 3050 may be configured to provide an indication for a user to, for example, remove the sample acquisition device 1100 from a subject's body when sample acquisition is complete. The indication window 3050 may be configured to view progress of the blood plasma separation on the matrix 2630. The vent 3060 may be configured to, for example, ensure a vacuum flows freely through the matrix 2630, into the reservoir 3030, and throughout the elongated seal 2650 to maintain system continuity (e.g., flow of blood through the cartridge assembly 2600).

The pressure bar 3040 may be configured to reduce pressure or increase pressure at or near one or more regions of a flow path of a sample (e.g., blood) inside the cartridge assembly 2600. The pressure bar 3040 may be configured with different dimensions, shapes, features, materials, or any combination thereof to be compatible with samples, treatments, and/or chemical agents. The pressure bar 3040 may be configured to increase pressure (e.g., a squeezing feature) at or near one or more regions of a flow path of a sample. The squeezing feature may be configured to squeeze plasma from a sample (e.g., blood) to optimize plasma yield for an otherwise smaller surface area of the matrix 2630. In some cases, the squeezing feature may be configured as a stop that can be lowered or raised to restrict, stop, or substantially stop blood flow and to intentionally isolate plasma across regions of flow paths of samples (e.g., blood). The pressure bar 3040 may configured to reduce pressure (e.g., a relief feature) at or near one or more regions of a flow path of a sample. The relief feature may be configured as a notch to increase flow of blood. The relief feature may be configured as a notch to take over a flow when enough sample (e.g., blood) has been collected from a user using the sample acquisition device 1100. In some cases, the pressure bar may be configured to lower, raise, or otherwise affect the position of the notch or the stop. The pressure bar 3040 may be configured with perforated areas to provide for easier end use processing. The perforated areas may be etched, lasered, mechanically punched, or any combination thereof.

The pressure bar 3040 may be configured in conjunction with the stand offs 3130 to form a gap between the cartridge port 2620 and cartridge backer 2640. Alternatively, the pressure bar 3040 may be configured to provide the gap. The size of the gap may range from about 0 mm to about 4 mm. The gap may be configured to be adjustable or fixed. The size of the gap may be substantially constant across a width or length of the gap. The size of the gap may be variable across a width or length of the gap. The pressure bar 3040 may be configured in combination with the tapered port of the cartridge port 2620 to yield quality plasma.

The pressure bar 3040 may be configured to provide automatic operation during sample acquisition (e.g., blood). For example, the pressure bar 3040 may be configured to automatically increase pressure or decrease pressure to stop, decrease, or increase flow of a sample (e.g., blood) during sample acquisition. For example, the pressure bar 3040 may be configured to automatically stop flow during overflow scenarios when a user leaves the sample acquisition device 1100 on for more than a predetermined time (e.g., more than about 20 mins). For example, the pressure bar 3040 may be configured to automatically increase flow during sample acquisition underflow scenarios when not enough sample is being collected by the sample acquisition device 1100. For example, the pressure bar 3040 may be configured to automatically decrease flow during sample acquisition sample acquisition overflow scenarios when too much sample is being collected by the sample acquisition device. For example, the pressure bar 3040 may be configured to provide automatic multiplexing and processing of different pieces of sample collection materials in various sample collection tubes without needing to configure new materials. For example, the pressure bar 3040 may be configured to automatically collect plasma in as small of a surface area and/or volume of material as possible. The pressure bar 3040 may be agnostic to chemical treatments, surface treatments, or overall device dimensions. The pressure bar 3040 may be configured for ease of manufacturing.

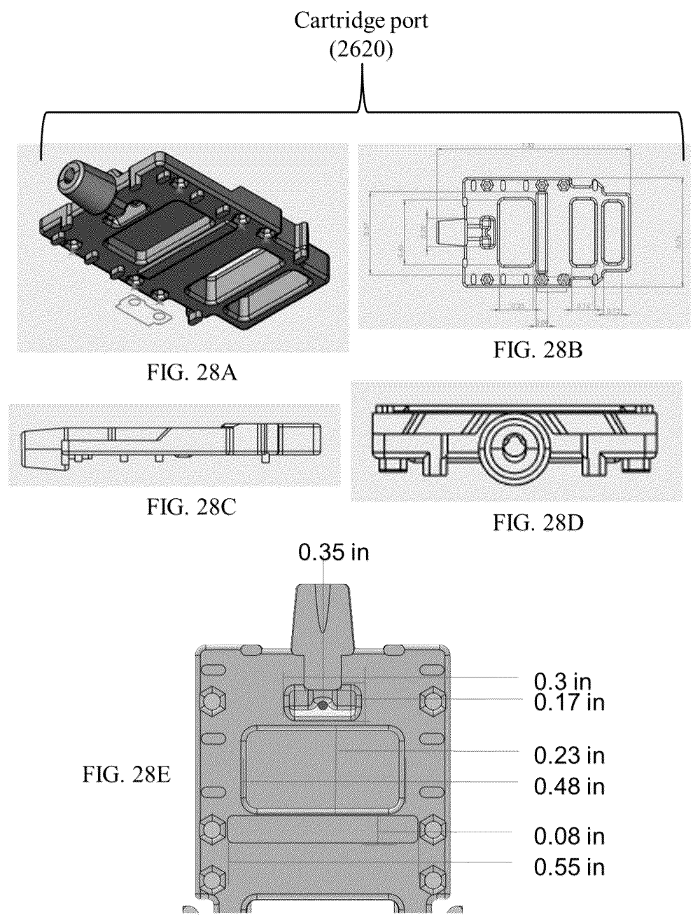

FIGS. 28A-28E illustrate nonlimiting dimensions of the cartridge port 2610. For example, as shown in FIG. 28B, the cartridge port 2610 may have a length ranging from about 0.7 inches to about 2.0 inches. The length may be measured from an end of the cartridge port 2610 to an opposite end of the cartridge port 2610. In some preferred embodiments, the length may be about 1.1 inches to about 1.6 inches. The cartridge port 2610 may have a width ranging from about 0.4 inches to about 1.1 inches. The width may be measured from a side of the cartridge port 2610 to an opposite side of the cartridge port 2610. In some preferred embodiments, the width may be about 0.6 inches to about 0.9 inches. The cartridge port 2610 may have a thickness ranging from about 0.1 inches to about 0.3 inches. The thickness may be measured from a side of the cartridge port 2610 to an opposite side of the cartridge port. In some preferred embodiments, the thickness is about 0.16 inches to about 0.24 inches. The cartridge port 2610 may have a surface area ranging from about 0.5 in² to about 1.5 in². In some preferred embodiments, the surface area may be about 0.8 in² to about 1.2 in². The cartridge port 2610 may have a length to width ratio ranging from about 0.9 to about 2.7. In some preferred embodiments, the length to width ratio may be about 1.4 to about 2.1. The length to width ratio of the cartridge port 2610 may be configured to and compatible with a matrix 2630 having a length to width ratio to yield quality plasma.

The reservoir 3030 of the cartridge port 2620 may have a width ranging from about 0.2 inches to about 0.7 inches. The width may be measured from a side of the revoir 3030 to an opposite side of the reservoir 3030. In some preferred embodiments, the width may be about 0.4 inches to about 0.5 inches. The reservoir may have a length ranging from about 0.1 inches to about 0.3 inches. The length may be measured from an end of the reservoir 3030 to an opposite end of the reservoir 3030. In some preferred embodiments, the length may be about 0.2 inches to about 0.3 inches. The reservoir 3030 may have a surface area ranging from about 0.05 in² to about 0.16 in². In some preferred embodiments, the surface area may be about 0.08 in² to about 0.12 in². The reservoir 3030 may have a length to width ratio ranging from about 0.25 to about 0.75. In some preferred embodiments, the length to width ratio may be about 0.4 to about 0.6. The length to width ratio of the reservoir 3030 may be configured to and compatible with a matrix 2630 having a length to width ratio to yield quality plasma.

The reservoir 3030 may be located a distance from an edge of the reservoir 3030 to the pressure bar 3040 having a range of about 0 mm to about 5 mm. In some preferred embodiments, the distance may be about 0 mm. The reservoir 3030 may have a volume ranging from about 30 mm² to about 300 mm². In some preferred embodiments, the volume may be about 175 mm². An edge of the matrix 2630 may be configured to extend into the reservoir 3030. An edge of the matrix 2630 may be configured to extend to and substantially align with an edge of the reservoir 3030.

The indication window 3050 of the cartridge port 2620 may have a width ranging from about 0.25 inches to about 0.7 inches. The width may be measured from a side of the indication window 3050 to an opposite side of the indication window 3050. In some preferred embodiments, the width may be about 0.35 inches to about 0.55 inches. The indication window 3050 may have a length ranging from about 0.08 inches to about 0.25 inches. The length may be measured from an end of the indication window 3050 to an opposite end of the indication window 3050. In some preferred embodiments, the length may be about 0.13 inches to about 0.19 inches. The indication window 3050 may have a surface area ranging from about 0.04 in² to about 0.11 in². In some preferred embodiments, the surface area may be about 0.06 in² to about 0.09 in². The indication window 3050 may have a length to width ratio ranging from about 0.18 to about 0.53. In some preferred embodiments, the length to width ratio may be about 0.28 to about 0.43. The length to width ratio of the indication window 3050 may be configured to and compatible with a matrix 2630 having a length to width ratio to yield quality plasma.

The vent 3060 of the cartridge port 2620 may have a width ranging from about 0.25 inches to about 0.7 inches. The width may be measured from a side of the vent 3060 to an opposite side of the vent 3060. In some preferred embodiments, the width may be about 0.35 inches to about 0.55 inches. The vent 3060 may have a length ranging from about 0.06 inches to about 0.18 inches. The length may be measured from an end of the vent 3060 to an opposite end of the vent 3060. In some preferred embodiment, the length may be about 0.10 inches to about 0.14 inches. The vent 3060 may have a surface area ranging from about 0.03 in² to about 0.08 in². In some preferred embodiments, the surface area may be about 0.04 in² to about 0.06 in². The vent 3060 may have a length to width ratio ranging from about 0.13 to 0.40. In some preferred embodiments, the length to width ratio may be about 0.21 to about 0.32. The length to width ratio of the vent 3060 may be configured to and compatible with a matrix 2630 having a length to width ratio to yield quality plasma.

The pressure bar 3040 of the cartridge port 2620 may have a width ranging from about 0.3 inches to about 0.9 inches. The width may be measured from a side of the pressure bar 3040 to an opposite side of the pressure bar 3040. In some preferred embodiments, the width may be about 0.45 inches to about 0.7 inches. The pressure bar 3040 may have a length ranging from about 0.04 inches to about 0.12 inches. The length may be from an end of the pressure bar 3040 to an opposite end of the pressure bar 3040. In some preferred embodiments, the length may be about 0.06 inches to about 0.10 inches. The pressure bar 3040 may have a surface area ranging from about 0.02 in² to about 0.07 in². In some preferred embodiments, the surface area may be about 0.04 in² to about 0.05 in². The pressure bar 3040 may have a length to width ratio ranging from about 0.07 to about 0.21. In some preferred embodiments, the length to width ratio may be about 0.11 to about 0.17. The length to width ratio of the pressure bar 3040 may be configured to and compatible with a matrix 2630 having a length to width ratio to yield quality plasma. The pressure bar 3040 may be located at a distance ranging from about 30 mm to about 90 mm from a distal end of the matrix 2630 such that the pressure bar 3040 is located along the matrix 2630.

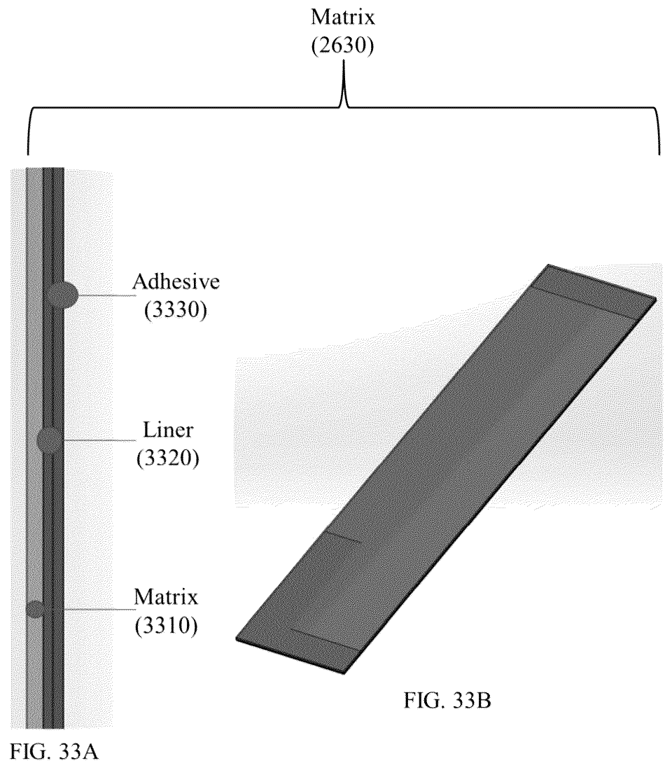

FIGS. 33A-33B illustrate a nonlimiting embodiment of the treatment/stabilization unit 2630 (e.g., a matrix described elsewhere herein). The matrix 2630 can comprise one or more features. For example, the matrix 2630 can comprise a substrate, one or more matrices 3310, one or more liners 3320, or one or more adhesives 3330. The matrix 2630 may be supported on a substrate comprising the one more liners 3320 or the one or more adhesives 3330. The matrix 2630 can comprise any combination of one or more matrices 3310, one or more liners 3320, or one or more adhesives 3330. For example, the matrix 2630 can comprise a matrix, a liner, and/or an adhesive. For example, the matrix 2630 can comprise a matrix. The matrix 3310 can be configured to yield quality plasma using, for example, a treated or an untreated glass fiber matrix material. The liner 3320 can be configured to separate the adhesive 3330 from the matrix 3310 to support a thin matrix 3310 while under gravitational and/or liquid blood weight loads. For example, the matrix 3310 may be adhered at one or more edges so the liner 3320 may prevent the matrix 3310 from separating from the adhesive 3330. The adhesive 330 can comprise an adhesive mylar material, an adhesive inert, a biocompatible material, and the like.

The liner 3320 may extend completely between the substrate and the matrix 2630. The liner may extend between the substrate and the matrix 2630 in a first region and does not extend between the substrate and the matrix 2630 in a second region that is different from the first region. The first region may comprise a central portion of the matrix 2630, and the second region may comprise one or more end portions of the matrix 2630.

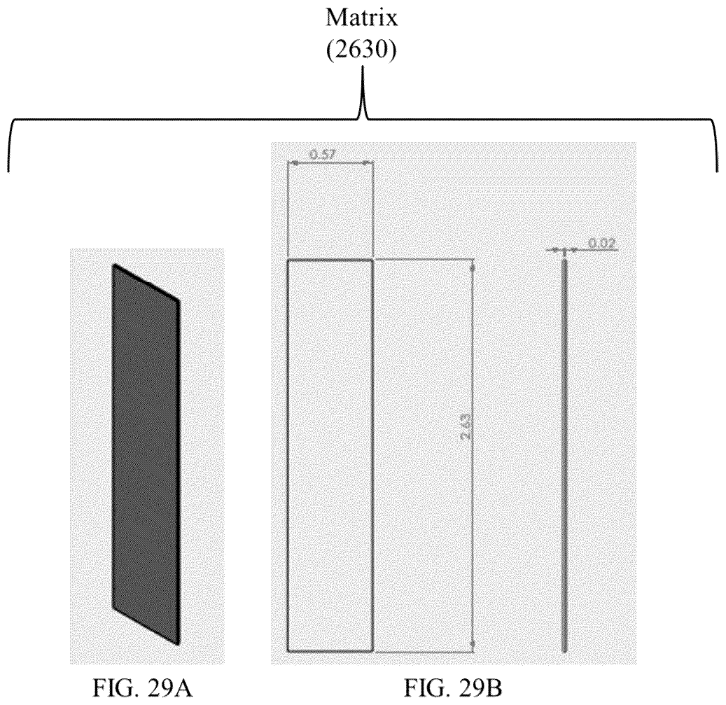

FIGS. 29A-29B illustrate nonlimiting dimensions of the treatment/stabilization unit 2630 (e.g., a matrix described elsewhere herein). For example, as shown in FIG. 29B, the matrix 2630 may have a length ranging from about 1.3 inches to about 4.0 inches. The length may be measured from an end of the matrix 2630 to an opposite end of the matrix 2630. In some preferred embodiments, the length may be about 2.1 inches to about 3.2 inches. The matrix 2630 may have a width ranging from about 0.3 inches to about 0.9 inches. The width may be measured from a side of the matrix 2630 to an opposite side of the matrix 2630. In some preferred embodiments, the width may be about 0.5 inches to about 0.7 inches. The matrix 2630 may have a thickness ranging from about 0.01 inches to about 0.03 inches. The thickness may be measured from a surface of the matrix 2630 to an opposite surface of the matrix 2630. In some preferred embodiments, the thickness may about 0.016 inches to about 0.024 inches. In some cases, the thickness may comprise a thickness of the matrix 3310, the liner 3320, and the adhesive 3330. In some cases, the thickness may comprise a thickness of any combination of the matrix 3310, the liner 3320, or the adhesive 3330. The matrix 3310 may have a surface area ranging from about 0.75 in² to about 2.25 in². In some preferred embodiments, the surface area may be about 1.2 in² to about 1.8 in². The matrix 3310 may have a length to width ratio ranging from about 2.3 to about 7.0. In some preferred embodiments, the length to width ratio may be about 3.7 to about 5.5.

The matrix 3310 may be located a distance from a distal end of the cartridge port 2620 to a proximal end of the matrix having a range of about 0 mm to about 15 mm. In some preferred embodiments, the distance may be about 10 mm. The matrix 3310 may be located a distance from a distal end of the cartridge port 2620 to a distal end of the matrix 2630 having a range of about 35 mm to about 115 mm. In some preferred embodiments, the distance may be about 75 mm. An edge of the matrix 3310 may be configured to extend to and substantially align with the pressure bar 3040. An edge of the matrix 3310 may be located a distance from the pressure bar 3040 ranging from about 0 mm to about 10 mm. An edge of the matrix 3310 may extend a distance beyond the pressure bar 3040 towards the reservoir 3030 ranging from about 0 mm to about 10 mm. An edge of the matrix 3330 may extend a distance beyond the pressure bar 3040 and into the reservoir 3030 ranging from about 0 mm to about 10 mm. An edge of the matrix 330 may be configured to not extend beyond the pressure bar 3040 into the reservoir 3030.

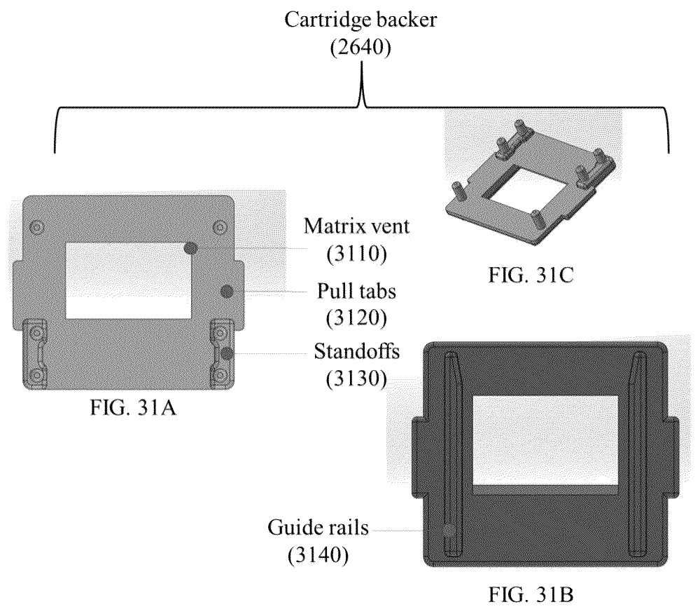

FIGS. 31A-31C illustrate a nonlimiting embodiment of the cartridge backer 2640. The cartridge backer 2640 can comprise one or more features. For example, the cartridge backer 2640 can comprise one or more matrix vents 3110, one or more pull tabs 3120, one or more standoffs 3130, or one or more guide rails 3140. The one or more matrix vents 3110 may be configured with a vacuum to provide, for example, better quality plasma yield than a cartridge assembly without a vacuum. A number of vents may comprise about 1, 2, 3, or more vents. The one or more pull tabs 3120 may be configured to aid, for example, assembly, disassembly, or other processing operations. A number of pull tabs may comprise about 1, 2, 3, or more pull tabs. The standoffs 3130 may be configured to regulate, for example, pressure for the pressure bar 3040. The standoffs 3130 may be configured to create a gap between the cartridge port 2620 and the cartridge backer 2640. The gap may be configured to be used in part with the pressure bar 3040 to regulate a flow speed of the blood sample and ensure proper wicking of the blood sample along the matrix 2630. A number of standoffs may comprise about 1, 2, 3, or more standoffs. The guide rails 3140 may be configured to allow, for example, a user to install the cartridge assembly 2600 into the sample acquisition device 1100. The guide rails 3130 may comprise a pair of guide rails that are laterally spaced apart on the cartridge backer (e.g., the backer plate) 2640. A number of guiderails may comprise about 1, 2, 3, or more guide rails.

Referring again to FIGS. 28A-28E, the cartridge backer (e.g., a backing plate) 2640 may be configured to operatively couple to the cartridge port 2620. The operatively coupling of the cartridge backer 2640 to the cartridge port 2620 may be illustrated here using nonlimiting embodiments. For example, the cartridge backer 2640 may include features of one or more standoffs (e.g., spacers) 3130 that are sized and aligned to be received by and secured to the cartridge port 2620. Any method may be used to secure the cartridge backer 2640 to the cartridge port 2620. Nonlimiting examples of securing the cartridge backer 2640 may include mechanical methods (e.g., screws, rivets, tabs, etc.) or any other method (e.g., adhesives, pressure fittings, etc.). For example, the cartridge backer 2640 may comprise features of one or more guide rails 3140 that are configured to be sized and aligned to be received by and secured to the cartridge port 2620. For example, the cartridge backer 2640 may include features of one or more pull tabs 3140 that are configured to be sized and aligned to be received by and secured to the cartridge port 2620. The cartridge backer 2640 may have a length ranging from about 0.6 inches to about 1.8 inches. The length may be measure from an end of the cartridge backer 2640 to an opposite end of the cartridge backer 2640. In some preferred embodiments, the length may be about 1.0 inches to about 1.5 inches. The cartridge backer 2640 may have a width ranging from about 0.5 inches to about 1.4 inches. The width may be measure from a side of the cartridge backer 2640 to an opposite side of the cartridge backer 2640. In some preferred embodiments, the width may be about 0.75 inches to about 1.1 inches. The cartridge backer 2640 may have a thickness ranging from about 0.05 inches to about 0.15 inches. In some preferred embodiments, the thickness may be about 0.08 inches to about 0.12 inches. The cartridge backer 2640 may have a surface area ranging from about 0.6 in² to about 1.7 in². In some preferred embodiments, the surface area may be about 0.9 in² to about 1.4 in². The cartridge backer 2640 may have a length to width ratio ranging from about 0.7 to 2.0. In some preferred embodiments, the length to width ratio may be about 1.1 to 1.6. The length to width ratio of the cartridge backer 2640 may be configured to and compatible with a matrix 2630 having a length to width ratio to yield quality plasma.

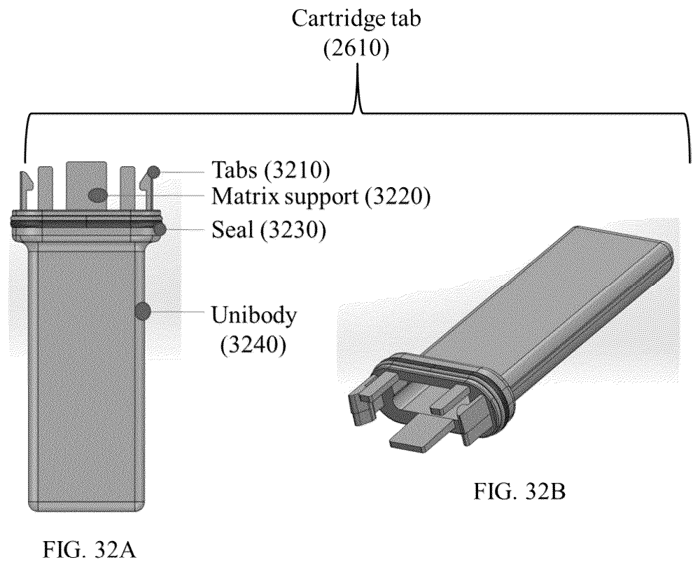

FIGS. 32A-32B illustrate a nonlimiting embodiment of the cartridge tab 2610. The cartridge tab (e.g., the enclosure) 2610 may be fully enclosed. The cartridge tab 2610 can comprise one or more features. For example, the cartridge tab 2610 can comprise on or more tabs 3210, one or more matrix supports 3220, one or more elongated seals 3230, or a unibody 3240. The one or more tabs 3210 may be configured to, for example, secure the cartridge tab 2610 to the cartridge port 2620, the matrix 2630, or the cartridge backer 2640. The matrix support 3220 may be configured to, for example, secure the matrix 2630 to the cartridge assembly 2600. The seal (e.g., vent seal) 3230 may be configured to, for example, ensure a vacuum flows freely through the matrix 2630, into the reservoir 3030, and throughout elongated seal 2650 to maintain system continuity (e.g., flow of blood through the matrix). The vent seal 3230 may be configured to permit vacuum pressure to equalize throughout and within and the cartridge assembly 2600. The unibody 3240 may be configured to, for example, ensure a vacuum seal and secure the matrix 2630 to the cartridge assembly 2600. The unibody 3240 may be configured to permit vacuum pressure to equalize throughout and within and the cartridge assembly 2600. The elongated seal 2650 may extend along an opening of the cartridge tab 2610 to hermetically seal the enclosure of the cartridge tab (e.g., elongated housing) 2610.

Referring again to FIGS. 27A-27C, FIGS. 27A-27C illustrate nonlimiting dimensions of the elongated housing (e.g., the cartridge tab) 2610. For example, as shown in FIG. 27B, the cartridge tab 2610 may have a length ranging from about 0.9 inches to about 2.8 inches. The length may be measure from an end of the cartridge tab 2610 to an opposite end of the cartridge tab 2610. In some preferred embodiments, the length be about 1.5 inches to about 2.2 inches. The cartridge tab 2610 may have a first width ranging from about 0.5 inches to about 1.4 inches. The first width may be measure at an end of the cartridge tab 2610 having the elongated seal 2650 and from a side to an opposite side of the cartridge tab 2610. In some preferred embodiments, the first width may be about 0.75 inches to about 1.0 inches. The cartridge tab 2610 may have a second width ranging from about 0.4 inches to about 1.1 inches. The second width may be measure at an opposite end of the cartridge tab 2610 from the elongated seal 2650 and from a side to an opposite side of the cartridge tab 2610. In some preferred embodiments, the second width may be about 0.6 inches to about 0.9 inches. The cartridge tab 2610 may have a first thickness of about 0.2 inches to about 0.7 inches. The first thickness may be measured from a side of the elongated seal 2650 to an opposite side of the elongated seal 2650. In some preferred embodiments, the first thickness may be about 0.40 inches to about 0.60 inches. The cartridge tab 2610 may have a second thickness of about 0.18 inches to about 0.53 inches. The second thickness may be measured from a side of the cartridge 2610 to an opposite side of the cartridge tab 2610. In some preferred embodiments, the second thickness may be about 0.3 inches to about 0.40 inches. The cartridge tab 2610 may have a surface area ranging from about 0.7 in² to about 2.5 in². In some preferred embodiments, the surface area may be about 1.0 in² to about 2.0 in². The cartridge tab 2610 may have a length to width ratio ranging from about 1.0 to about 3.8. In some preferred embodiments, the length to width ratio may be about 1.6 to about 2.4. The length to width ratio of the cartridge tab 2610 may be configured to and compatible with a matrix 2630 having a length to width ratio to yield quality plasma.

FIGS. 34A-34D illustrate operation of the cartridge assembly 2600 which can be used with the sample acquisition device 1100. The sample acquisition device 1100 may comprise one or more flow indicators 170 described elsewhere herein that are configured to measure or view flow of blood during sample acquisition through the indication window 3050 of the cartridge assembly 2600. FIG. 34A illustrates that, before sample acquisition, the sample (e.g., blood) may not be visible on the matrix 2630 as viewed through the indication window 3050. FIGS. 34B and 34C illustrate that, during sample acquisition, the sample (e.g., blood) may be visible on the matrix 2630 as viewed through the indication window 3050. Small aberrations in the flow field of the blood on the matrix 2630 may be normal. FIG. 34D illustrates that, at completion of sample acquisition (e.g., blood), blood acquisition may be complete when the width of the matrix 2630 has blood as viewed through the indication window 3050. Sample acquisition may result in blood appearing uniformly or nonuniformly as viewed through the indication window 3050. Sample acquisition may be complete when 1) the indication window 3050 is viewed as substantially filled with blood, 2) the blood is starting to become visible through the indication window 3050, or 3) about 10 minutes have elapsed from the start of sample acquisition (e.g., blood), or any combination thereof. The plasma of the sample may continue to normalize the flow field of the sample for up to about 5 minutes, 10 minutes, 20 minutes, or more after the start of sample acquisition (e.g., blood).

Operation of the cartridge assembly 2600 may be configured to use different combinations of treatments and/or agents described elsewhere herein. For example, treatments may be added to the materials or features of the cartridge assembly 2160, to the matrix 2630, to the sample (e.g., blood), or any combination thereof to make it easier to detect and/or inspect the plasma regions of the matrix 2630 visually (e.g., optical detection, ultraviolet (UV) detection, and/or infrared (IR) detection). The plasma regions having treatments may be detected and/or inspected by users and/or instruments (e.g., optical/visible detectors, UV detectors, or IR detectors). The treatments may be configured to optimize for plasma separation having different analytes to be detected, inspected, or measured. The treatments may be configured to inform a user when enough plasma has been collected. The treatments may be configured to stabilize a whole blood region and/or a plasma region of the matrix 2630 for analyte recovery. Treatments and/or agents may include chemical treatments, sugar treatments, surfactant treatments, or any combination thereof. Treatments may be configured for: a user experience by indicating an ideal time to remove the sample acquisition device 1110; a lab technician to improve throughput efficiencies for recovering analytes; generation of more accurate results by analyzing plasma quality with high quality yield; a user, lab technician, or machine to provide visual indications of plasma quality and yield; software and/or hardware solutions designed to work in corroboration with the sample acquisition device 1100 for pre-processing, pen-processing, and/or post-processing and analysis of the plasma separation matrix.

Plasma quality (e.g., blood plasma separation performance) may be assessed using, for example, hemolysis of the sample (e.g., blood) within the blood and/or the plasma of the cartridge assembly 2600. Hemolysis may have a percentage ranging from about 0% to about 25% or more. In some preferred embodiments, hemolysis may be about 5%. Blood plasma separation performance may be improved, using features described herein, as compared to a use without the features, for example, a pressure bar 3040, a matrix having a determined length to width ratio, a seal vent 2650, a matrix vent 3060, or any combination thereof. For example, blood plasma separation performance of the matrix may be improved by at least about 5% with use of the pressure bar 3040 compared to without the use of the pressure bar 3040. For example, blood plasma separation performance of the matrix may be improved by at least about 5% when a length of the elongated strip 2630 is at least about 4.7 times greater than a width of the elongated strip 2630. For example, blood plasma separation performance of the matrix may be improved by at least about 5% with use of the seal vent 2650 compared to without the use of the seal vent 2650. For example, blood plasma separation performance of the matrix is improved by at least about 5% with use of the matrix vent 3060 compared to without the use of the matrix vent 3060.

As used in the specification and claims, the singular form “a”, “an” and “the” include plural references unless the context clearly dictates otherwise. For example, the term “a cell” includes a plurality of cells, including mixtures thereof.

As used herein, the term “about” a number refers to that number plus or minus 10%, 9%, 8%, 7%, 6%, 5%, 4%, 3%, 2%, 1%, of that number.

As used herein, and unless otherwise specified, the term “substantially” and similar terms are defined as largely but not necessarily wholly what is specified, as understood by a person of ordinary skill in the art. In any disclosed embodiment, the terms “substantially,” “approximately,” “generally,” and “about” may be substituted with “within [a percentage] of” what is specified, where the percentage includes 0.1, 1, 5, and 10 percent.

### EXAMPLES

### Example 1: Cartridge Assembly for Blood Separation

FIG. 3C, FIG. 3F, and FIG. 4 show various examples of a cartridge assembly for separating plasma or serum from blood collected from a subject. The cartridge assembly can be coupled to and in fluid communication with a sample acquisition device (e.g., the sample acquisition device 100, as shown in FIG. 3D) to receive the blood from the subject. The cartridge assembly can comprise a port to provide a pathway for the fluid communication between the cartridge assembly and the sample acquisition device. The cartridge assembly can comprise one or more blood treatment/stabilization units to separate the plasma or serum from the blood. A blood treatment/stabilization unit can be a stack of multiple components (or layers). For example, a blood treatment/stabilization unit can comprise multiple layers, e.g., (1) a pre-filter layer to filter out cells and/or debris from the blood, (2) a blood separation membrane to isolate the serum or plasma from the remnants of the blood that is passed through the pre-filter, and (3) a collection media to collect and/or store the isolated serum or plasma.

As shown in FIGS. 3C and 3F, a direction of flow of the blood through at least a portion of the pathway of the cartridge assembly's port can be different from a direction of flow of the blood through the blood treatment/stabilization unit(s). Referring to FIG. 3C, the pathway 340 of the port 330 of the cartridge assembly 300 can comprise (i) a proximal end in fluid communication with the sample acquisition device and (ii) a distal end in fluid communication with the blood treatment/stabilization unit(s) 320. The pathway 340 can direct the blood to flow from the sample acquisition device into the proximal end in a first direction, through the pathway 340, and exit from the distal end onto the blood treatment/stabilization unit(s) 320 in a second direction that is different from the first direction. An angle of intersection between the first direction and the second direction can be greater than zero degree and less than 180 degrees. The direction of flow of blood through the blood treatment/stabilization unit(s) 320 can be substantially orthogonal to a longitudinal axis 346 of the cartridge assembly 300. Referring to FIG. 4F, the pathway 340 of the port 330 of the cartridge assembly 300b can be substantially parallel to a longitudinal axis 346 of the cartridge assembly 300b, and the direction of flow of blood through the blood treatment/stabilization unit(s) 320 can be substantially orthogonal to the longitudinal axis 346 of the cartridge assembly 300b. In addition, the cartridge assembly 300b can comprise a collection reservoir 362 configured to contain the blood collected from the sample acquisition device prior to or during the plasma or serum separation by the blood treatment/stabilization unit(s) 320.

Referring to FIG. 4, the pathway 440 of the port 410 of the cartridge assembly 400 can direct the blood to flow from the sample acquisition device into a proximal end of the blood treatment/stabilization units 420a, 420b in a direction that is substantially the same as a direction of flow of blood through the blood treatment/stabilization units 420a, 420b.

### Example 2: Cartridge Assembly for Storing Liquid Blood

FIG. 5A shows an example cartridge assembly 500 for storing a liquid sample, such as liquid blood. The cartridge assembly 500 can comprise a coupling unit 510 configured to couple to a cartridge chamber of a sample acquisition device (e.g., the sample acquisition device 100, as shown in FIG. 5B) configured to collect the liquid blood from a subject. The cartridge assembly 500 can comprise a container 520 configured to store the liquid blood. The cartridge assembly 500 can comprise a cartridge holder 540 configured to support the container 520. A proximal end of the container 520 can be configured to couple to the coupling unit 510, and a distal end of the container 520 can be configured to couple to the cartridge holder 540. The coupling unit 510 can comprise one or more fluidic pathways 516. As illustrated in FIG. 5B, the container 520 can be configured to receive the liquid blood flowing into the container 520 in a first direction 524. The one or more fluidic pathways 516 can be configured to direct and expunge the air out of the container 520 in a second direction 526 that is different from the first direction 524.

### Example 3: Modular Chamber Assembly for Storing Blood in a Plurality of Different Formats

FIG. 7A shows an example modular chamber assembly 600 for storing blood collected from a subject in a plurality of different formats selected from the group consisting of: plasma, serum, dried blood, liquid blood, and coagulated blood. The modular chamber assembly 600 can comprise an inlet port 610 (e.g., a pierceable self-sealing cap) that can be removable from the rest of the modular chamber assembly 600. The modular chamber assembly 600 can comprise a chamber 620 that comprises a cartridge assembly 630. The cartridge assembly 630 can include one of a plurality of different cartridge assembly types that permit the blood to be collected, processed, or stored in the plurality of different formats. For example, the cartridge assembly 630 can comprise a cartridge 640 that comprises one or more matrix strips 642 to absorb and collect the blood or a portion thereof from the subject. The cartridge 640 can also comprise one or more absorbent pads 644 for holding and metering out excess blood.

As illustrated in FIG. 8A, the modular chamber assembly 600 can be operatively coupled to a modular sample acquisition device 900b to collect the blood from the subject.

### Example 4: Recovery Rates of Analytes in a Blood Sample

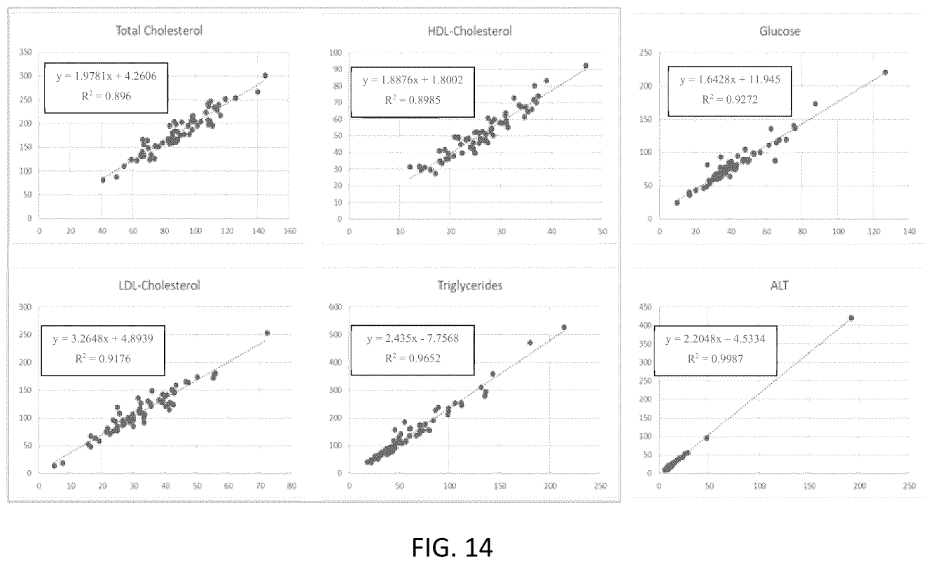

FIG. 14 illustrates the linear regression analyses performed on the data from studies measuring the recovery rates of several analytes after the separation of a blood sample collected from a blood separation assembly as described herein. The analytes tested include: total cholesterol, HDL-cholesterol, LDL-cholesterol, Triglycerides, ALT, and Glucose.

The test included, first, introducing 225 μL of a blood sample into a blood separation assembly. The samples were allowed to dry overnight, and the analytes were eluted from the collection matrix in the blood separation assembly. The eluted samples were tested on a Beckman Coulter AU480 analyzer with Beckman Coulter reagents. 66 independent samples were tested under these constant protocols.

The R² value for the recovery rate each of the analytes is shown in FIG. 14. The y-axis in each graph represents the amount of analyte in a plasma sample received from a donor. The x-axis in each graph represents the amount of analyte recovered in an eluted sample, with adjustments made for the hematocrit level of the plasma donor. The results are summarized in Table 1.

TABLE 1
Linear Regression Analysis on Analyte Recovery

| Analytes | R² Value | Recovery Rate |
|---|---|---|
| Total Cholesterol | .896 | 50% |
| HDL-cholesterol | .898 | 51% |
| LDL-cholesterol | .918 | 30% |
| Triglycerides | .965 | 45% |
| ALT | .999 | 57% |
| Glucose | .927 | 52% |

### Example 5: Collection Matrix Elution Protocol

As described herein, elution methods may be required to successfully recover a separated blood sample from a collection matrix. Required materials for an exemplary elution method include: tweezers, a cutting mat, razor blades or a scalpel with replaceable blades, 1-2 mL tubes, PBS buffer solution, Tween-80 solution, and an orbital shaker suitable for 1-2 mL tubes.

The first step in the elution method allows for a sample to dry in the collection matrix inside of a cartridge assembly overnight. Once dry, the multi-piece collection matrix can be removed from the rest of the treatment/stabilization unit using tweezers. The bottom piece of the multi-piece collection matrix can then be separated from the top piece of the collection matrix either by pulling or cutting the bottom piece away from the top piece of the collection matrix. Using a scalpel/razor blade, two perpendicular cuts can be made on the bottom piece of the collection matrix to create four equal pieces. The dimensions of the four pieces can all be about 7.5×6 mm each, allowing for the four smaller pieces to fit in a micro tube. Once the four pieces are placed in the micro tube, 225 mL of 10 mM PBS buffer with 0.02% Tween-80 can be added. The micro tube can be quick-spun to ensure no droplets remain on the walls. In an optimal elution, the liquid should cover at least 40% of the four smaller pieces of the bottom piece of the collection matrix. The micro tube can then be placed in an orbital shaker, where it can shake for 1 hour at room temperature at 850 rpm. Once the shaking is complete, the four pieces will have uniform color and at least 100-120 mL of elute volume can be recovered and used to analyze the properties of the sample recovered in the collection matrix.

### Example 6: Matrix Dimensions and Ratios Studies

Matrices with dimensions as provided in Table 2 were generated. All matrices were treated with 250 μL of blood and the results were measured in terms of blood length (length of the matrix with red blood cells), plasma length (length of the matrix with plasma) and the plasma area. Table 2 shows that different width/length ratios result in different useable amounts of plasma area. These width to length ratios are important for some given dynamic range of sample volume. In some cases, the width to length ratios may be optimized for sample volumes that range from about 150 μL to about 250 μL or more.

TABLE 2
Matrix dimensions, ratios, and blood collection results

| | | | | | Blood | Plasma | Length: | Plasma | Actual |
|---|---|---|---|---|---|---|---|---|---|
| | Width | Length | Area | | length | Length | Plasma | area | Sample |
| Sample | (in) | (in) | (in²) | L:W | (in) | (in) | length | (in²) | (mg) |
| 1 | 0.63 | 2.5 | 1.56 | 4.0 | 1.299 | 0.787 | 0.315 | 0.492 | 120 |
| 2 | 0.63 | 2.5 | 1.56 | 4.0 | 1.299 | 0.787 | 0.315 | 0.492 | 120 |
| 3 | 0.67 | 3.35 | 2.24 | 5.0 | — | — | — | — | — |
| 4 | 0.71 | 2.12 | 1.5 | 3.0 | 0.740 | 0.660 | 0.311 | 0.467 | 108 |
| 5 | 0.61 | 2.45 | 1.5 | 4.0 | 1.220 | 1.500 | 0.612 | 0.919 | 140 |
| 6 | 0.55 | 2.74 | 1.5 | 5.0 | — | — | — | — | — |
| 7 | 0.5 | 3 | 1.5 | 6.0 | 1.200 | 1.080 | 0.360 | 0.540 | 138 |
| 8 | 0.46 | 3.24 | 1.5 | 7.0 | 1.496 | 0.787 | 0.243 | 0.364 | 140 |

As shown in the Table 2, increasing the length of the matrix beyond a certain threshold value may not necessarily lead to an increase in plasma collection. For instance, row 8 (super narrow) shows that the long tail of the membrane does not increase the yield of plasma. However, a shorter material at this same width would over saturate the membrane and yield no plasma. Therefore, an ideal length to width ratio is important for sample recovery. For instance, for this sample volume, the ideal length:width ratio may be between about 3 to 5. This ratio may be the ratio that is flexible to various sample volumes, hematocrit levels, large plasma region, and a higher concentration of plasma.

Several examples of membranes with different length:width ratios are provided in FIGS. 23A-F. FIG. 23A illustrates a thin, narrow, sample of LF1 membrane which shows an oversaturation event where no clean plasma can be sampled. FIG. 23B illustrates a thick MF1 membrane material shows an undersaturation event where on a small plasma region is available. FIG. 23C illustrates an undersaturated wide material where blood is absorbed and no plasma is available. FIG. 23D illustrates an exemplary optimal geometry where close to a 50/50 ratio of whole blood/plasma is observed. FIG. 23E illustrates an example where the matrix is much longer but is still within the “ideal” range where a good plasma yield is observed. FIG. 23F illustrates an example of a long matrix that yields good plasma.

### Example 7: Geometric Features and Treatments

Several examples of pretreatment of a matrix and their plasma collection results are provided in FIGS. 25A-D. As shown in FIG. 25A, a reagent may be used to pretreat the matrix material to help fluoresce the plasma region in a visible wavelength of light. The reagent may comprise any reagent as described herein. In FIG. 25B a UV light was used to more visible see the plasma region in a matrix. FIG. 25C illustrates another example of how the plasma region (in a matrix with a different geometric configuration) is more easily observed using a UV light. In FIG. 25D, a pre-treatment was used to better demarcate the plasma region. The top image of FIG. 25D shows a poor whole blood to plasma transfer region, when the bottom one shows a more sharply defined break.

### Example 8: Plasma Quality and Extraction Studies of HbA1c and Lipid Profiles

A length to width ratio of matrix may be optimized to yield quality plasma separation according to: a given matrix glass fiber material (e.g., Cytiva® LF1), a range of sample volumes (e.g., volume of blood sample), a range of hematocrit levels, or a thickness of a matrix. Optimizing a length to width ratio of a matrix may beneficially optimize: plasma volume yield per surface area, a larger plasma to whole blood surface area, plasma spread and extraction across a larger area for a great number of biomarkers that can be analyzed, insensitivity to sample volumes, insensitivity to hematocrit levels, user experience due to less sample acquisition time, ease of manufacturing, or less destruction of red blood cells.

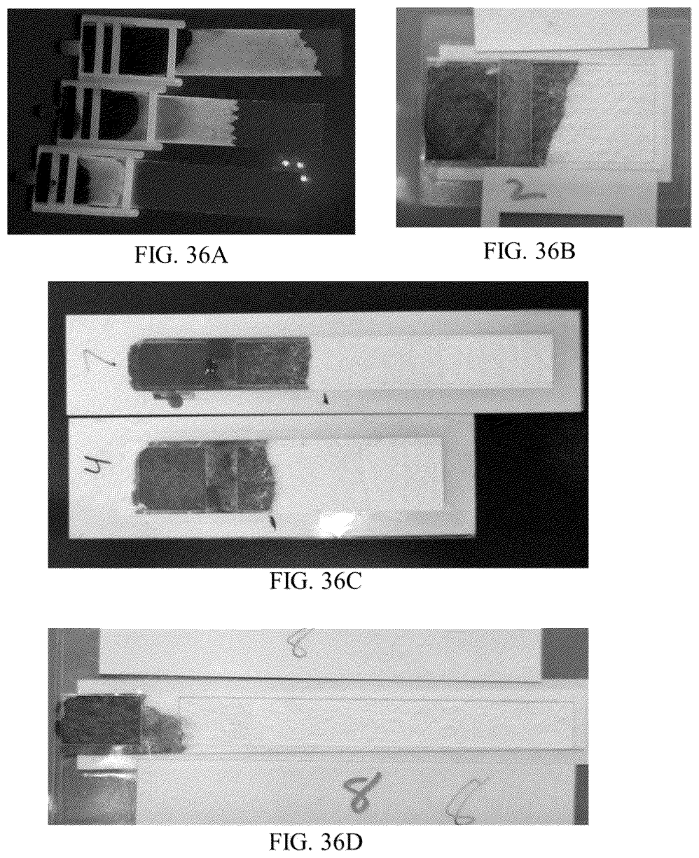

Matrices having different ratios of length to width were generated to assess plasma yield and quality. All matrices used the same glass fiber filter material (e.g., Cytiva® LF1) and were treated with 175 μL of EDTA donor blood. The results were measured in terms of plasma yield assessed by a length of the matrix with plasma. The plasma yield may be visualized as shown in FIGS. 36A-36D. Table 3 shows that a ratio of about 3 to 6 results in a plasma yield quality score of about 4 or 5. A ratio of about 3 to 6 may be useful for yielding high quality plasma. Also, Table 3 may show that there is a threshold matrix length at which matrix length may not improve quality plasma and yield. For example, a threshold matrix length may exist where plasma does not arrive to a portion of the matrix beyond the threshold matrix length.

TABLE 3
Plasma quality scores with different matrix ratios

| | | | | Result | Result | |
|---|---|---|---|---|---|---|
| | Matrix | Matrix | Ratio of | Blood | Plasma | Plasma |
| | Width | Length | length to | Length | Length | Quality |
| Sample | (in) | (in) | width | (in) | (in) | (1-5) |
| 1 | 0.71 | 2.12 | 2.99 | 0.740 | 0.660 | 3 |
| 2 | 0.71 | 2.12 | 2.99 | 0.780 | 0.660 | 3 |
| 3 | 0.71 | 2.12 | 2.99 | 0.640 | 0.660 | 3 |
| 4 | 0.63 | 2.50 | 3.97 | 1.299 | 0.787 | 5 |
| 5 | 0.63 | 2.50 | 3.97 | 1.254 | 0.812 | 5 |
| 6 | 0.63 | 2.50 | 3.97 | 1.177 | 0.912 | 5 |
| 7 | 0.55 | 2.74 | 4.98 | 1.376 | 0.654 | 4 |
| 8 | 0.55 | 2.74 | 4.98 | 1.322 | 0.665 | 4 |
| 9 | 0.55 | 2.74 | 4.98 | 1.401 | 0.621 | 4 |
| 10 | 0.50 | 3.00 | 6.00 | 1.278 | 0.864 | 5 |
| 11 | 0.50 | 3.00 | 6.00 | 1.187 | 0.811 | 5 |
| 12 | 0.50 | 3.00 | 6.00 | 1.154 | 0.832 | 4 |

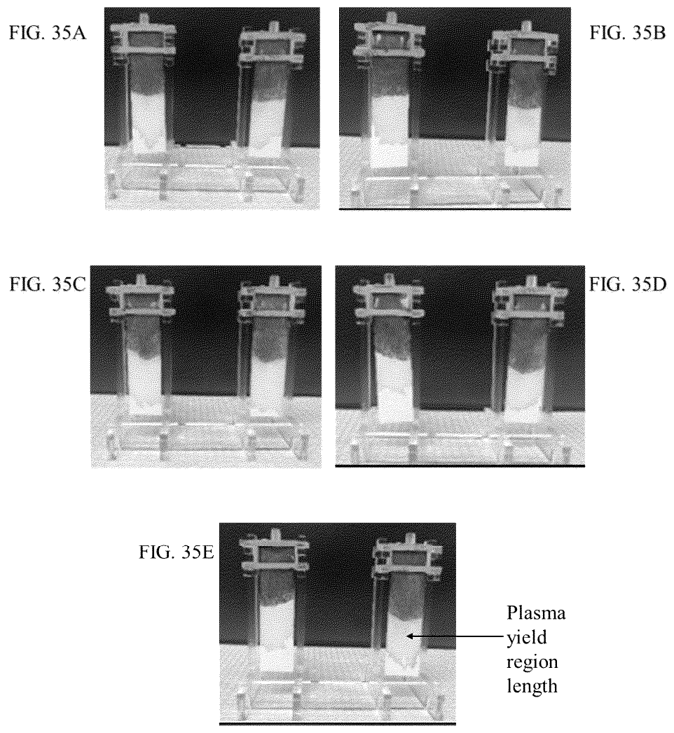

Matrices having a same ratio of length to width (ratio of 4.7 resulting from 2.66 inches in length and 0.57 inches in width) were generated to assess plasma yield and quality. All matrices used the same glass fiber filter material (e.g., Cytiva® LF1). Matrices were treated with 175 μL of EDTA donor blood from ten samples. The results were measured in terms of plasma yield (length of the matrix with plasma) to determine a ratio of plasma yield to length of the matrix strip. The plasma yield may be visualized as shown in FIGS. 35A-35E. FIG. 35A corresponds to samples 1 and 2, FIG. 35B to samples 3 and 4, FIG. 35C to samples 5 and 6, FIG. 35D to samples 7 and 8, and FIG. 35E to samples 9 and 10. Table 4 shows that a ratio of about 4.7 results in a plasma yield quality score of about 4 to 5. A ratio of about 4.7 may be useful for yielding high quality plasma.

TABLE 4
Plasma quality scores

| | | | | Plasma | | |
|---|---|---|---|---|---|---|
| | | | | Yield | | Plasma |
| | | | | Region | Plasma | Quality |
| | Blood | Matrix | Matrix | Length | to Strip | Score |
| Sample | Sample | Material | Sample | (in) | Length | (1-5) |
| 1 | SP1 (EDTA) | Glass-fiber | LF1 (1) | 0.9 | 0.338 | 5 |
| 2 | SP1 (EDTA) | Glass-fiber | LF1 (2) | 0.81 | 0.304 | 5 |
| 3 | SP2 (EDTA) | Glass-fiber | LF1 (3) | 0.87 | 0.327 | 5 |
| 4 | SP2 (EDTA) | Glass-fiber | LF1 (4) | 0.84 | 0.316 | 5 |
| 5 | SP3 (EDTA) | Glass-fiber | LF1 (5) | 1.05 | 0.395 | 4 |
| 6 | SP3 (EDTA) | Glass-fiber | LF1 (6) | 0.98 | 0.368 | 4 |
| 7 | SP4 (EDTA) | Glass-fiber | LF1 (7) | 0.69 | 0.259 | 5 |
| 8 | SP4 (EDTA) | Glass-fiber | LF1 (8) | 0.72 | 0.271 | 5 |
| 9 | SP5 (EDTA) | Glass-fiber | LF1 (9) | 0.81 | 0.304 | 4 |
| 10 | SP5 (EDTA) | Glass-fiber | LF1 (10) | 0.85 | 0.319 | 4 |

The matrices and samples of Table 4 were further assessed to validate the extraction and recovery of HbA1c from 175 μL of EDTA donor blood. The results were measured in terms of the percent recovery of HbA1c and compared to controls C1 and C2. Table 5 compares the percent recovery of HbA1c to the expected recovery and a target. The percent recovery of HbA1c may be useful for qualifying a cartridge assembly having the matrices for A1C.

TABLE 5
Percent recovery of HbA1c

| | | HbA1c | HbA1c | HbA1c | |
|---|---|---|---|---|---|
| | | Expected | Expected | Observed | Recovery |
| ID | Target | (%) | (mmol/mol) | (mmol/mol) | (%) |
| HbA1c C1 | <6% | 5.6 | 37.7 | 15.5 | 41.11% |
| HbA1c C2 | >10% | 11.2 | 98.9 | 13 | 13.14% |
| S1-1 | <6% | 5.6 | 37.7 | 18 | 47.74% |
| S2-1 | 6-7% | 6 | 42.1 | 11.6 | 27.57% |
| S3-1 | 7-8% | 7.6 | 59.6 | 14.4 | 24.18% |
| S4-1 | 8-9% | 8.3 | 67.2 | 15.8 | 23.51% |
| S5-1 | 9-10% | 9.5 | 80.3 | 11.2 | 13.94% |
| S6-1 | >10% | 11.2 | 98.9 | 10.5 | 10.62% |
| S3-2 | 7-8% | 7.6 | 59.6 | 14.5 | 23.34% |
| S4-2 | 8-9% | 8.3 | 67.2 | 14.8 | 22.02% |
| S5-2 | 9-10% | 9.5 | 80.3 | 10.3 | 12.82% |
| S6-2 | >10% | 11.2 | 98.9 | 10.1 | 10.21% |

The matrices and samples of Table 4 were further assessed to validate the extraction of lipid profiles from 175 of EDTA donor blood. Lipids included cholesterol (total), cholesterol (HDL), cholesterol (LDL), and triglycerides. The results were measured in terms of a percent recovery of lipids and compared to plasma controls. Plasma controls were configured with or spiked with known concentrations of lipids. Table 6 illustrates the percent recovery of lipids. The percent recovery of lipid profiles may be useful for qualifying the cartridge assembly having the matrices for lipid profiles.

TABLE 6
Percent recovery of lipid profiles

| | | | Endogenous | | | | |
|---|---|---|---|---|---|---|---|
| | | | Value | Spike | Expected | Observed | Recovery |
| Sample | ID | Target | (mg/dL) | Controls | (mg/dL) | (mg/dL) | (%) |
| 24 | Plasma | Cholesterol (Total) | 235 | | 235 | 235 | |
| | Control | Cholesterol (HDL) | 37 | | 37 | 37 | |
| | | Cholesterol (LDL) | 145 | | 145 | 145 | |
| | | Triglycerides | 264 | | 264 | 264 | |
| 15 | L1 | Cholesterol (Total) | 164 | | 164 | 13 | 8.0% |
| | Extract | Cholesterol (HDL) | 25 | | 25 | 4 | 16.2% |
| | Control | Cholesterol (LDL) | 117 | | 117 | 8 | 6.4% |
| | | Triglycerides | 110 | | 110 | 8 | 7.3% |
| 16 | L2 | Cholesterol (Total) | 320 | | 320 | 23 | 7.2% |
| | Extract | Cholesterol (HDL) | 55 | | 55 | 9 | 16.8% |
| | Control | Cholesterol (LDL) | 184 | | 184 | 7 | 4.0% |
| | | Triglycerides | 402 | | 402 | 32 | 7.8% |
| 13 | LN1 - | Cholesterol (Total) | 235 | 164 | 200 | 13 | 6.5% |
| | Spike | Cholesterol (HDL) | 37 | 25 | 31 | 5 | 14.8% |
| | Extract | Cholesterol (LDL) | 145 | 117 | 131 | 6 | 4.7% |
| | | Triglycerides | 264 | 110 | 187 | 11 | 5.9% |
| 14 | LN2 - | Cholesterol (Total) | 235 | 320 | 278 | 18 | 6.5% |
| | Spike | Cholesterol (HDL) | 37 | 55 | 46 | 7 | 15.8% |
| | Extract | Cholesterol (LDL) | 145 | 184 | 165 | 6 | 3.7% |
| | | Triglycerides | 264 | 402 | 333 | 24 | 7.1% |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1.-20. (canceled)

21. A cartridge assembly comprising:
an inlet component comprising a port configured to receive a blood sample;
an elongated strip comprising a matrix configured to separate and collect plasma from the blood sample;
a backing plate configured to couple to the inlet component and secure a proximal portion of the elongated strip between the inlet component and the backing plate; and
an elongated housing configured to releasably couple to the inlet component, the elongated housing comprising an enclosure for receiving the elongated strip.

22. The cartridge assembly of claim 21, wherein the port comprises a tapered profile, and wherein an angle of the tapered profile ranges from about 0 degrees to about 45 degrees.

23. (canceled)

24. (canceled)

25. The cartridge assembly of claim 21, wherein a diameter at a distal end of the port is less than a diameter at a proximal end of the port.

26. The cartridge assembly of claim 21, wherein the inlet component comprises one or more turn features that are configured to induce a change in direction of flow of the blood sample for counteracting gravitational force on the flow.

27. (canceled)

28. (canceled)

29. The cartridge assembly of claim 26, wherein the inlet component comprises a reservoir that is configured to collect, aggregate, or pool a volume of the blood sample as wicking of another portion of the blood sample occurs along the matrix.

30. (canceled)

31. (canceled)

32. The cartridge assembly of claim 21, wherein the inlet component comprises a pressure bar that is configured to regulate a flow speed of the blood sample and ensure proper wicking of the blood sample along the matrix for optimal separation of the plasma from the blood sample.

33.-37. (canceled)

38. The cartridge assembly of claim 21, wherein the backing plate comprises one or more spacers that are configured to create a gap between the inlet component and the backing plate.

39. The cartridge assembly of claim 38, wherein the gap is configured to be used in part with a pressure bar on the inlet component to regulate a flow speed of the blood sample and ensure proper wicking of the blood sample along the matrix.

40.-47. (canceled)

48. The cartridge assembly of claim 21, wherein the elongated strip further comprises a substrate on which the matrix is supported.

49. (canceled)

50. (canceled)

51. (canceled)

52. The cartridge assembly of claim 48, wherein the elongated strip further comprises a liner disposed between and separating the substrate and the matrix.

53.-56. (canceled)

57. The cartridge assembly of claim 21, wherein a ratio of a length to a width of the elongated strip is about 2.3:1 to about 7:1.

58.-61. (canceled)

62. The cartridge assembly of claim 21, wherein a distance from a distal end of the port to a proximal end of the elongated strip is about 5 mm to about 15 mm.

63. (canceled)

64. The cartridge assembly of claim 21, wherein a distance from a distal end of the port to a distal end of the elongated strip is about 35 mm to about 115 mm.

65. (canceled)

66. The cartridge assembly of claim 32, wherein a distance from an edge of the reservoir to the pressure bar is about 0 mm to about 5 mm.

67.-73. (canceled)

74. The cartridge assembly of claim 32, wherein a ratio of a width to a length of the pressure bar is about 5:1 to about 14:1.

75.-77. (canceled)

78. The cartridge assembly of claim 32, wherein an edge of the elongated strip is at a distance of about 0 mm to about 10 mm from the pressure bar.

79.-82. (canceled)

83. The cartridge assembly of claim 38, wherein a size of the gap is about 0 mm to about 4 mm.

84. The cartridge assembly of claim 38, wherein the pressure bar comprises the gap.

85.-93. (canceled)

94. The cartridge assembly of claim 21, wherein a ratio of an area of the cartridge assembly to an area of the elongated strip is about 1.5:1 to 2:1.

95. The cartridge assembly of claim 94, wherein the ratio is about 1.8:1.
